(19) United States
(12) Patent Application Publication
(10) Pub. No.: US 2026/0015421 A1
(43) Pub. Date: Jan. 15, 2026

(54) Non-Signaling Chimeric Antigen Receptor Gamma Delta T-Cells

(71) Applicant: IN8bio, Inc., New York, NY (US)

(72) Inventors: Lei DING, Vestavia Hills, AL (US); Yanjie LI, Homewood, AL (US); Lawrence S. LAMB, JR., Birmingham, AL (US)

(21) Appl. No.: 19/295,942

(22) Filed: Aug. 11, 2025

Related U.S. Application Data
(63) Continuation of application No. PCT/US2024/016112, filed on Feb. 16, 2024.
(60) Provisional application No. 63/446,484, filed on Feb. 17, 2023.
(60) Provisional application No. 63/459,910, filed on Apr. 17, 2023.

(51) Int. Cl. C07K 16/28; A61K 40/11; A61K 40/31; A61K 40/35; A61K 40/42; A61P 35/00; C07K 14/54; C07K 14/55; C12N 5/0783

(52) U.S. Cl. CPC C07K 16/2803; A61K 40/11; A61K 40/31; A61K 40/35; A61K 40/421; A61K 40/4211; A61K 40/4224; A61P 35/00; C07K 14/5403; C07K 14/5418; C07K 14/5443; C07K 14/55; C07K 16/2866; C12N 5/0636; C07K 2317/31; C07K 2317/622; C12N 2510/00

(57) ABSTRACT

Described are γδ T-cells that express a non-signaling chimeric antigen receptor (CAR), wherein the CAR binds a tumor antigen such as CD19 or CD33. Also described are pharmaceutical compositions thereof and the method for the treatment of cancer such as leukemia.

### RELATED APPLICATIONS

This application is a continuation of International Application No. PCT/US24/16112, which designated the United States and was filed on Feb. 26, 2024, published in English, which claims the benefit of U.S. Provisional Application No. 63/446,484 filed Feb. 17, 2023, and U.S. Provisional Application No. 63/459,910 filed Apr. 17, 2023. The entire contents of each of the above applications are incorporated herein by reference.

### BACKGROUND OF THE INVENTION

Chimeric antigen receptors (CARs) are composed of an extracellular tumor recognition/targeting domain, an extracellular linker/hinge domain, a transmembrane domain, and intracellular T-cell-activating domain and co-stimulatory signaling domains. The majority of CAR tumor-targeting domains are single chain variable fragments (scFvs) derived from antibody sequences that exploit the specificity of antibody binding to particular antigens. For example, the anti-CD19 targeted CARs KYMRIAH™, and YESCARTA™ are approved therapies for the treatment of acute lymphoblastic leukemia (ALL) and diffuse large B-cell lymphoma, respectively, and comprise scFvs derived from a murine anti-human CD19 antibody (Guedan et al. (2019), Mol Ther Methods Clin Dev 12:145-156). Current CAR-T cell therapies target tumor associated antigens (TAA) expressed on both malignant and cancer cells. αβ cytotoxic T-lymphocytes (CTLs) are narrowly specific for TAA peptides but will bind any cell expressing the target antigen.

Chimeric antigen receptor T cell (CAR-T) therapy for acute lymphocytic leukemia (ALL) and B-cell derived lymphomas have shown remarkable responses in the clinic. However, the current practice of targeting pan-B cell antigens, such as CD19, leads to total aplasia of cells expressing the target CD19 and life-long impairment of humoral immunity. Such on-target off-tumor toxicities create significant challenges in the current COVID-19 environment and in non-B cell malignancies such as acute myeloid leukemia (AML) as well as solid tumor cancers where the target tumor antigen may also be widely expressed on hematopoietic stem cells (HSCs) and/or other healthy tissues in the body. With respect to AML treatment specifically, current CAR T/NK cell therapies target CLL-1, CD33 or CD123 but progress is hindered by the lack of a specific AML tumor associated antigen (TAA). On-target/off-tumor cytotoxicity to hematopoietic stem cells and hematopoietic progenitor cells leads to impaired hematopoiesis. Targeting of TAAs by CAR T-cells is also associated with a particular challenge in the targeting solid tumor cancers because the antigen may be expressed by surrounding healthy tissue in the organ(s) where the tumor resides. This can lead to significant on-target off-tumor effects that results in many highly expressed tumor targets being considered “undruggable” as the potential for significant side-effects could harm the patient.

Gamma-delta (γδ) T-cells are an important subset of T lymphocytes as they can recognize a broad range of antigens without antigen priming or the presence of major histocompatibility complex (MHC) molecules. They can target and kill cells directly through their cytotoxic activity or indirectly through the activation of other immune cell types. γδ T-cell functional responses are induced by several factors including the recognition of stress antigens, which promotes cytokine production and regulates pathogen clearance, inflammation, and tissue homeostasis in response to stress (e.g., a chemotherapeutic agent environment). The cytotoxicity of γδ T-cells to tumors can be induced through the expression of cell surface receptors, including natural killer group 2D ligand (NKG2DL), on tumor cells. Thus, 76 T cells are a unique subset of T lymphocytes that can directly kill malignant cells through the recognition of tumor and/or stress antigens that are not generally expressed on normal healthy tissue including hematopoietic cells.

There is a need to address the limitations of current CAR-T cell therapies and develop better and safer next generation CAR-T therapies.

### SUMMARY OF THE INVENTION

The present invention is based, at least partially, on the unexpected discovery that non-signaling CD19 CAR γδ T cells (γδ T cells transduced with a CAR that targets CD19 and that have a single co-stimulatory domain but lack a T-cell activation domain) are cytotoxic against CD19+ cancer cells while showing minimal cytotoxicity against CD19+ B cells, and the discovery that non-signaling CD33 CAR γδ T cells (γδ T cells transduced with a CAR that targets CD33 and that have a single co-stimulatory domain but lack a T-cell activation domain) are cytotoxic against CD33+ cancer cells while showing minimal cytotoxicity against healthy cells. The invention encompasses γδ T-cells that express a non-signaling CAR (a CAR that lacks a T-cell activation domain, e.g., CD3z, in the endodomain) wherein the CAR binds a tumor antigen such as CD19 or CD33, and optionally binds to a second tumor antigen; gd T-cells that further express a second non-signaling CAR that binds to a different tumor antigen than the first CAR; γδ T-cells that express the non-signaling CAR and that co-express a survival factor; γδ T-cells that express the non-signaling CAR and that co-express a cytokine such as IL-15, IL-2, or IL-7; γδ T-cells that express a non-signaling CAR, wherein the CAR comprises only one co-stimulatory domain; a population of the γδ T-cells that express a non-signaling CAR described herein; pharmaceutical compositions comprising the non-signaling CAR γδ T-cells; and methods of treating cancer or a tumor in a subject in need thereof comprising administering an effective amount of the non-signaling CAR γδ T-cells, and optionally co-administering a chemotherapeutic agent, e.g., the chemotherapeutic agent to which the survival factor confers resistance.

The invention encompasses an engineered γδ T-cell that expresses a chimeric antigen receptor (CAR), wherein the CAR comprises an extracellular antigen-binding domain that binds to a tumor antigen, wherein the CAR does not comprise an intracellular T-cell activation domain and optionally, the CAR comprises a co-stimulatory domain. In certain aspects, the CAR comprises an extracellular hinge domain and/or a transmembrane domain. In some aspects, the non-signaling CAR comprises only one co-stimulatory domain. In certain aspects, the non-signaling CAR comprises one or more co-stimulatory domains. In certain additional embodiments, the CAR does not comprise a co-stimulatory domain. The CAR can, for example, specifically bind only one tumor antigen (referred to herein, as a monoCAR) or can specifically bind two tumor antigens (referred to herein as a tandem dualCAR). The γδ T-cell can, for example, comprise an extracellular antigen-binding domain that binds to CD19 alone (or in other words, a monoCAR that only specifically binds to CD19), or that binds to CD19 and a second tumor antigen (or in other words, a tandem dualCAR that specifically binds to CD19 and a second tumor antigen). In another aspect, the γδ T-cell comprises an extracellular antigen-binding domain that binds CD33 alone (or in other words, a monoCAR that only specifically binds to CD33), or that binds to CD33 and a second tumor antigen, such as CD123 (or in other words, a tandem dualCAR that specifically binds to CD33 and a second tumor antigen, such as CD123). In certain aspects, the CAR is a monoCAR and comprises an anti-CD19 scFv. In a further aspect, the CAR is a monoCAR and comprises an anti-CD33 scFv. In yet additional aspects, the CAR is a tandem dual CAR and binds to CD33 and CD123; for example, the extracellular antigen-binding domain comprises an anti-CD33 scFv and an anti-CD123 scFv, or comprises an anti-CD33 scFv and an IL3 molecule (that specifically binds CD123). In further aspects, the CAR is a tandem dualCAR that binds to CD19 and a second tumor antigen selected from CD20 and CD22; for example the extracellular antigen-binding domain of the CAR comprises anti-CD19 scFv and anti-CD20 scFv or an anti-CD22 scFv. In certain aspects, the gd T-cell expresses a survival factor. In yet additional aspects, the gd T-cell further expresses a cytokine selected from the group consisting of interleukin-15 (IL15), interleukin-2 (IL2), and interleukin-7 (IL7). In certain aspects, the gd T-cell further expresses IL15.

The invention includes an engineered γδ T-cell that expresses a chimeric antigen receptor (CAR), wherein the CAR comprises:
i. an extracellular antigen-binding domain that binds to a tumor antigen; a non-limiting example of a tumor antigen is CD19 or CD33;
ii. a transmembrane domain;
iii. an optional extracellular hinge domain; and
iv. an optional co-stimulatory domain;
   wherein the CAR does not comprise an intracellular T-cell activation domain. An engineered γδ T-cell that expresses a CAR that does not comprise or include an intracellular T-cell activation domain such as CD3z signaling domain is referred to herein as a “non-signaling CAR γδ T-cell.” In certain aspects, the non-signaling CAR comprises one or more co-stimulatory domains. In certain aspect, the extracellular hinge domain is present. In some aspects, the non-signaling CAR comprises only one co-stimulatory domain. In certain embodiments, the CAR does not comprise a co-stimulatory domain. The CAR can, for example, specifically bind only one tumor antigen (referred to herein, as a monoCAR) or can specifically bind two tumor antigens (referred to herein as a tandem dualCAR). The γδ T-cell can, for example, comprise an extracellular antigen-binding domain that binds to CD19 alone (or in other words, a monoCAR that only specifically binds to CD19), or that binds to CD19 and a second tumor antigen (or in other words, a tandem dualCAR that specifically binds to CD19 and a second tumor antigen). In another aspect, the γδ T-cell comprises an extracellular antigen-binding domain that binds CD33 alone (or in other words, a monoCAR that only specifically binds to CD33), or that binds to CD33 and a second tumor antigen, such as CD123 (or in other words, a tandem dualCAR that specifically binds to CD33 and a second tumor antigen, such as CD123). In certain aspects, the CAR is a monoCAR and comprises an anti-CD19 scFv. In a further aspect, the CAR is a monoCAR and comprises an anti-CD33 scFv. In yet additional aspects, the CAR is a tandem dual CAR and binds to CD33 and CD123; for example, the extracellular antigen-binding domain comprises an anti-CD33 scFv and an anti-CD123 scFv, or comprises an anti-CD33 scFv and an IL3 molecule (that specifically binds CD123). In further aspects, the CAR is a tandem dualCAR that binds to CD19 and a second tumor antigen selected from CD20 and CD22; for example the extracellular antigen-binding domain of the CAR comprises anti-CD19 scFv and anti-CD20 scFv or an anti-CD22 scFv. In certain aspects, the gd T-cell expresses a survival factor.

In some embodiments, the invention is an engineered γδ T-cell that expresses a chimeric antigen receptor (CAR), wherein the γδ T-cell further expresses a survival factor, wherein the survival factor is a DNA, RNA, or polypeptide that confers resistance to a chemotherapeutic agent, and further wherein the CAR comprises:
i. an extracellular antigen-binding domain that binds to a tumor antigen;
ii. a transmembrane domain;
iii. an extracellular hinge domain; and
iv. an optional co-stimulatory domain;
   wherein the CAR does not comprise an intracellular T-cell activation domain. In certain aspects the tumor antigen is CD19 or CD33. In additional aspects, the CAR comprises one or more co-stimulatory domains. In some aspects, the CAR comprises only one co-stimulatory domain. In certain embodiments, the CAR does not comprise a co-stimulatory domain. In yet additional aspects, the γδ T-cell further expresses a cytokine selected from the group consisting of interleukin-15 (IL15), interleukin-2 (IL2), and interleukin-7 (IL7). The cytokine can be membrane-tethered or secreted. The CAR can, for example, be a monoCAR or a tandem dualCAR. The γδ T-cell can, for example, comprise an extracellular antigen-binding domain that binds to CD19 alone (a monoCAR that only specifically binds to CD19), or that binds to CD19 and a second tumor antigen (a tandem dualCAR that specifically binds to CD19 and a second TAA). In another aspect, the γδ T-cell can comprise an extracellular antigen-binding domain that binds CD33 alone (a monoCAR that only specifically binds to CD33), or that binds to CD33 and a second tumor antigen, such as CD123 (a tandem dualCAR that can specifically bind to CD33 and a second tumor antigen, such as CD123). In certain aspects, the CAR is a monoCAR and comprises an anti-CD19 scFv. In a further aspect, the CAR is a monoCAR and comprises an anti-CD33 scFv. In yet additional aspects, the CAR is a tandem dual CAR and binds to CD33 and CD123; for example, the extracellular antigen-binding domain comprises an anti-CD33 scFv and an anti-CD123 scFv, or comprises an anti-CD33 scFv and an IL3 molecule, for example, human IL3. In further aspects, the CAR is a tandem dualCAR that binds to CD19 and a second tumor antigen selected from CD20 and CD22; for example the extracellular antigen-binding domain comprises anti-CD19 scFv and an anti-CD20 scFv or an anti-CD22 scFv.

In additional aspects, the invention is an engineered γδ T-cell that expresses a chimeric antigen receptor (CAR), wherein the CAR comprises:
i. an extracellular antigen-binding domain that binds to a tumor antigen;
ii. a transmembrane domain;
iii. an extracellular hinge domain; and
iv. an optional co-stimulatory domain;
   wherein the CAR does not comprise an intracellular T-cell activation domain and wherein the γδ cell further expresses a cytokine selected from the group consisting of interleukin-15 (IL15), interleukin-2 (IL2), and interleukin-7 (IL7). In certain aspects, the cytokine is IL15. The cytokine can be membrane-tethered or secreted. In certain aspects, the tumor antigen is CD19 or CD33. In yet additional aspects, the γδ T-cell is further engineered to express a survival factor. In certain embodiments, the CAR does not comprise a co-stimulatory domain. In additional aspects, the CAR comprises one or more co-stimulatory domains. In some aspects, the CAR comprises only one co-stimulatory domain. The CAR can for example, be a monoCAR or a tandem dualCAR.

The γδ T-cell can, for example, comprise an extracellular antigen-binding domain that binds to CD19 alone (a monoCAR that only specifically bind to CD19), or that binds to CD19 and a second tumor antigen (a tandem dualCAR that specifically binds to CD19 and a second TAA). In another aspect, the γδ T-cell can comprise an extracellular antigen-binding domain that binds CD33 alone (a monoCAR that can specifically bind to CD33), or that binds to CD33 and a second tumor antigen, such as CD123 (a tandem dualCAR that specifically binds to CD33 and a second tumor antigen, such as CD123). In certain aspects, the CAR is a monoCAR and comprises an anti-CD19 scFv. In a further aspect, the CAR is a monoCAR and comprises an anti-CD33 scFv. In yet additional aspects, the CAR is a tandem dualCAR and binds to CD33 and CD123; for example, the extracellular antigen-binding domain comprises an anti-CD33 scFv and an anti-CD123 scFv, or an anti-CD33 scFv and IL3 molecule, for example, human IL3. In further aspects, the CAR is a tandem dualCAR that binds to CD19 and a second tumor antigen selected from CD20 and CD22; for example, the extracellular antigen-binding domain comprises anti-CD19 scFv and anti-CD20 scFv or an anti-CD22 scFv.

In yet additional aspects, the invention is an engineered γδ T-cell that expresses a chimeric antigen receptor (CAR), wherein the CAR comprises:
i. an extracellular antigen-binding domain that binds to a tumor antigen;
ii. a transmembrane domain;
iii. an extracellular hinge domain that attaches the transmembrane domain to the extracellular antigen-binding domain; and
iv. only one co-stimulatory domain;
wherein the CAR does not comprise an intracellular T-cell activation domain. In certain aspects the tumor antigen is CD19 or CD33. In yet additional aspects, the γδ T-cell is engineered to further express a survival factor. In further aspects, the γδ T-cell further expresses a cytokine selected from the group consisting of interleukin-15 (IL15), interleukin-2 (IL2), and interleukin-7 (IL7). In certain aspects, the cytokine is IL15. The cytokine can be membrane-tethered or secreted. The CAR can, for example, be a monoCAR or a tandem dualCAR. The γδ T-cell can, for example, comprise an extracellular antigen-binding domain that binds to CD19 alone (a monoCAR that can only specifically bind to CD19), or that binds to CD19 and a second tumor antigen (a tandem dualCAR that can specifically bind to CD19 and a second TAA). In another aspect, the γδ T-cell can comprise an extracellular antigen-binding domain that binds CD33 alone (a monoCAR that can specifically bind to CD33), or that binds to CD33 and a second tumor antigen, such as CD123 (a tandem dualCAR that specifically binds to CD33 and a second tumor antigen, such as CD123). In certain aspects, the CAR is a monoCAR and comprises an anti-CD19 scFv. In a further aspect, the CAR is a monoCAR and comprises an anti-CD33 scFv. In yet additional aspects, the CAR is a tandem dual CAR and binds to CD33 and CD123; for example, the extracellular antigen-binding domain comprises an anti-CD33 scFv and an anti-CD123 scFv or an anti-CD33 scFv and an IL3 molecule, for example, human IL3. In further aspects, the CAR is a tandem dualCAR that binds to CD19 and a second tumor antigen selected from CD20 and CD22; for example the extracellular antigen-binding domain comprises anti-CD19 scFv and anti-CD20 scFv or an anti-CD22 scFv.

In further embodiments, the invention is an engineered γδ T-cell that expresses a CAR, wherein the CAR comprises:
i. an extracellular antigen-binding domain that binds to a tumor antigen;
ii. a transmembrane domain;
iii. an extracellular hinge domain that attaches the transmembrane domain to the extracellular antigen-binding domain;
iv. only one co-stimulatory domain; and
wherein the CAR does not comprise an intracellular T-cell activation domain and wherein the γδ T cell further expresses a cytokine selected from the group consisting of interleukin-15 (IL15), interleukin-2 (IL2), and interleukin-7 (IL7). In certain aspects, the co-expressed cytokine is IL15. The cytokine can be membrane-tethered or secreted. In certain aspects, the CAR is a monoCAR or a tandem dual CAR comprises an anti-CD19 antibody or fragment thereof, such as an scFv. In a further aspect, the CAR is a monoCAR and comprises an anti-CD19 scFv. In certain additional aspects, the CAR is a monoCAR or a tandem dual CAR comprises an anti-CD33 antibody or fragment thereof, such as an scFv. In a further aspect, the CAR is a monoCAR or a tandem dual CAR comprises an anti-CD33 antibody or fragment thereof, such as an scFv. In certain aspects, the CAR is a monoCAR and comprises an anti-CD33 scFv. In other aspects, the CAR is a tandem dualCAR comprising an anti-CD33 scFv and anti CD123 scFv or an IL3 molecule, for example, human IL3. In certain aspects, the γδ T-cell further expresses a survival factor, wherein the survival factor is a DNA, RNA, or polypeptide that confers resistance to a chemotherapeutic agent.

In further embodiments, the invention is an engineered γδ T-cell that expresses a monoCAR, wherein the CAR comprises:
i. an extracellular antigen-binding domain that binds to CD19;
ii. a transmembrane domain;
iii. an extracellular hinge domain that attaches the transmembrane domain to the extracellular antigen-binding domain;
iv. only one co-stimulatory domain; and
wherein the CAR does not comprise an intracellular T-cell activation domain and wherein the γδ T cell further expresses a cytokine selected from the group consisting of interleukin-15 (IL15), interleukin-2 (IL2), and interleukin-7 (IL7). In certain aspects, the co-expressed cytokine is IL15. The cytokine can be membrane-tethered or secreted. In certain aspects, the CAR is a monoCAR or dual CAR comprises an anti-CD19 antibody or fragment thereof, such as an scFv. In a further aspect, the CAR is a monoCAR and comprises an anti-CD19 scFv. In certain aspects, the γδ T-cell further expresses a survival factor, wherein the survival factor is a DNA, RNA, or polypeptide that confers resistance to a chemotherapeutic agent.

In yet additional embodiments, the invention is an engineered γδ T-cell that expresses a monoCAR, wherein the CAR comprises:
i. an extracellular antigen-binding domain that binds to CD33;
ii. a transmembrane domain;
iii. an extracellular hinge domain that attaches the transmembrane domain to the extracellular antigen-binding domain;
iv. only one co-stimulatory domain; and
wherein the CAR does not comprise an intracellular T-cell activation domain and wherein the γδ T cell further expresses a cytokine selected from the group consisting of interleukin-15 (IL15), interleukin-2 (IL2), and interleukin-7 (IL7). In certain aspects, the co-expressed cytokine is IL15. The cytokine can be membrane-tethered or secreted. In certain aspects, the CAR is a monoCAR or dual CAR comprises an anti-CD33 antibody or fragment thereof, such as scFv. In a further aspect, the CAR is a monoCAR and comprises an anti-CD33 scFv. In certain aspects, the γδ T-cell further expresses a survival factor, wherein the survival factor is a DNA, RNA, or polypeptide that confers resistance to a chemotherapeutic agent.

In further embodiments, the invention is an engineered γδ T-cell that expresses a tandem dualCAR, wherein the CAR comprises:
i. an extracellular antigen-binding domain that binds to CD19 and a second tumor antigen; optionally, the second tumor antigen is CD20 or CD22;
ii. a transmembrane domain;
iii. an extracellular hinge domain that attaches the transmembrane domain to the extracellular antigen-binding domain;
iv. only one co-stimulatory domain; and
wherein the CAR does not comprise an intracellular T-cell activation domain and
wherein the γδ T cell further expresses a cytokine selected from the group consisting of interleukin-15 (IL15), interleukin-2 (IL2), and interleukin-7 (IL7). In certain aspects, the co-expressed cytokine is IL15. The cytokine can be membrane-tethered or secreted. In certain aspects, the tandem dual CAR comprises an anti-CD19 antibody or fragment thereof, such as scFv. In further aspects, the tandem dualCAR comprises an anti-CD19 antibody or fragment thereof and an comprises an anti-CD20 antibody or fragment thereof. In yet further aspects, the tandem dual CAR comprises an anti-CD19 antibody or fragment thereof and an comprises an anti-CD22 antibody or fragment thereof. In certain aspects, the γδ T-cell further expresses a survival factor, wherein the survival factor is a DNA, RNA, or polypeptide that confers resistance to a chemotherapeutic agent.

In further aspects, the invention is an engineered γδ T-cell that expresses a tandem dual CAR, wherein the CAR comprises:
i. an extracellular antigen-binding domain that binds to CD33 and a second tumor antigen such as CD123;
ii. a transmembrane domain;
iii. an extracellular hinge domain that attaches the transmembrane domain to the extracellular antigen-binding domain;
iv. only one co-stimulatory domain; and
wherein the dualCAR does not comprise an intracellular T-cell activation domain and wherein the γδ T cell further expresses a cytokine selected from the group consisting of interleukin-15 (IL15), interleukin-2 (IL2), and interleukin-7 (IL7). In certain aspects, the co-expressed cytokine is IL15. The cytokine can be membrane-tethered or secreted. In additional aspects, the second tumor antigen is CD123. In certain aspects, the CAR comprises an anti-CD33 antibody or fragment thereof, such as scFv, and comprises an anti-CD123 antibody of fragment thereof, such as an scFv. In another embodiment, the CAR comprises an anti-CD33 antibody or fragment thereof, such as scFv, and comprises an IL3 molecule, such as a human IL3. In certain aspects, the γδ T-cell further expresses a survival factor, wherein the survival factor is a DNA, RNA, or polypeptide that confers resistance to a chemotherapeutic agent.

The invention additionally includes an engineered γδ T-cell that expresses a first CAR and a second CAR,
wherein the first CAR comprises:
i. an extracellular antigen-binding domain that binds to a first tumor antigen; a non-limiting example of a first tumor antigen is CD19 or CD33;
ii. a transmembrane domain;
iii. an extracellular hinge domain; and
iv. an optional co-stimulatory domain;
wherein the first CAR does not comprise an intracellular T-cell activation domain; and
wherein the second CAR comprises:
i. an extracellular antigen-binding domain that binds to a second tumor antigen;
wherein the second tumor antigen is different from the first tumor antigen
ii. a transmembrane domain;
iii. an extracellular hinge domain; and
iv. an optional co-stimulatory domain;
   wherein the second CAR does not comprise an intracellular T-cell activation domain. An engineered γδ T-cell that expresses a first CAR that does not comprise or include an intracellular T-cell activation domain such as CD3z signaling domain and that expresses a second CAR that does not comprise an intracellular T-cell activation domain is referred to herein as a “non-signaling separate dualCAR.” In certain aspects, each of the first and/or second CAR of the non-signaling separate dualCAR comprises one or more co-stimulatory domains. In some aspects, each of the first CAR and the second CAR comprises only one co-stimulatory domain. In certain aspects, the first tumor antigen is CD19; optionally the antigen-binding domain of the first CAR comprises an anti-CD19 scFv. In yet additional aspects, the first tumor antigen is CD19 and the second tumor antigen is CD20 or CD22; optionally the antigen-binding domain of the first CAR comprises an anti-CD19 scFv and the antigen-binding domain of the second CAR comprises an anti-CD20 scFv or anti-CD22 scFv. In other aspects, the first tumor antigen is CD33; optionally the antigen-binding domain of the first CAR comprises an anti-CD33 scFv. In further aspects, the first tumor antigen is CD33 and the second tumor antigen is CD123; optionally the antigen-binding domain of the first CAR comprises an anti-CD33 scFv and the antigen-binding domain of the second CAR comprises an anti-CD123 scFv or an IL3 molecule. In certain aspects, the gd T-cell expresses a survival factor. In further aspects, the gd T-cell further expresses a cytokine selected from the group consisting of interleukin-15 (IL15), interleukin-2 (IL2), and interleukin-7 (IL7). In certain aspects, the co-expressed cytokine is IL15. The cytokine can be membrane-tethered or secreted. In yet further aspects, the gd T-cell expresses a survival factor and further expresses a cytokine selected from the group consisting of interleukin-15 (IL15), interleukin-2 (IL2), and interleukin-7 (IL7). In additional embodiments, each of the first CAR and the second CAR comprises only one co-stimulatory domain and the gd T-cell further expresses a cytokine selected from the group consisting of interleukin-15 (IL15), interleukin-2 (IL2), and interleukin-7 (IL7). In yet a further embodiment, each of the first CAR and the second CAR comprises only one co-stimulatory domain and the gd T-cell expresses a survival factor. In another embodiment, each of the first CAR and the second CAR comprises only one co-stimulatory domain, the gd T-cell expresses a survival factor, and the gd T-cell further expresses a cytokine selected from the group consisting of interleukin-15 (IL15), interleukin-2 (IL2), and interleukin-7 (IL7).

The invention additionally includes non-signaling CARs as described herein and above that do not include an extracellular hinge domain.

The invention also encompasses a population of the engineered γδ T-cells described herein.

The invention additionally includes a pharmaceutical composition comprising the non-signaling CAR γδ T-cells described herein, as well as a method of treating cancer or tumor in a subject in need thereof, the method comprising administering to said subject a composition comprising the non-signaling CAR γδ T-cells as described herein. In some embodiments, the pharmaceutical composition further comprises an NK cell or γδ T cells, or a combination thereof. The γδ T cells can be present at greater than or equal to 60% of the total cell population as determined by flow cytometry and optionally, the γδ T cells are present at less than or equal to 5% of the total cell population as determined by flow cytometry. The methods of treating cancer or tumor can optionally comprise co-administering to said subject an effective amount of the chemotherapeutic agent; for example, the effective amount is an amount sufficient to increase stress antigen expression on the cancer or tumor cells. In certain aspects, the cancer is ALL and the non-signaling CAR γδ T-cells are monoCAR γδ T cells that binds to CD19, a tandem dualCAR γδ T cells that binds to CD19 and a second tumor antigen, or a separate dualCAR wherein the first CAR binds to CD19 and the second CAR binds to a second tumor antigen. In yet additional aspects, the cancer is AML and the non-signaling CAR γδ T-cell is a monoCAR γδ T cell that binds to CD33, a tandem dualCAR γδ T cell that binds to CD33 and a second tumor antigen, or a separate dualCAR wherein the first CAR binds to CD33 and the second CAR binds to a second tumor antigen; for example, the second tumor antigen is CD123.

In certain aspects, the survival factor is a polypeptide that confers resistance to a chemotherapeutic agent. The polypeptide that confers resistance to a chemotherapeutic agent can, for example, be selected from the group consisting of alkyl guanine transferase (AGT), O⁶ methylguanine DNA methyltransferase (MGMT), P140K MGMT (also referred to herein as MGMTp140k), L22Y-DHFR, thymidylate synthase, dihydrofolate reductase, multidrug resistance protein 1 (MDR1), 5′ nucleotidase II, dihydrofolate reductase, and thymidylate synthase. The polypeptide can, for example, confer resistance to any chemotherapeutic agent, for example an alkylating agent. In additional aspects, the polypeptide confers resistance to a chemotherapeutic agent selected from the group consisting of trimethotrexate, temozolomide, raltitrexed, S-(4-Nitrobenzyl)-6-thioinosine, 6-benzyguanidine, nitrosoureas, fotemustine, cytarabine, camptothecin, vincristine, daunorubicin, doxorubicin, L-asparaginase, PEG-L-asparaginase, 6-mercaptopurine, methotrexate, trimetrexate (TMTX), cyclophosphamide, chlorambucil, bendamustine, ifosfamide, cisplatin, carboplatin, oxaliplatin, fludarabine, pentostatin, cladribine, cytarabine, gemcitabine, methotrexate, pralatrexate, mitoxantrone, etoposide and bleomycin. In additional aspects, the chemotherapeutic agent is one or more agents used in the treatment of ALL including, for example, vincristine, doxorubicin, cyclophosphamide, cytarabine, asparaginase and methotrexate. In further aspects, the chemotherapeutic agent is one or more chemotherapeutic agents used in the treatment of AML including, for example, cytarabine, daunorubicin, idarubicin, mitoxantrone, etoposide, thioguanine and fludarabine.

In yet additional aspects, the survival factor is a DNA or an RNA.

In certain aspects, the CAR gd T cells expresses more than one survival factor. For example, the CAR gd T cells can express two survival factors.

The non-signaling CAR γδ T cells described herein can additionally express a suicide gene. A non-limiting example of a suicide gene is thymidine kinase, for example, the herpes simplex virus thymidine kinase (HSV-TK).

The CAR can comprise a transmembrane domain, for example, comprising a CD28 transmembrane domain; and/or a hinge domain, for example, that comprises the hinge region of a protein selected from the group consisting of CD8, CD28, and/or CD137. In certain aspects, the co-stimulatory domain is present and comprises the CD28 co-stimulatory domain, the OX40 co-stimulatory domain, and/or the 4-1BB co-stimulatory domain. In additional aspects, only one co-stimulatory domain is present. The CAR can comprise one or more linker peptides.

Exemplary linker peptides are c-myc, FLAG, and (GSSS)ₙ. The CAR can additionally comprise an extracellular signal peptide; for example, the signal peptide is the signal peptide of a protein selected from the group consisting of CD8, CD28, GM-CSF, CD4, CD137, or a combination thereof. The CAR can comprise a linker peptide; exemplary linker peptides are c-myc, FLAG, HA, and (GSSS)ₙ.

The non-signaling CARs described herein does not comprise or include an intracellular T-cell activation domain, such as the CD3 zeta signaling domain (also referred to herein interchangeably as “CD3z” and “CD3C”. In additional embodiments, the CAR comprises a co-stimulatory domain (e.g., CD28 co-stimulatory domain), including embodiments wherein the CAR comprises only one co-stimulatory domain.

The methods of treatment can, for example, be for the treatment of lymphoma or leukemia.

In certain aspects, the method of treatment is a method of treating acute lymphoblastic leukemia (ALL) comprising administering to a patient in need thereof of an engineered γδ T-cell that expresses a non-signaling chimeric antigen receptor (CAR) that binds CD19 as described herein. In certain aspects, the non-signaling CD19 CAR γδ T cell is a monoCAR. In additional aspects, the non-signaling CD19 CAR γδ T cell is a tandem dual CAR γδ T cell. In other aspects, the gd T cells express a non-signaling separate dual CAR wherein the first CAR binds CD19.

In yet additional aspects, the method is a method of treating acute myeloid leukemia (AML) comprising administering to a patient in need thereof of an engineered γδ T-cell that expresses a non-signaling chimeric antigen receptor (CAR) that binds CD33 as described herein. In certain aspects, the non-signaling CD33 CAR γδ T cell is a monoCAR γδ T cell. In additional aspects, the non-signaling CD33 CAR is a dualCAR γδ T cell. In certain specific aspects, the non-signaling CD33 CAR is a tandem dualCAR γδ T cell that binds to CD33 and CD123. In other aspects, the gd T cells express a non-signaling separate dualCAR wherein the first CAR binds CD33 and the second CAR binds to a second tumor antigen such as CD123.

The stress antigen expressed by the cancer cells and/or upregulated in response to the chemotherapeutic agent can be an NKG2D ligand (NKG2DL). Non-limiting examples of NKG2DLs included, but are not limited to, MIC-A, MIC-B, ULBP-1, ULBP-2, ULBP-3 and ULBP-4.

The non-signaling CAR γδ T-cells described herein (including monoCAR γδ T cells, tandem dualCAR γδ T cells and separate dualCAR gd T cells) can have enhanced cytotoxicity to the cancer or tumor cells as compared to that of γδ T-cells lacking the CAR but that otherwise are identical to the γδ T-cell that lacks the CAR (referred to herein as a “comparable γδ T-cell”). The non-signaling CAR γδ T-cell described herein can have reduced cytotoxicity to normal (non-cancerous) cells that express the tumor antigen as compared to that of a signaling CAR γδ T-cell that is identical to the non-signaling CAR γδ T-cell to which it is compared to except that it comprises an intracellular T-cell activation domain, such as that of CD3z (“comparable signaling CAR γδ T-cell”). In some embodiments, the cytotoxicity of the non-signaling CAR γδ T-cells to normal (non-cancerous) cells is at least about 10%, 20%, 30%, 40%, 50%, 60%, 70%, 80% or 90% less than that of the comparable signaling CAR γδ T-cell (e.g. as measured by percentage of cells killed). For example, the non-signaling CD19 CAR γδ T-cell described herein (including monoCAR γδ T cells and dualCAR γδ T cells) can have reduced cytotoxicity to normal (non-cancerous) B cells that express the tumor antigen as compared to that of a comparable signaling CAR γδ T-cell. The non-signaling CAR αβ T-cell described herein can also have reduced cytotoxicity to normal (non-cancerous) cells that express the tumor antigen as compared to that of a signaling CAR γδ T-cell that has a CAR that is identical to the CAR of the non-signaling CAR γδ T-cell to which it is compared, except that the CAR of the αβ T-cell comprises an intracellular T-cell activation domain, such as that of CD3z, (referred to herein as a “comparable signaling CAR αβ T-cell”). In some embodiments, the cytotoxicity of the non-signaling CAR γδ T-cells to normal (non-cancerous) cells is at least about 10%, 20%, 30%, 40%, 50%, 60%, 70%, 80% or 90% less than that of the comparable signaling CAR αβT-cell (e.g. as measured by percentage of cells killed). For example, the non-signaling CD19 CAR αβT-cell described herein can have reduced cytotoxicity to normal (non-cancerous) B cells that express the tumor antigen as compared to that of a comparable signaling CAR αβ T-cell. In some embodiments, the cytotoxicity of the non-signaling CD19 CAR γδ T-cells to normal B cells is at least about 10%, 20%, 30%, 40%, 50%, 60%, 70%, 80% or 90% less than that of a comparable signaling CD19 CAR αβT-cell. In some embodiments, the cytotoxicity of the non-signaling CD19 CAR γδ T-cells to normal B cells is at least about 10%, 20%, 30%, 40%, 50%, 60%, 70%, 80% or 90% less than that of a comparable signaling CD19 CAR αβT-cell.

In another example, the non-signaling CD33 CAR γδ T-cell described herein (including monoCAR γδ T cells, tandem dualCAR γδ T cells, and separate dualCAR γδ T cells) can have reduced cytotoxicity to normal (non-cancerous) cells, such as early multilineage hematopoietic progenitors, myelomonocytic precursors and normal myeloid cells, that express the tumor antigen (CD33) as compared to that of a comparable signaling CD33 CAR γδ T-cell. In some embodiments, the cytotoxicity of the non-signaling CD33 CAR γδ T-cells to normal non-cancerous cells, such as normal myeloid cells, is at least about 10%, 20%, 30%, 40%, 50%, 60%, 70%, 80% or 90% less than that of a comparable signaling CD33 CAR γδ T-cell. In yet further examples, the non-signaling CD33 CAR γδ T-cell described herein can have reduced cytotoxicity to normal (non-cancerous) cells, such as early multilineage hematopoietic progenitors, myelomonocytic precursors and normal myeloid cells, that express the tumor antigen (CD33) as compared to that of a comparable signaling CD33 CAR γδ T-cell. In some embodiments, the cytotoxicity of the non-signaling CD33 CAR γδ T-cells to normal non-cancerous cells, such as normal myeloid cells, is at least about 10%, 20%, 30%, 40%, 50%, 60%, 70%, 80% or 90% less than less than that of a comparable signaling CD33 CAR αβ T-cell.

In further aspects, the non-signaling CAR γδ T-cell can have enhanced persistence as compared to that of a comparable signaling CAR γδ T-cell. In yet additional aspects, the non-signaling CAR γδ T-cell that co-expresses IL15, IL7 and/or IL2, has enhanced persistence as compared to that of comparable signaling CAR γδ T-cell and/or enhanced persistence as compared to a signaling CAR γδ T-cell that does not co-express the IL15, IL2, and/or IL7. In certain specific aspects, the non-signaling CAR γδ T-cell that co-expresses IL15 has enhanced persistence as compared to that of comparable signaling CAR γδ T-cell and/or enhanced persistence as compared to a signaling CAR γδ T-cell that does not co-express the IL15.

A monoCAR is a CAR that specifically binds only one tumor antigen (referred to herein, as a monoCAR), for example, the extracellular antigen domain has specificity for only one tumor antigen. A monoCAR γδ T cell is a γδ T cell that is engineered to express a monoCAR.

A tandem dualCAR is a CAR (a single CAR) that can specifically bind two tumor antigens, for example, the extracellular antigen binding domain of the CAR has specificity for two tumor antigens. A tandem dualCAR γδ T cell is a γδ T cell that is engineered to express a tandem dualCAR. In tandem expression, both antigen specificities (e.g., two different scFvs) are engineered into a single construct and expressed as a single protein, for example, connected by a flexible linker, on the surface of the γδ T cell. The two antigen specificities are positioned in tandem in the extracellular antigen binding domain.

A γδ T cell that expresses a separate dualCAR expresses a first CAR and a second CAR, wherein each CAR has different antigen specificities. In separate expression, the two different CARs are engineered separately into the γδ cells, each targeting a different specific antigen. For example, the two CARs can be expressed by one viral vector connected by a linker sequence (e.g., a 2A sequence) or can be expressed by two separate vectors.

The term “dualCAR” and “dual CAR” are used interchangeably herein.

As discussed above a “comparable signaling CAR γδ T-cell” is identical to the non-signaling CAR γδ T-cell to which it is compared to except that it comprises an intracellular T-cell activation domain, such as that of CD3z, in its CAR(s). The comparable signaling CAR γδ T-cell is, for example, identical to the non-signaling CAR γδ T-cell to which it is compared to except that it comprises the CD3z intracellular T-cell activation domain. A composition comprising the comparable signaling CAR γδ T-cell is identical to that of the non-signaling CAR γδ T-cell to which it is being compared (e.g., the number of cells is the same; the excipients are the same, the mode of administration is the same, etc.).

Also, a “comparable signaling CAR αβ T-cell” has a CAR that is identical to that of non-signaling CAR γδ T-cell to which it is compared to except that the CAR of the αβ T-cell comprises an intracellular T-cell activation domain, such as that of CD3z. The CAR of the comparable signaling CAR αβ T-cell is, for example, identical to the CAR of the non-signaling CAR γδ T-cell to which it is compared to except that it comprises the CD3z intracellular T-cell activation domain. A composition comprising the comparable signaling CAR αβ T-cell is identical to that of the non-signaling CAR γδ T-cell to which it is being compared (e.g., the number of cells is the same; the excipients are the same, the mode of administration is the same, etc.). The invention further includes methods of enhancing the persistence of CLTX-CAR γδ T-cells in a subject undergoing treatment with a chemotherapeutic agent, the method comprising engineering the γδ T-cells to express the non-signaling CAR as described herein, wherein the non-signaling CAR γδ T-cells (or composition thereof) have enhanced persistence as compared to comparable signaling CAR γδ T-cells (or composition thereof), and further comprising administering the engineered γδ T-cells to the subject. In certain aspects, the non-signaling CLTX-CAR γδ T-cells co-express IL15, IL2 and/or IL7.

The invention additionally includes a nucleic acid or vector encoding the non-signaling CAR as described herein. In certain aspects, the nucleic acid or vector further encodes a survival factor. In yet further aspects, the nucleic acid or vector further encodes IL15, IL2 and/or IL7.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a schematic showing a γδ T-cell that expresses a non-signaling (NS)-CD19 CAR and its interaction with a CD19+ normal cell versus a CD19+ cancer cell. When the NS-CD19 CAR γδ T cell encounters the CD19+ normal (non-cancerous) cell, the anti-CD19 CAR binds to the CD19 on the surface of the normal cell. However, because the NS-CD19 CAR does not include a T-cell activating domain such as CD3z, the binding of the CAR to CD19 does not activate the T cell to kill, and the normal cell is spared. In contrast, when the NS-CD19 CAR γδ T cells encounters the CD19+ cancer cell, the anti-CD19 CAR binds to the CD19 on the surface of the cancer cell and the NKG2D receptor on the γδ T cell binds the stress-induced NKG2D ligand on the cancer cell. The γδ T cell is cytotoxic to the cancer cell because of this interaction between the NKG2D receptor on the γδ T cell and the NKG2D ligand on the cancer. The ability of the NS-CD19 CAR γδ T-cells to discriminate between normal and stressed tissue allows the NS-CD19 CAR γδ T-cells to specifically kill CD19+ cancer cells while sparing normal CD19+ cells, such as normal B cells. Non-signaling CD19 CARs may enhance the efficacy and avidity of the modified γδ T cells to the malignant CD19 cells. With CD19 NSCAR modified γδ T-cells, no activation signal would be transduced from the CAR which may mitigate on-target, off-tumor cytotoxicity to normal B cells. In addition, compared to signaling CARs, non-signaling CARs may mitigate activation-induced cell death and tonic signaling issues, as well as promoting the survival of the modified γδ T cells. Non-signaling CD19 CARs may also prevent killing of CAR γδ-T cells that incorporated CD19 stripped from tumor cells by trogocytosis, enhancing persistence. The CAR T-cell platform described herein spares healthy tissue while targeting cancer cells.

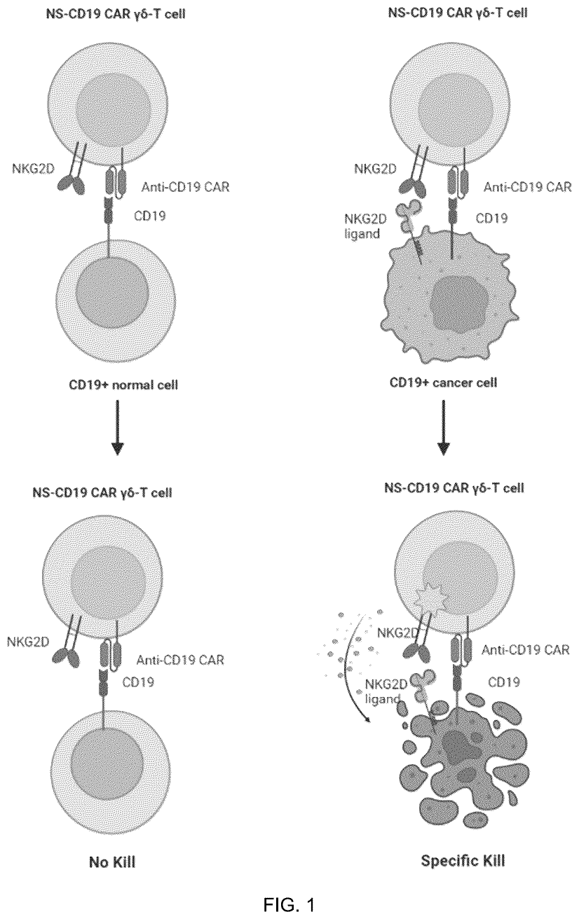

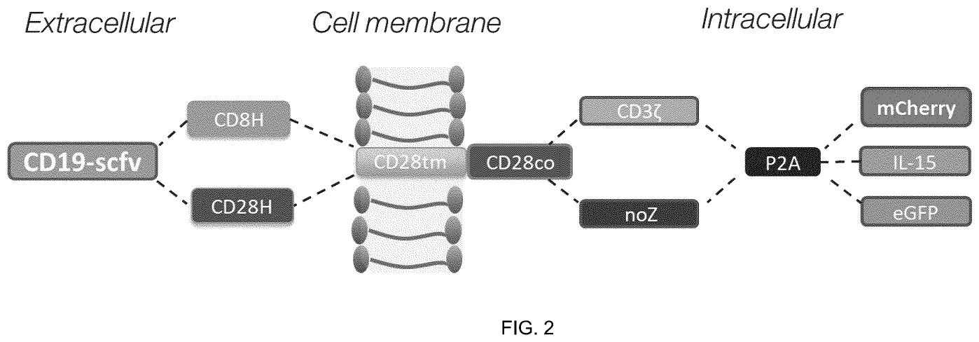

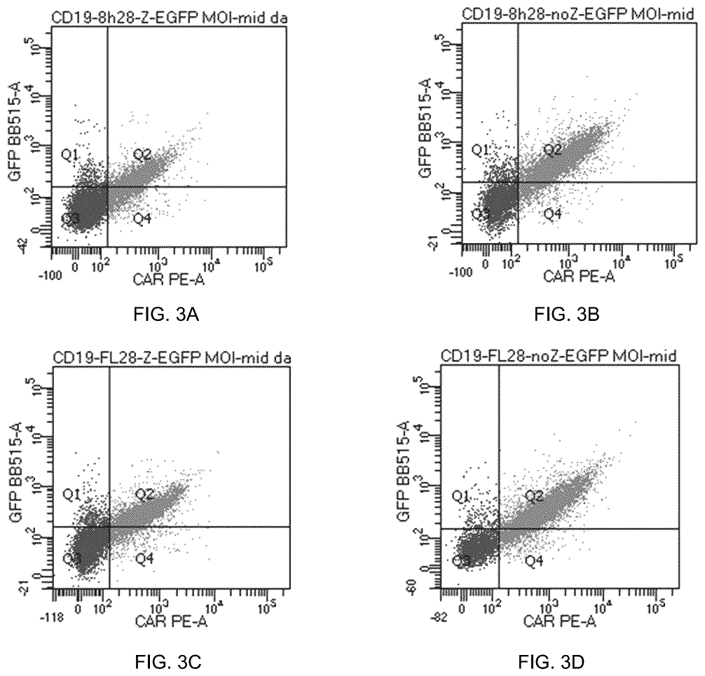

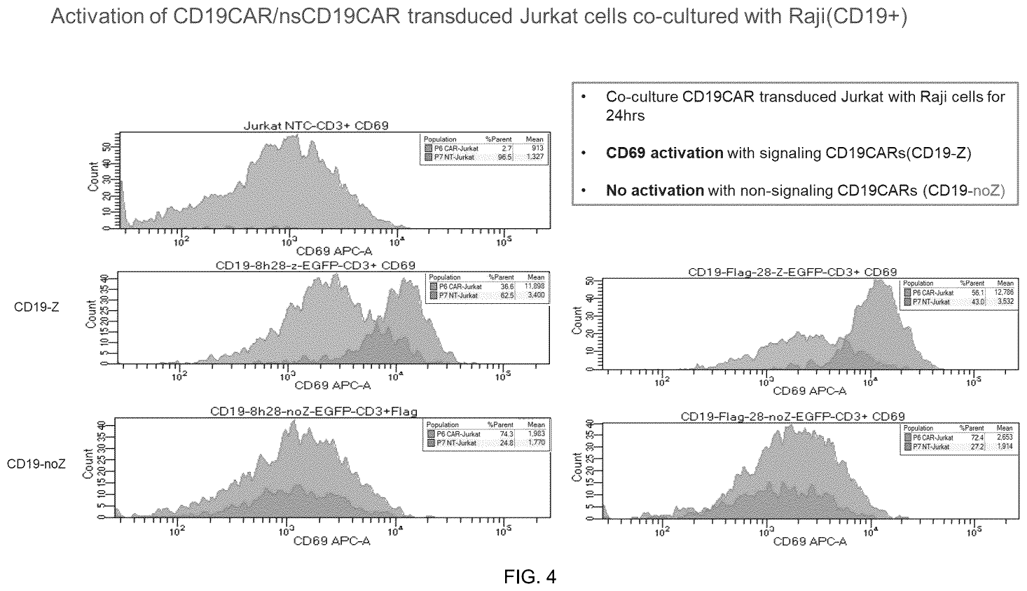

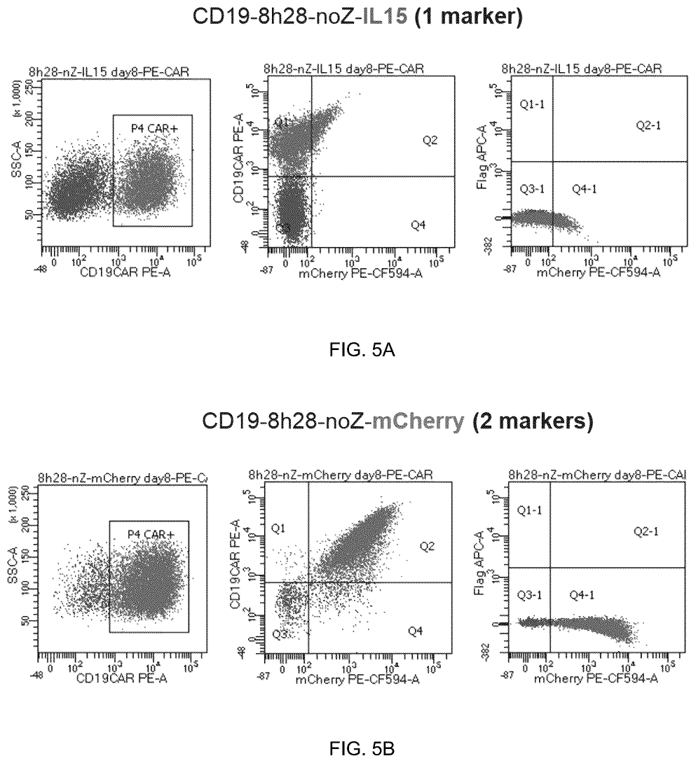

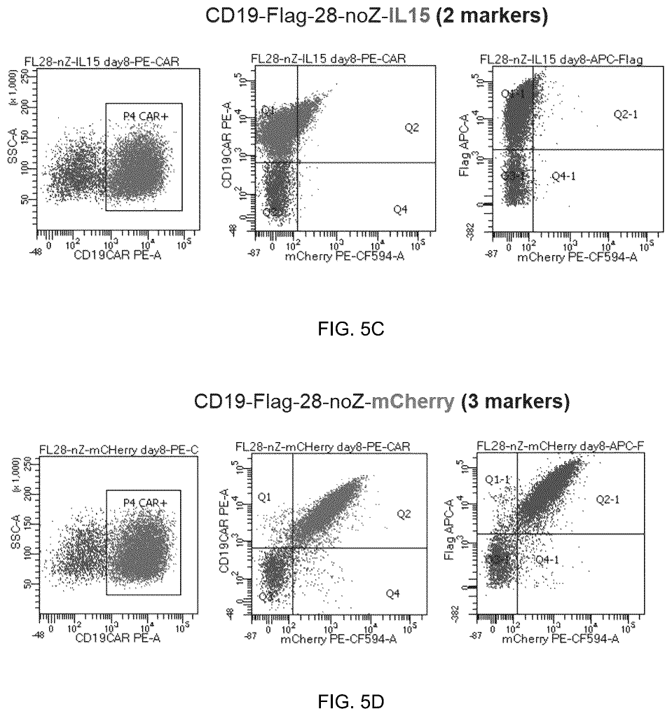

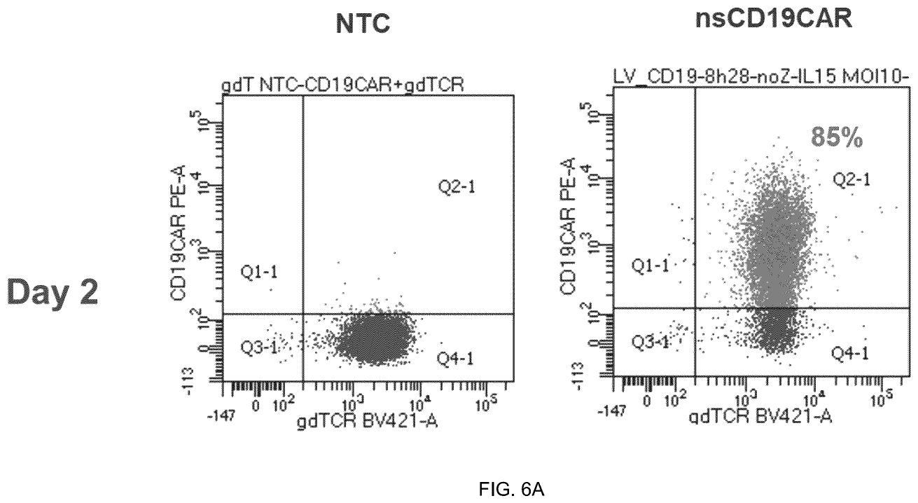

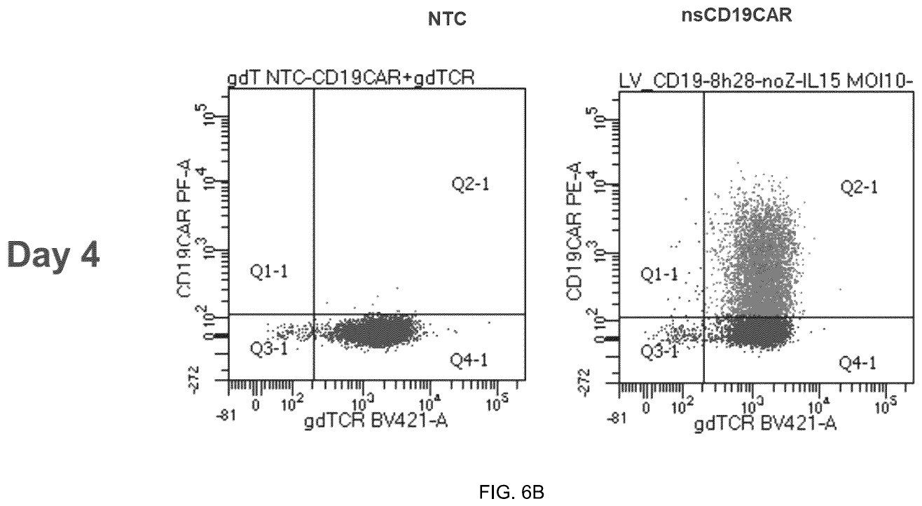

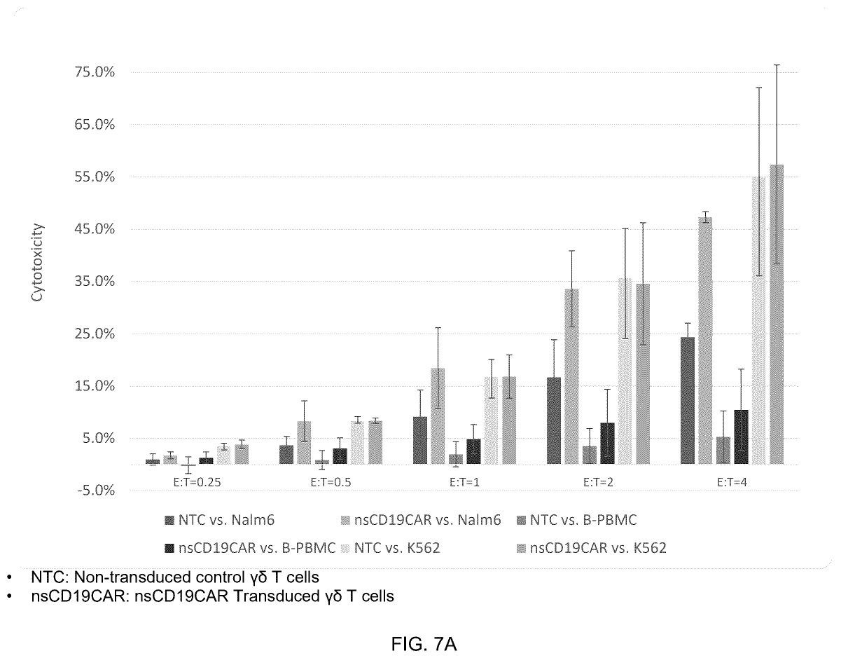

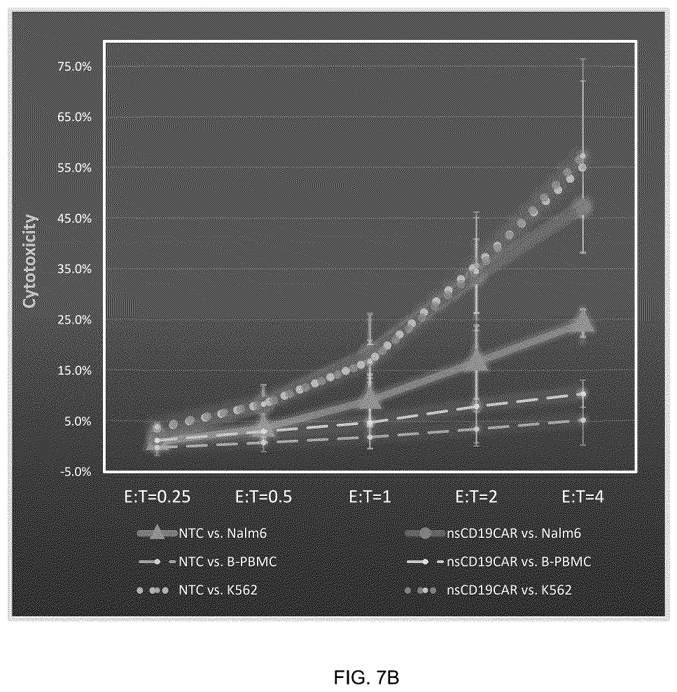

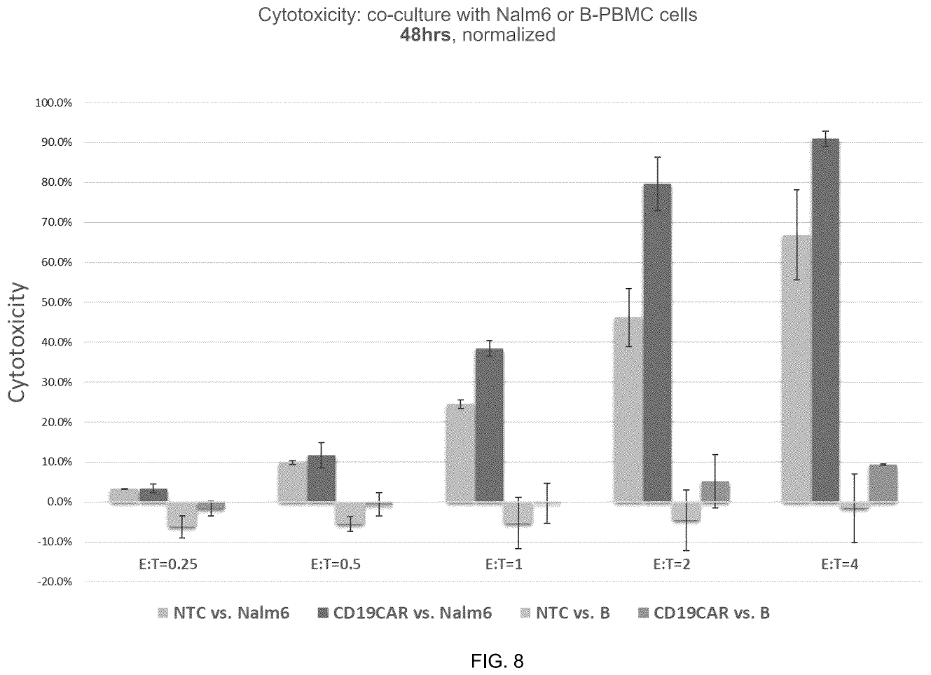

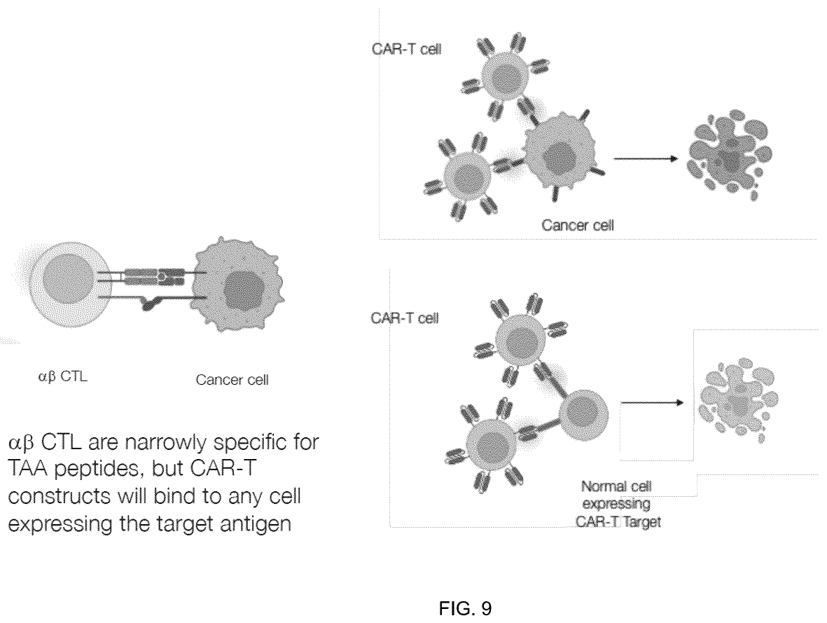

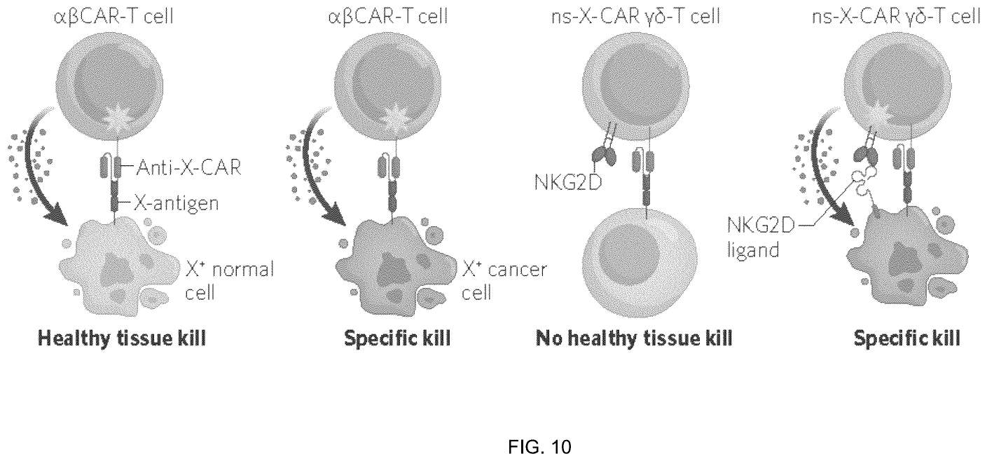

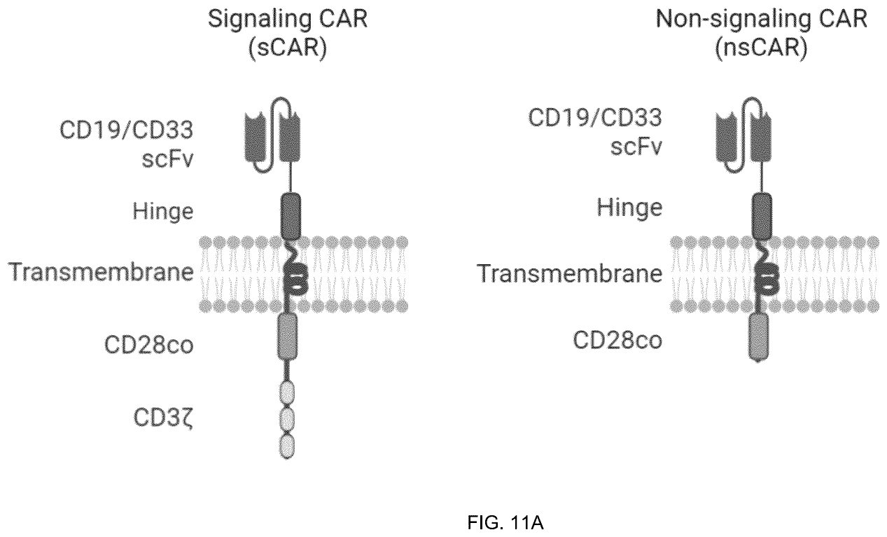

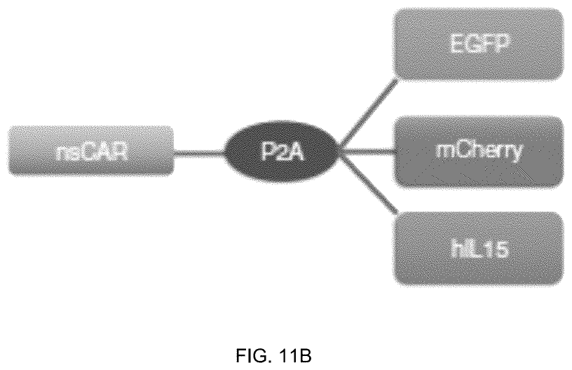

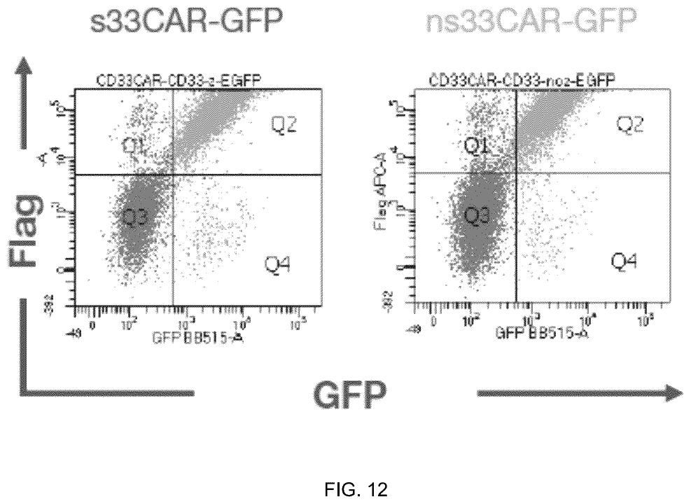

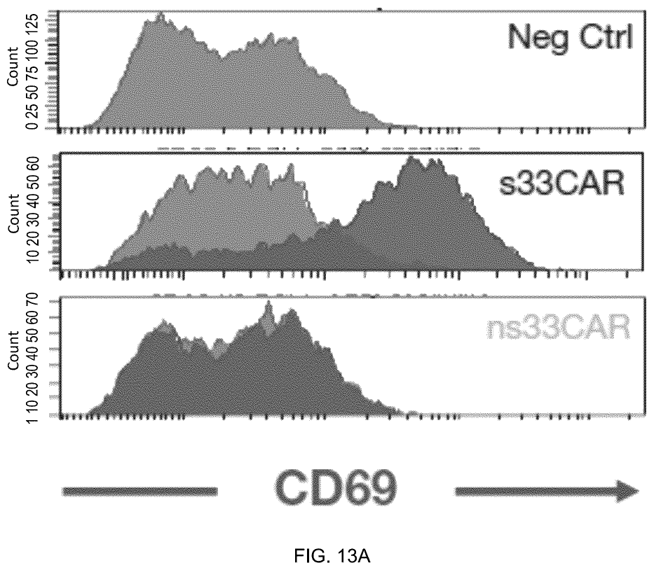

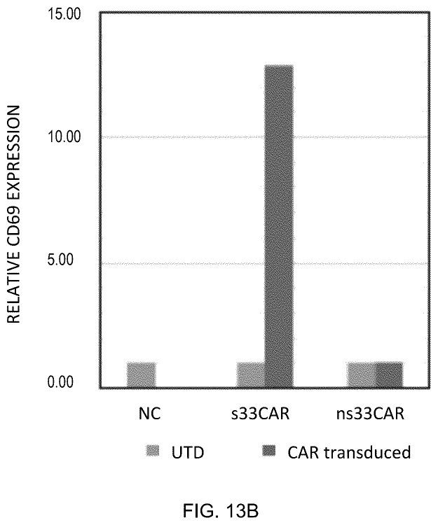

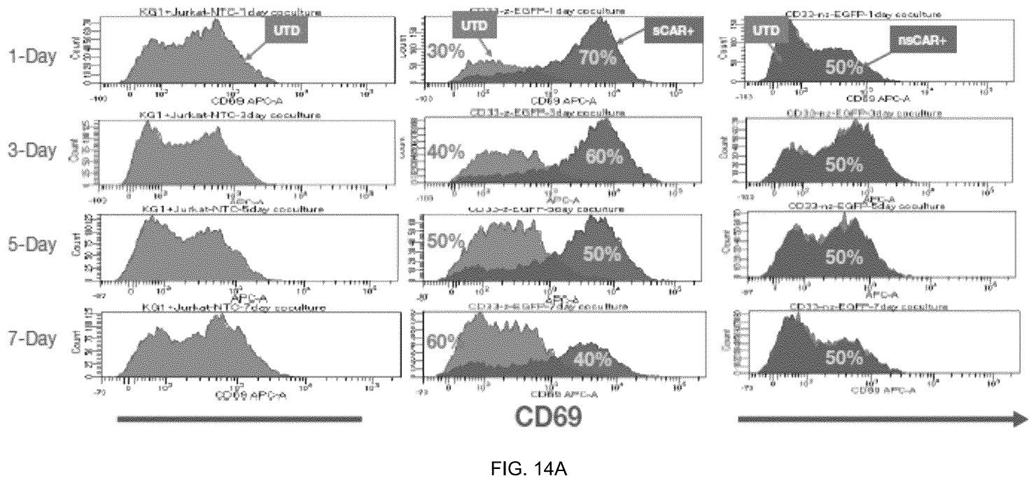

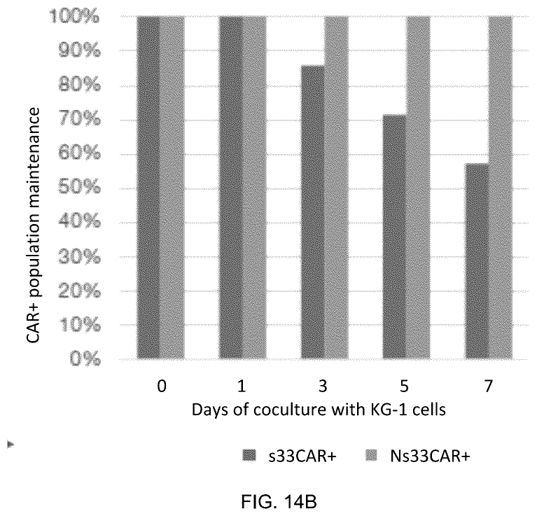

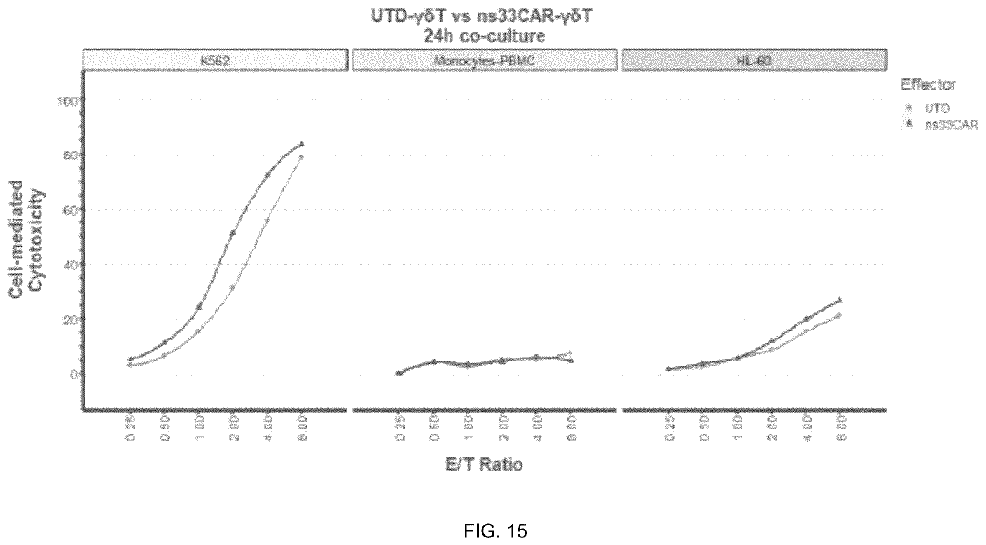

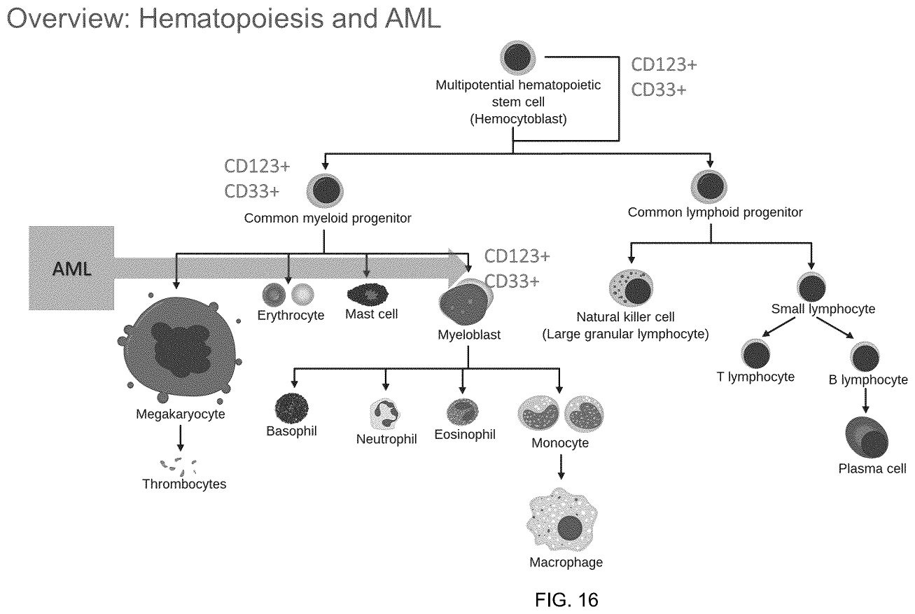

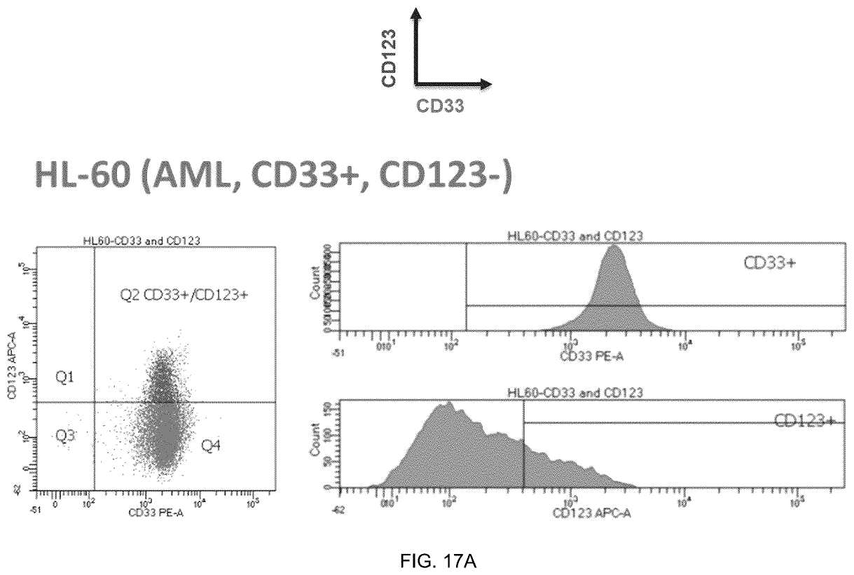

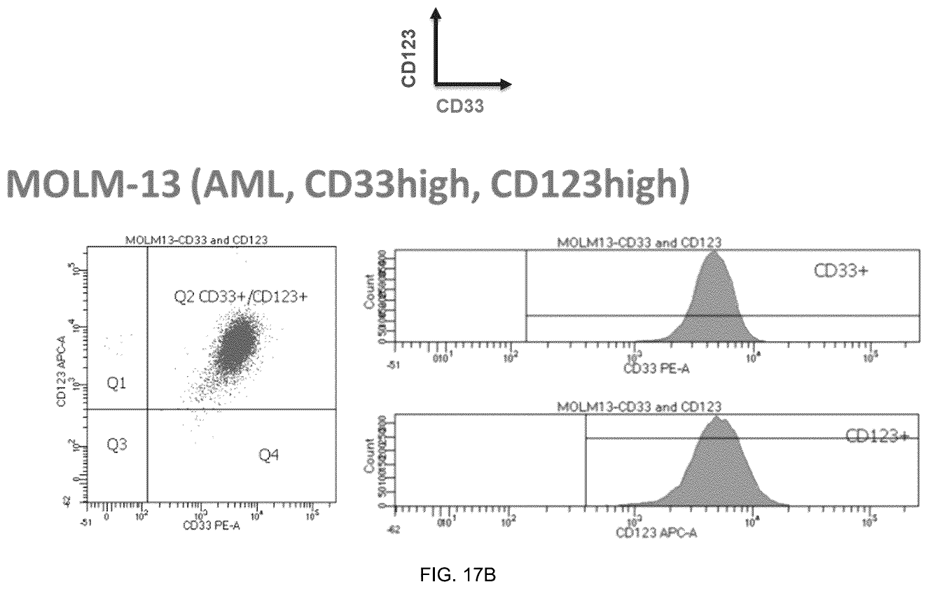

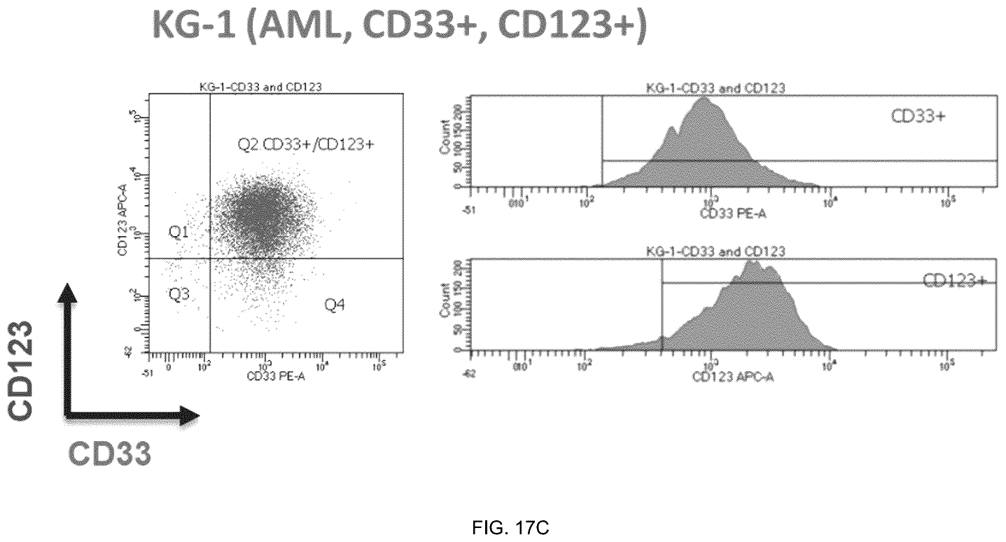

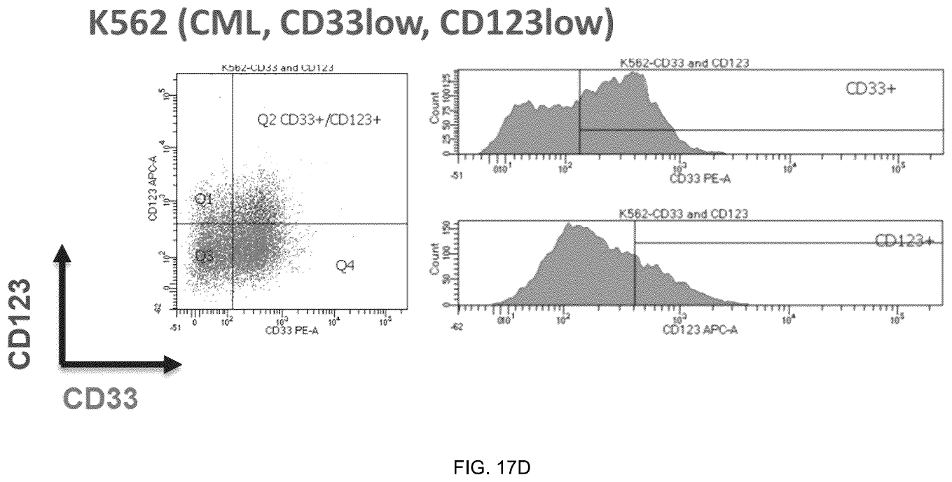

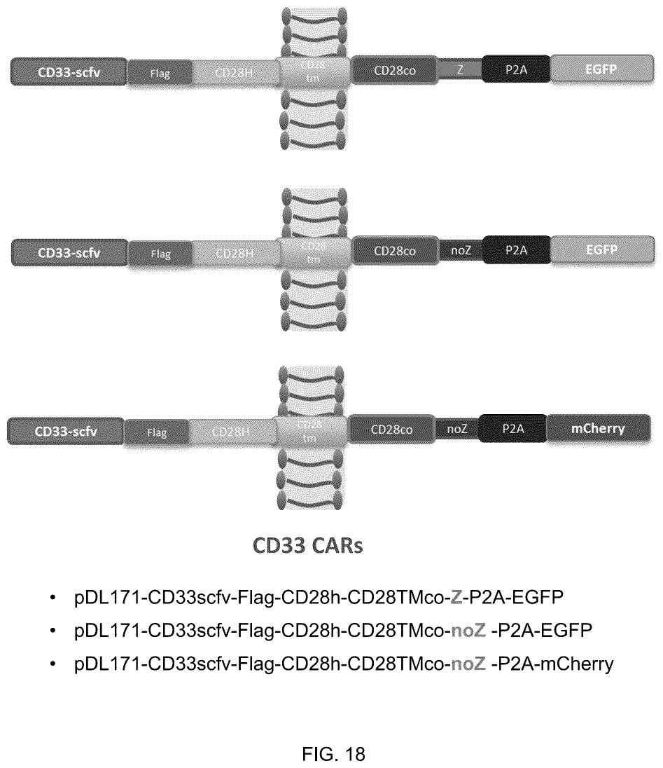

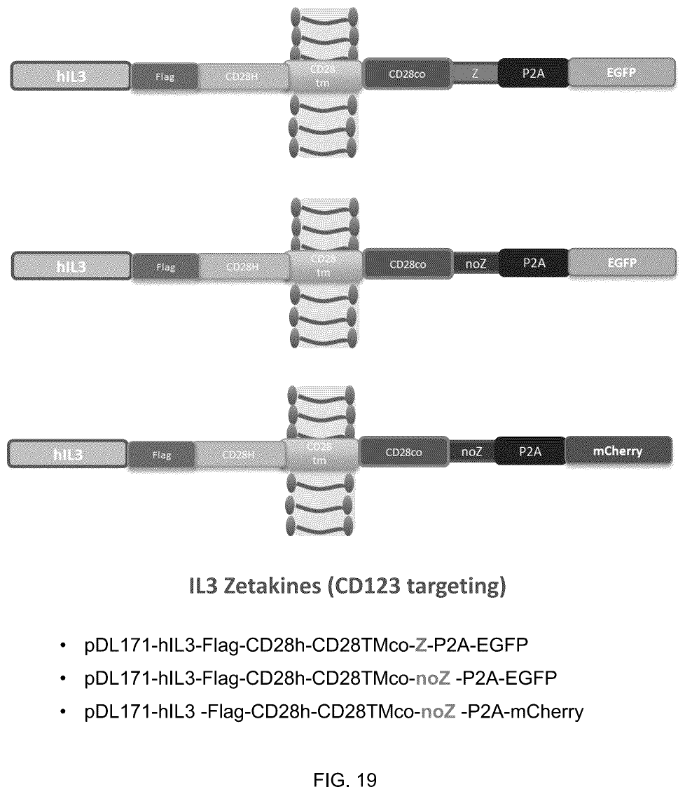

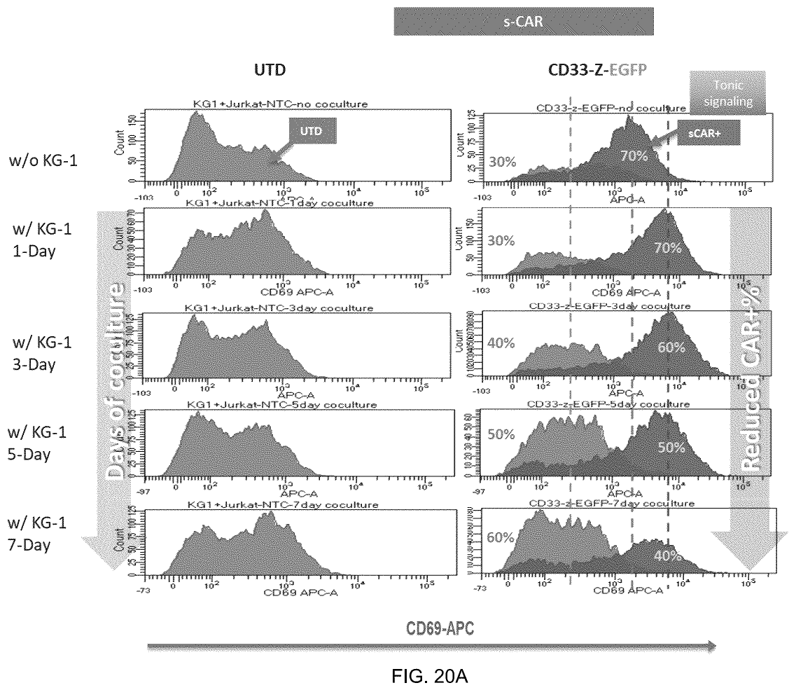

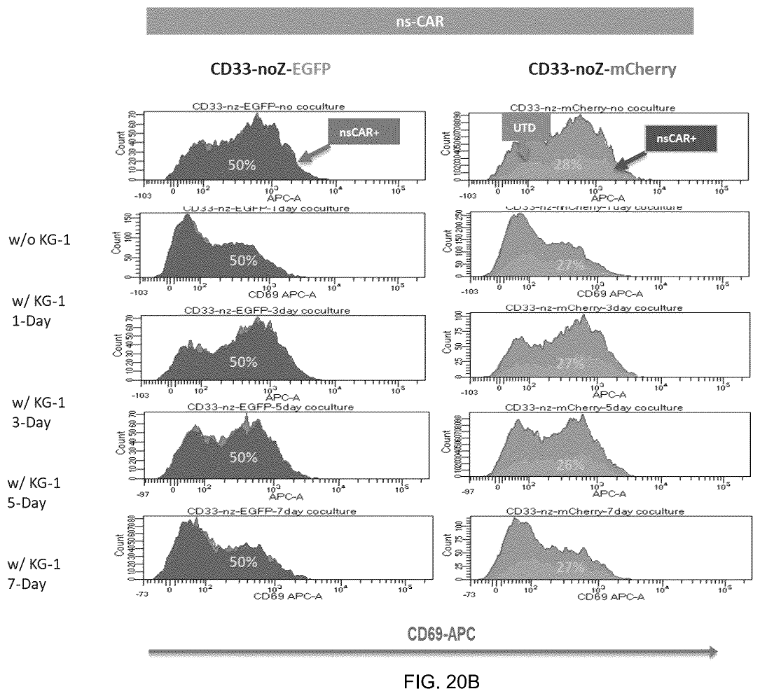

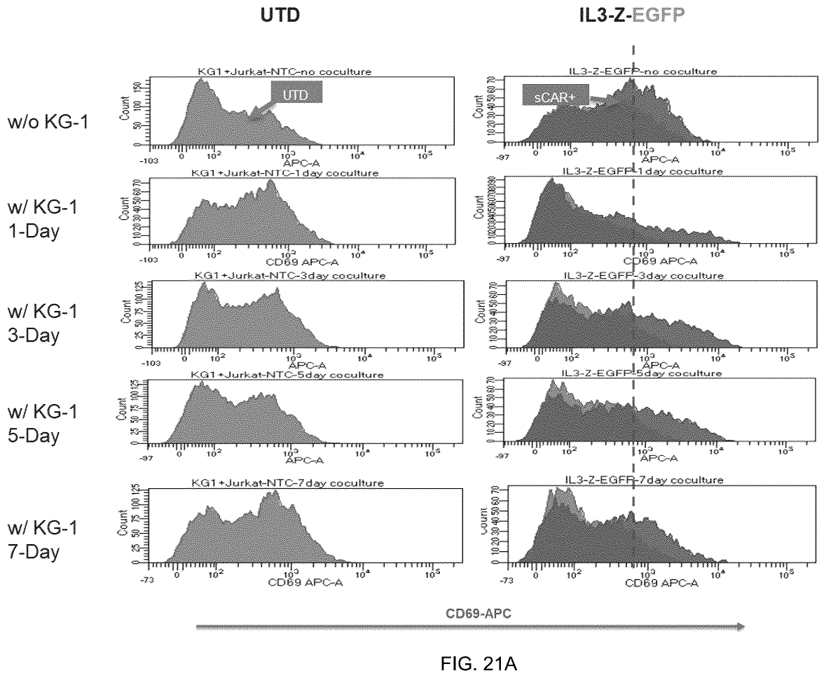

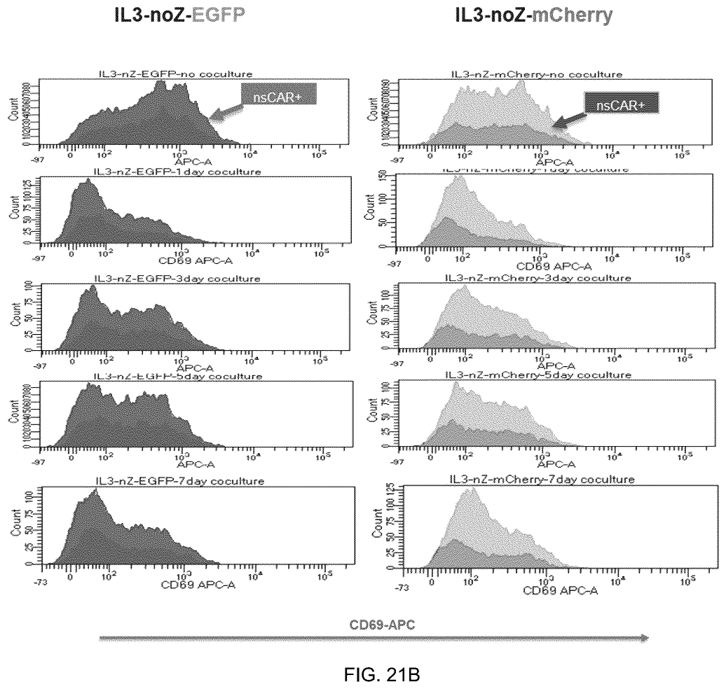

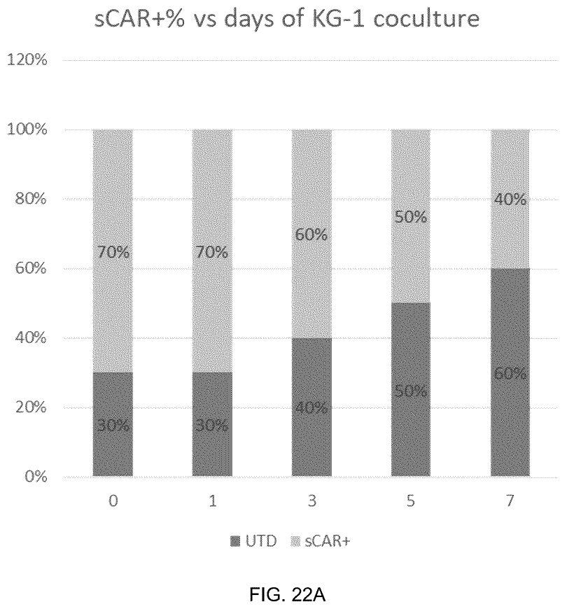

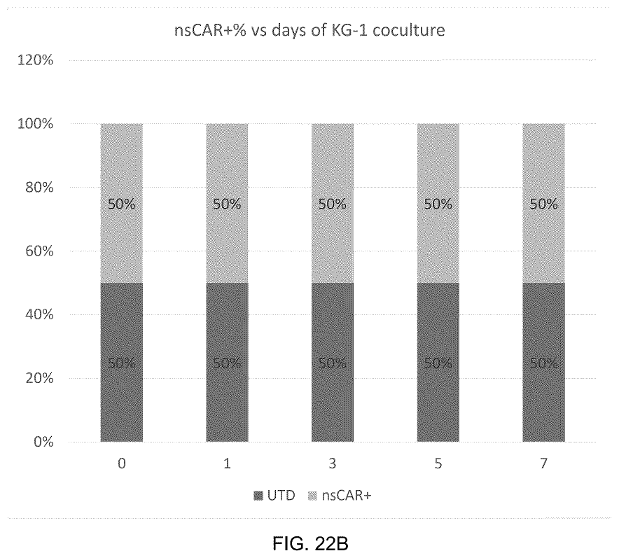

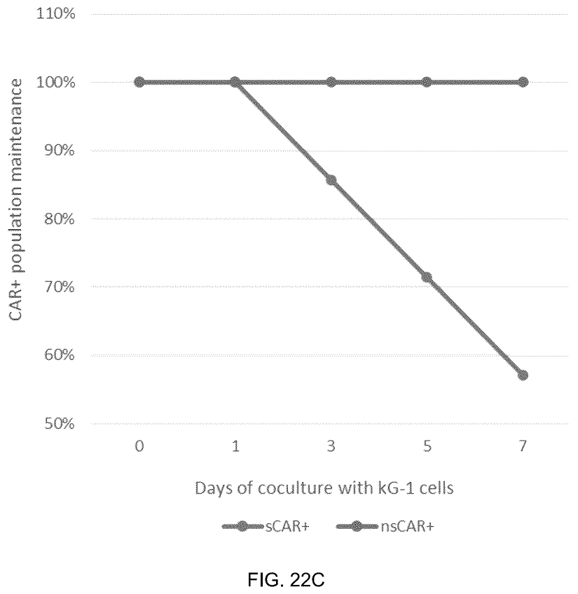

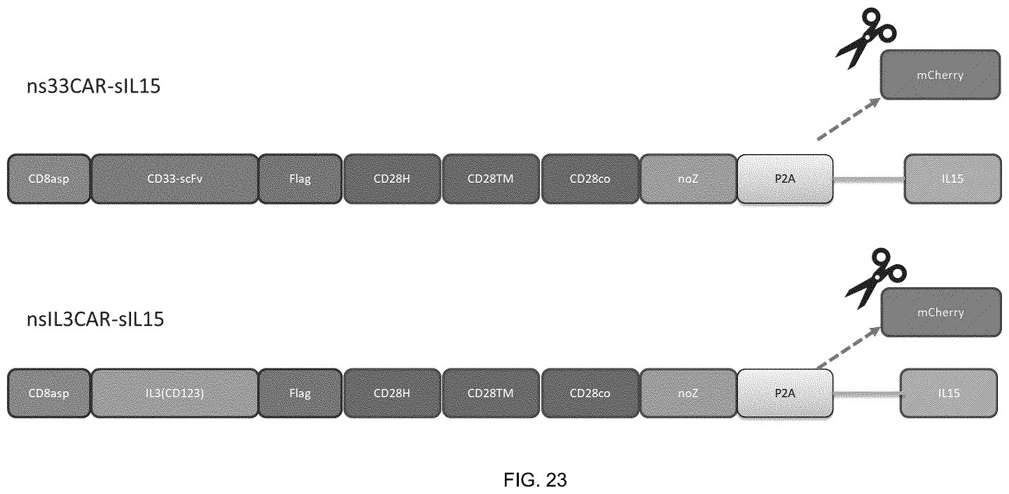

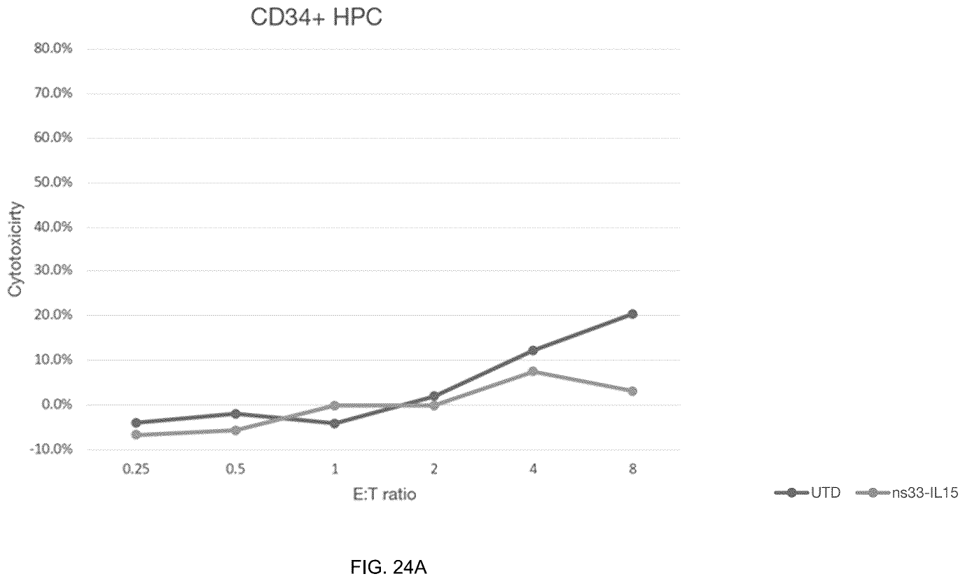

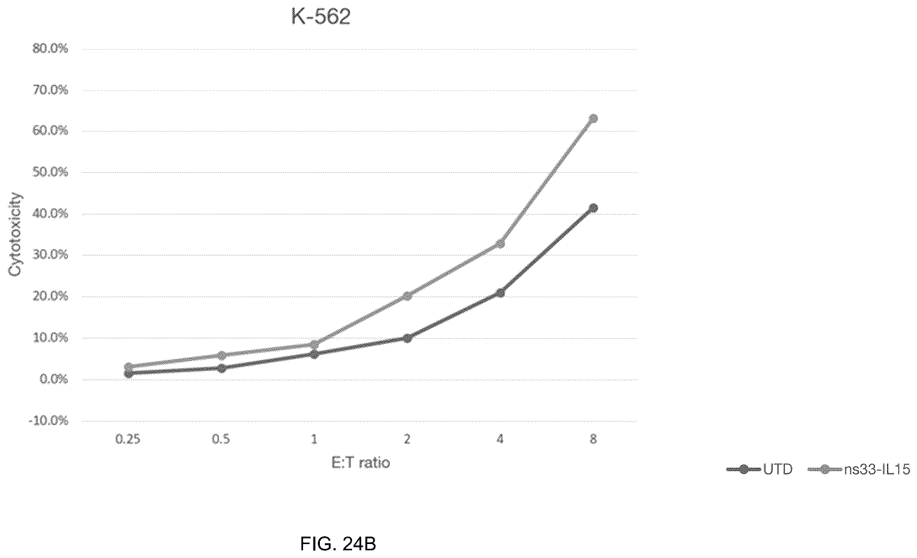

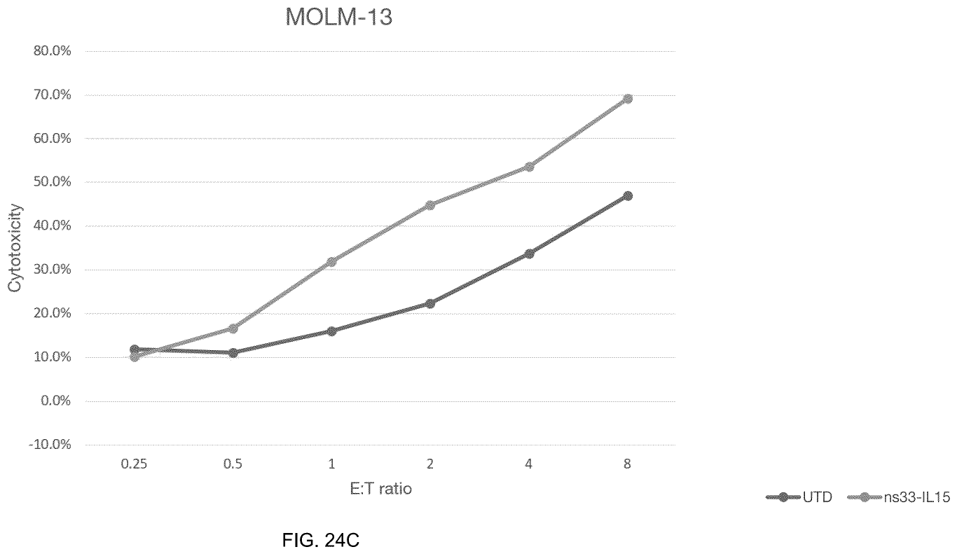

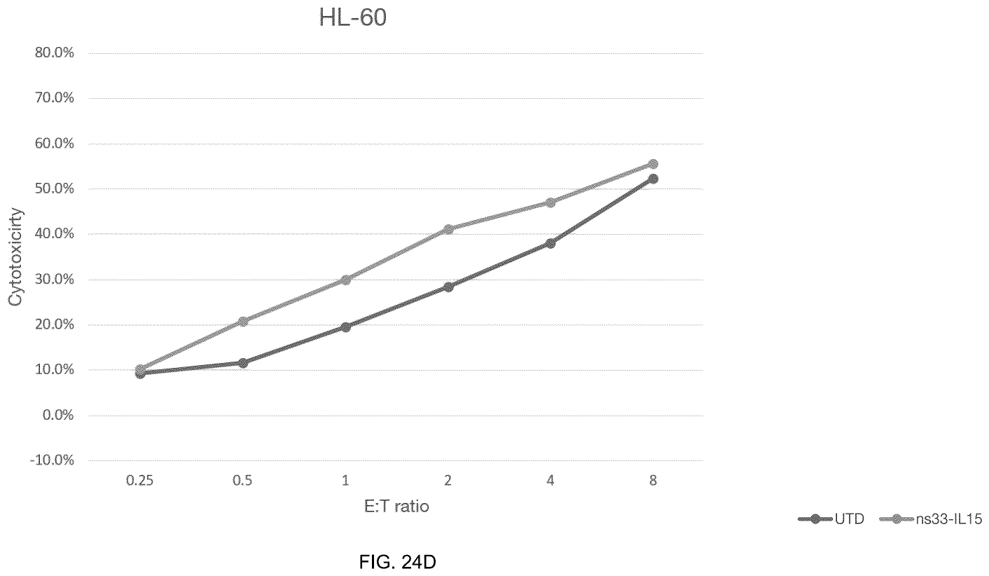

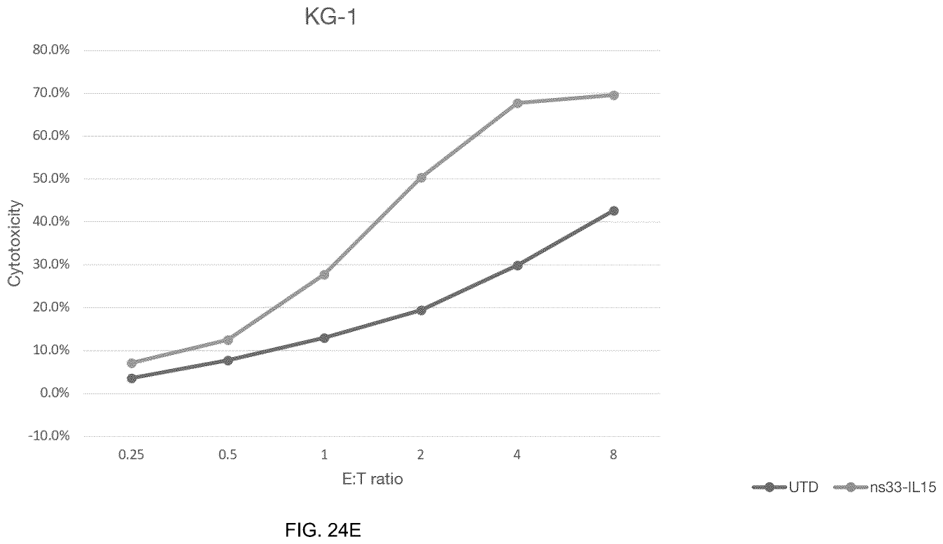

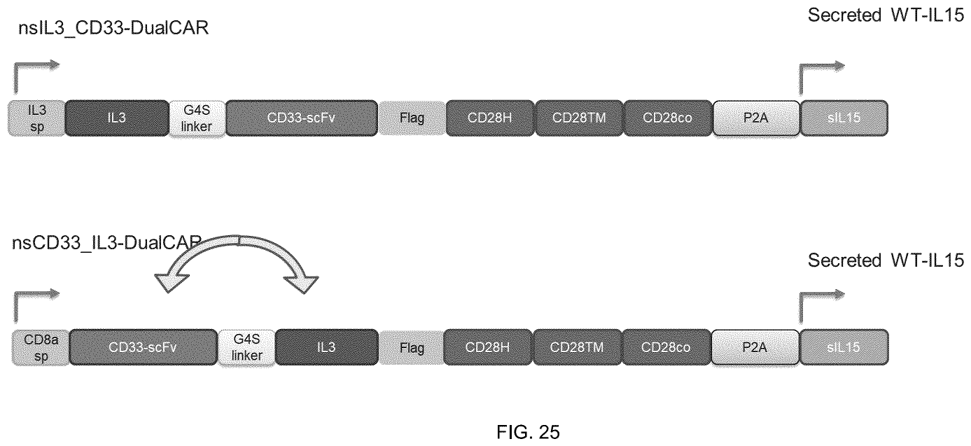

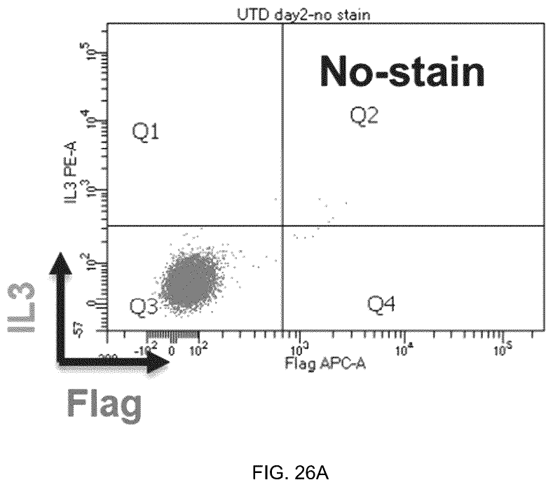

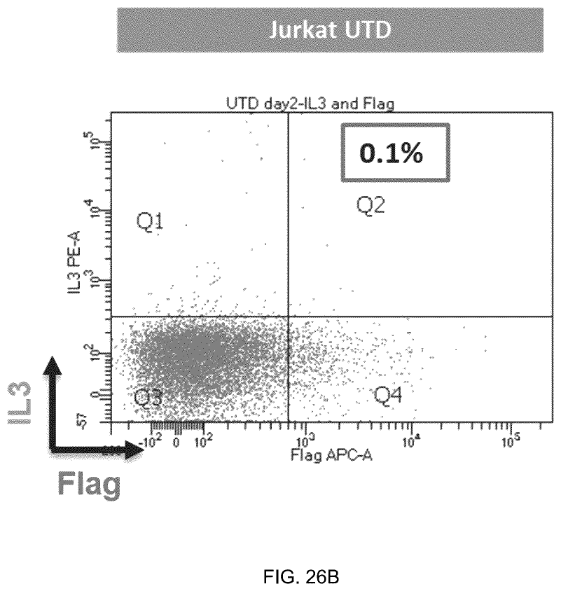

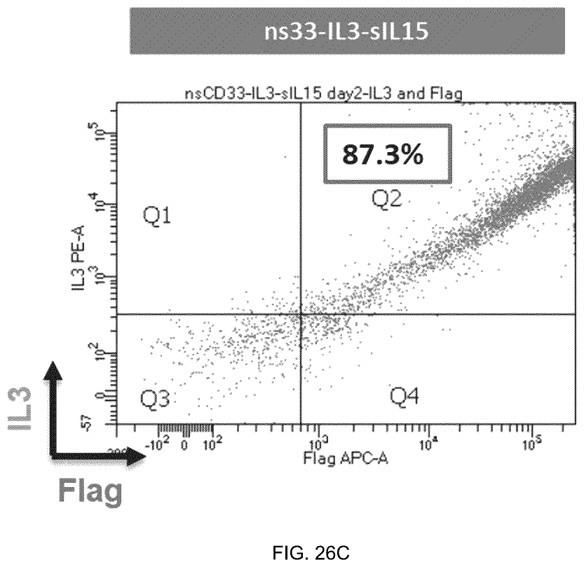

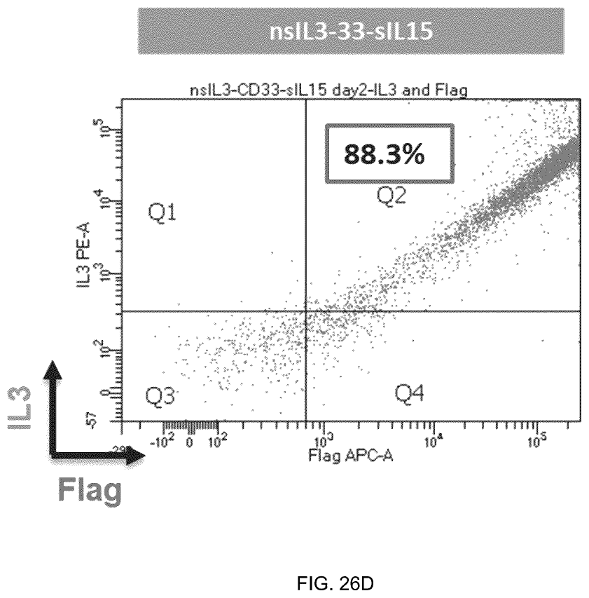

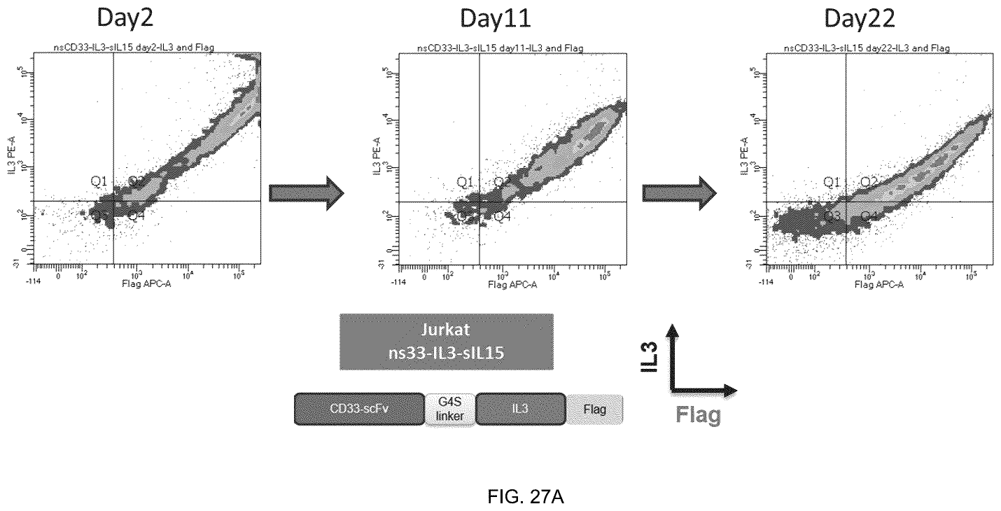

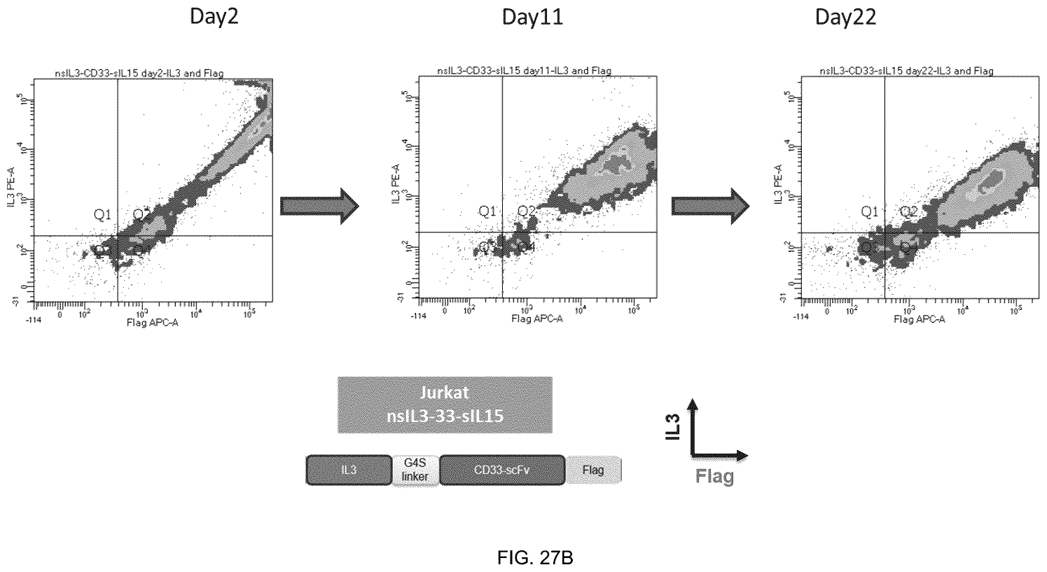

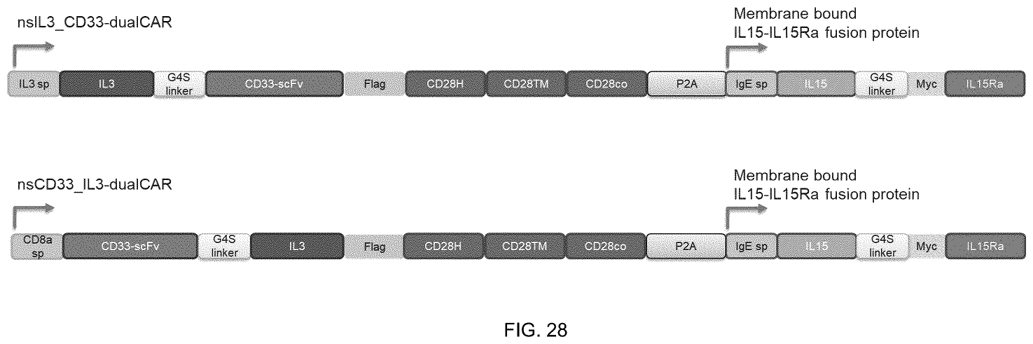

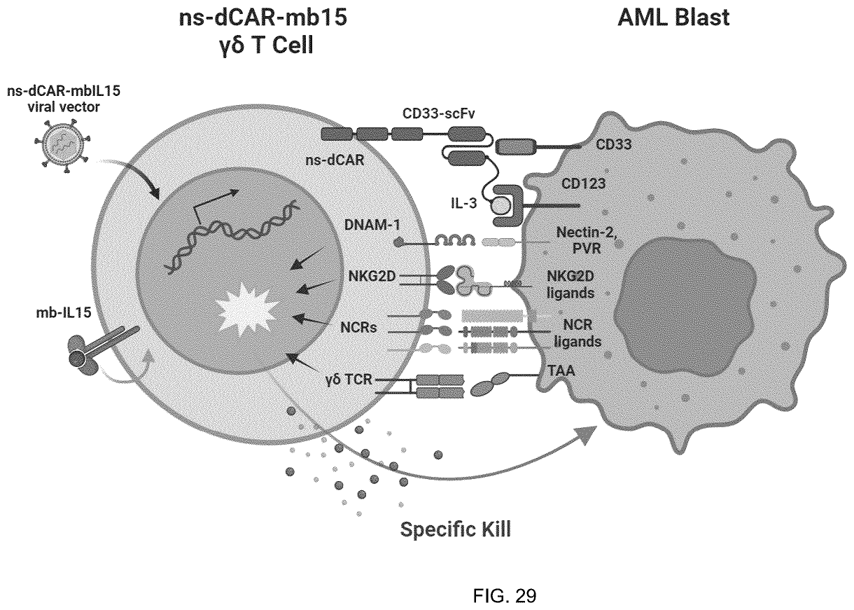

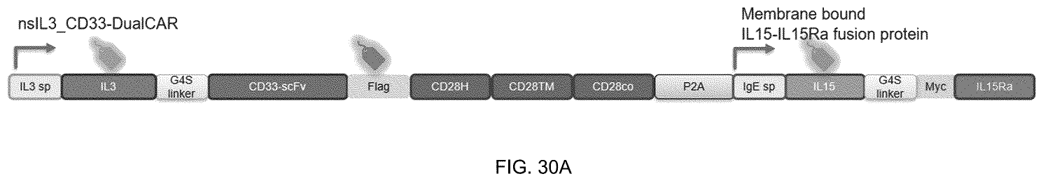

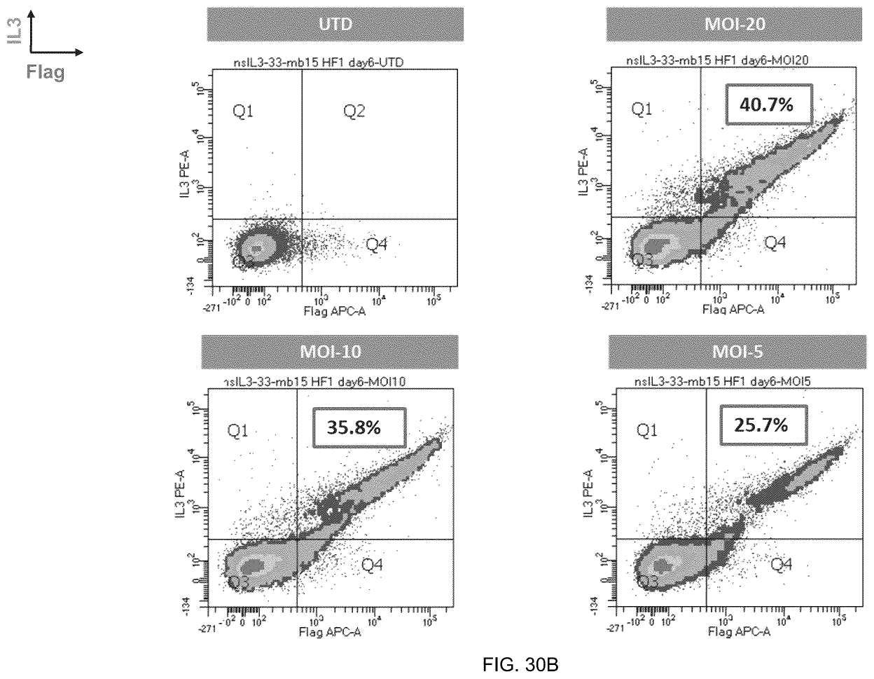

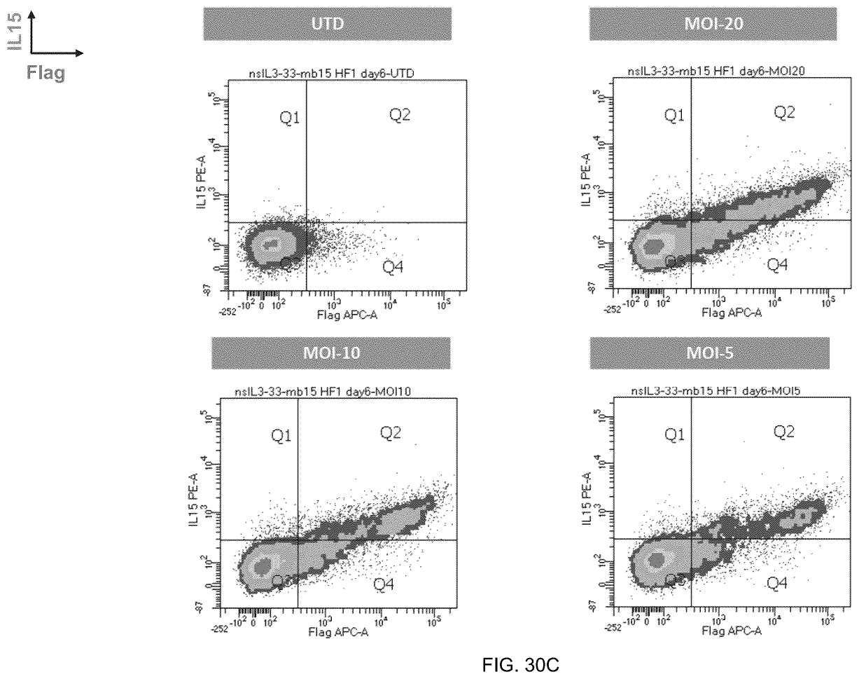

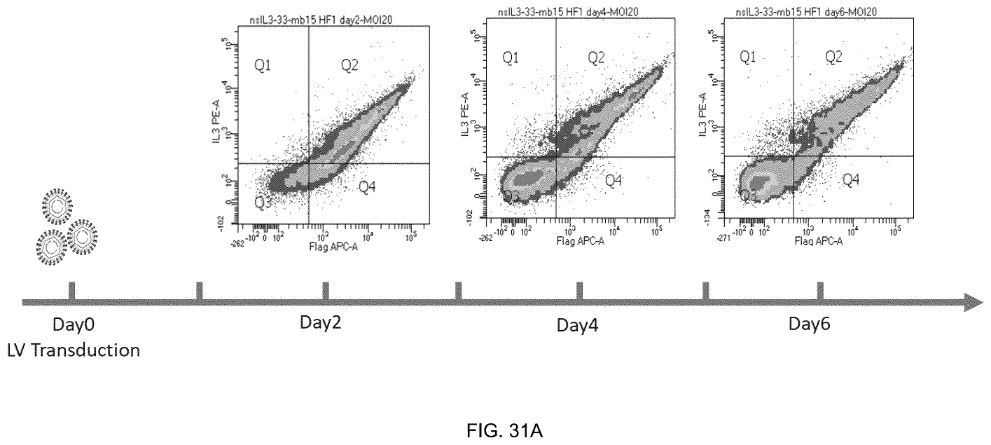

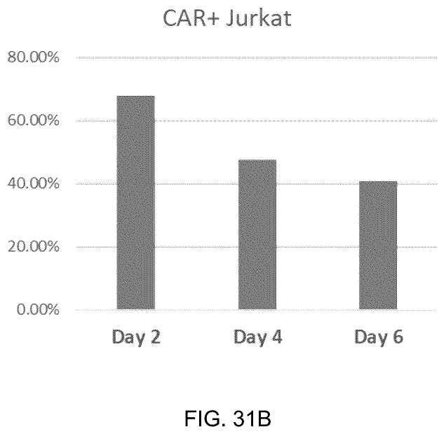

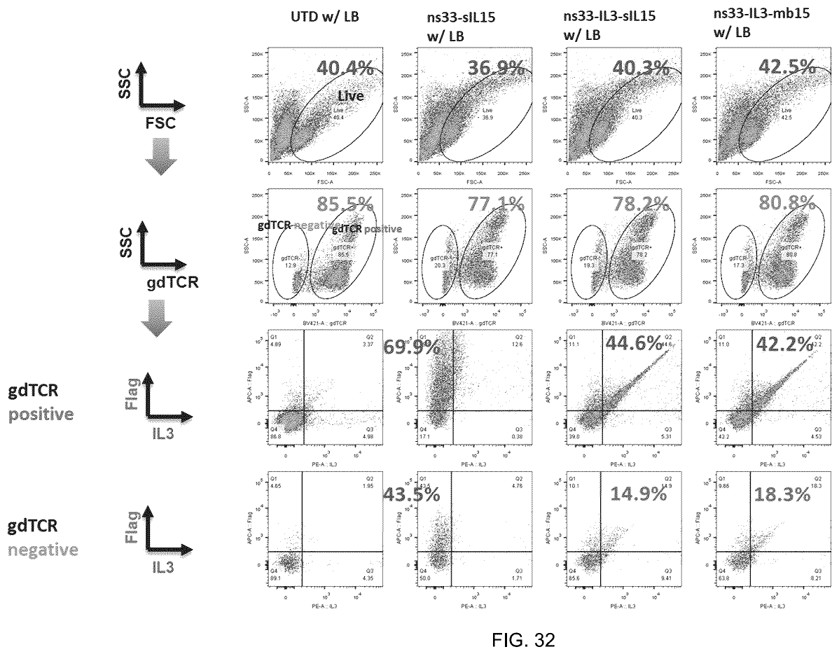

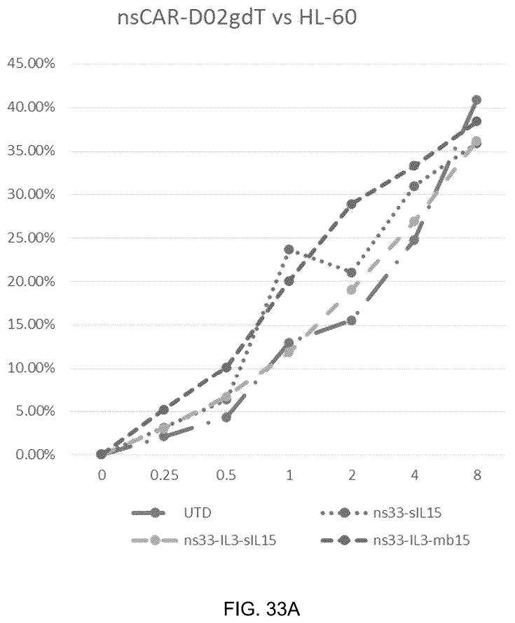

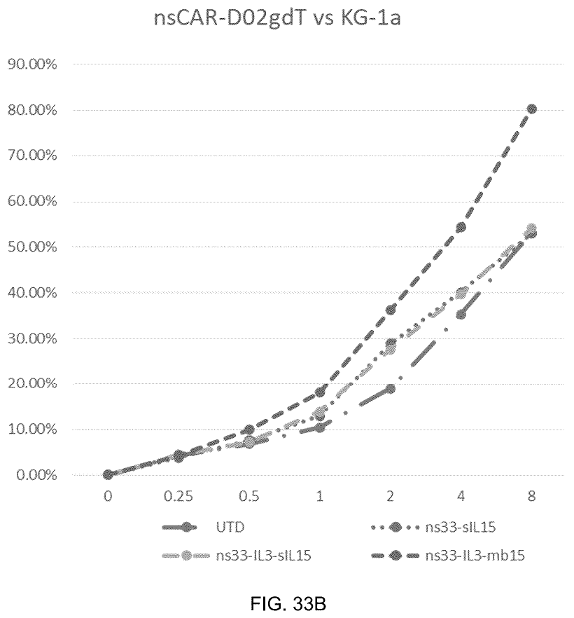

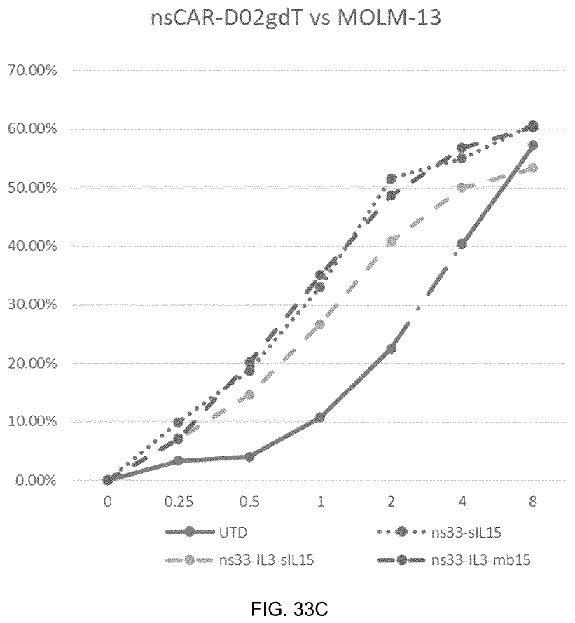

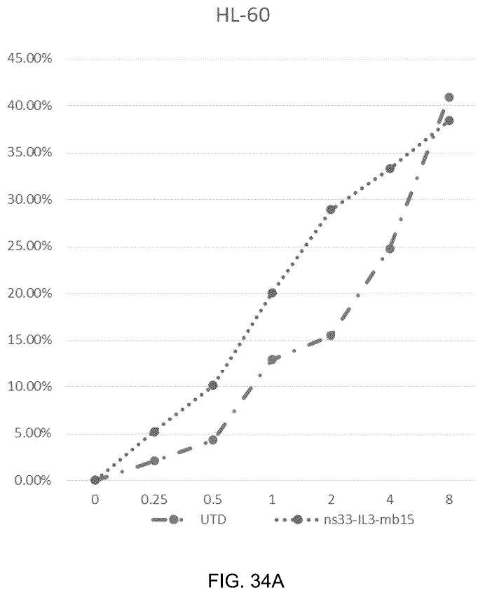

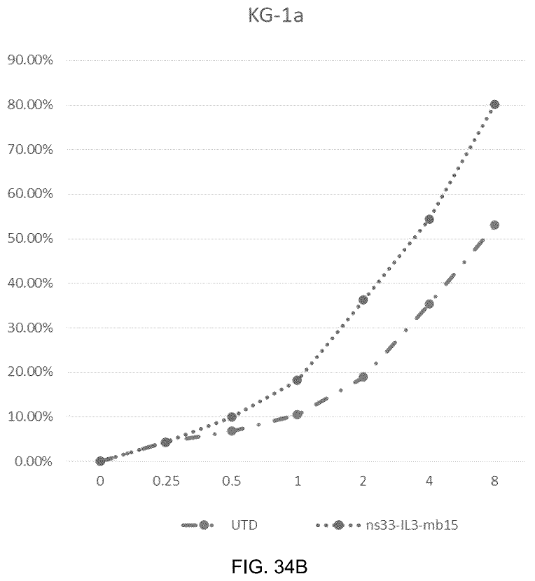

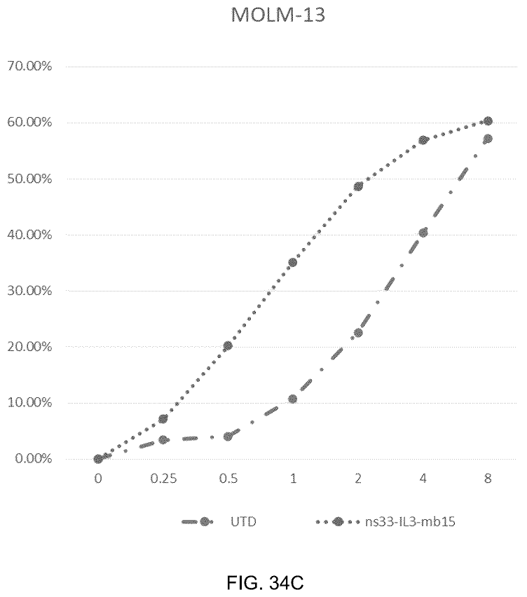

FIG. 2 is a construct map of exemplary anti-CD19 CAR γδ T cell constructs. The constructs can comprise an anti-CD19 scFv and, starting from the left, and one or more of the following additional domains: a CD8 (“CD8H”) or CD28 hinge region (“CD28H”), a CD28 transmembrane domain (“CD28tm”), a CD28 co-stimulatory domain (“CD28co”), a CD3 zeta signaling domain (“CD3C” or “Z”) or no CD3z signaling domain (“noZ”), a P2A peptide, and a co-expressed peptide selected from IL-15, mCherry (a fluorescent protein derived from Discosoma sp.), and eGFP (green fluorescent protein), or a combination thereof. mCherry and eGFP are selectable marker used for research. The depicted schematic includes constructs with the following elements (wherein the left of the construct corresponds to the N-terminal side):
a. anti-CD19 scFv-CD8H-CD28tm-CD28co-Z;
b. anti-CD19 scFv-CD8H-CD28tm-CD28co-Z-IL15;
c. anti-CD19 scFv-CD8H-CD28tm-CD28co-noZ;
d. anti-CD19 scFv-CD8H-CD28tm-CD28co-noZ-IL15;
e. anti-CD19 scFv-CD28H-CD28tm-CD28co-Z;
f. anti-CD19 scFv-CD28H-CD28tm-CD28co-Z-IL15;
g. anti-CD19 scFv-CD28H-CD28tm-CD28co-noZ;
h. anti-CD19 scFv-CD28H-CD28tm-CD28co-noZ-IL15;
i. anti-CD19 scFv-CD8H-CD28tm-CD28co-Z-P2A-mCherry or eGFP;
j. anti-CD19 scFv-CD8H-CD28tm-CD28co-Z-P2A-IL15;
k. anti-CD19 scFv-CD8H-CD28tm-CD28co-noZ-P2A-mCherry or eGFP;
l. anti-CD19 scFv-CD8H-CD28tm-CD28co-noZ-P2A-IL15;
m. anti-CD19 scFv-CD28H-CD28tm-CD28co-Z-P2A-mCherry or eGFP;
n. anti-CD19 scFv-CD28H-CD28tm-CD28co-Z-P2A-IL15;
o. anti-CD19 scFv-CD28H-CD28tm-CD28co-noZ-P2A-mCherry or eGFP; and
p. anti-CD19 scFv-CD28H-CD28tm-CD28co-noZ-P2A-IL15.
   With respect to the constructs described in this disclosure, the invention encompasses constructs where the dash or - between components indicates indirect and/or direct attachment.

FIGS. 3A-3D shows flow cytometric analysis of four lentiviral CD19-CAR constructs and validation in Jurkat T cells; going from left to right, the four lentiviral constructs are:

FIG. 3A: pDL171-FMC63-CD8H-CD28TMco-Z-P2A-EGFP;

FIG. 3B: pDL171-FMC63-CD8H-CD28TMco-noZ-P2A-EGFP;

FIG. 3C: pDL171-FMC63-Flag-CD28H-CD28TMco-Z-P2A-EGFP; and

FIG. 3D: pDL171-FMC63-Flag-CD28H-CD28TMco-noZ-P2A-EGFP “pDL171” is the transfer plasmid for lentiviral packaging. “FMC63” is FMC63-scFv, which sequence was derived from mouse monoclonal antibody for CD19 clone FMC63, an IgG2a mouse monoclonal antibody targeting CD19; “CD28TMco” is the CD28 transmembrane domain and co-stimulatory domain. FIGS. 3A-3D show that Jurkat T cells transduced with the four CD19-CAR lentiviral vectors described above express CD19CAR on their cell surfaces and co-express EGFP inside the cells. The flow cytometry shows CD19-CAR and GFP double positive populations.

FIG. 4 shows flow cytometric analysis of lentivirus transduced Jurkat T cells that were co-cultured with Raji (CD19+) cells for 24 hours and stained with anti-CD69 antibody. Signaling CD19 CAR T-cells (“CD19-Z”) showed potent CD69 activation whereas no activation was seen with non-signaling CD19CARs (“CD19-noZ”).

FIGS. 5A-5D shows flow cytometric analysis of four lentiviral CD19-CAR constructs and validation in Jurkat T cells; the four lentiviral constructs are:

FIG. 5A: pDL171-FMC63-CD8h-CD28TMco-noZ-P2A-IL15 (panel at top left);

FIG. 5B: pDL171-FMC63-CD8h-CD28TMco-noZ-P2A-mCherry (panel at bottom left);

FIG. 5C: pDL171-FMC63-Flag-CD28h-CD28TMco-noZ-P2A-IL15 (panel at top right);
and

FIG. 5D: pDL171-FMC63-Flag-CD28h-CD28TMco-noZ-P2A-mCherry (panel at bottom right).

In FIG. 5A, the only marker in the construct for the transduced cells is CD19-CAR which is detected using an anti-CAR antibody. In FIG. 5B, the construct has two markers, CD19-CAR on the cell surface and mCherry (internal); transduced cells are shown as CAR+/mCherry+. In FIG. 5C, the construct has two markers, CD19-CAR on the cell surface and Flag tag on the cell surface. In FIG. 5C, because the Flag tag and the CD19-CAR are in close-proximity, a single stain was used and the Flag tag is a surrogate marker for the CAR. In FIG. 5D, the construct has three markers, CD19-CAR on the cell surface, Flag tag on the cell surface and mCherry (internal); transduced cells are gated in the CAR/mCherry or Flag/mCherry double positive populations.

FIGS. 6A and 6B shows flow cytometric analysis demonstrating transduction of γδ T cells measured by staining with anti-CD19 CAR monoclonal antibody; “NTC” are non-transduced control γδ T cells and “nsCD19CAR” are non-signaling CD19 CAR transduced γδ T cells. The nsCD19 CAR construct is FMC63-CD8h-CD28TMCo-noZ-IL15.

FIGS. 7A and 7B are graphs showing non-transduced γδ T-cell (NTC) and nsCD19CAR-γδ T cell (non-signaling CD19 CAR) cytotoxicity after 16 hours co-culture with Nalm6 (CD19+) leukemia cells, B-PBMCs (CD19+) and K562 (CD19−) leukemia cells at different effector to target (E:T) ratios. Shown results are normalized from multiple experiments. nsCD19CAR γδ-T cells show greater cytotoxicity against Nalm6 cells than non-transduced control γδ T cells and show little cytotoxicity to B-PBMCs. nsCD19CAR γδ-T cells and non-transduced control γδ T cells show similar cytotoxicity against CD19-K562 cells.

FIG. 8 shows non-transduced γδ T-cell and nsCD19CAR γδ T cell cytotoxicity after 48 hours co-culture with Nalm6 (CD19+) leukemia cells and B-PBMCs (CD19+) at different effector to target (E:T) ratios. nsCD19CAR γδ-T cells show significantly greater cytotoxicity against Nalm6 cells than non-transduced control γδ T cells.

FIG. 9 is a schematic depicting current CAR-T therapies that target tumor-associated antigens expressed on both malignant and normal cells. ap cytotoxic T-lymphocytes (CTLs) are narrowly specific for TAA peptides but CAR T-cell constructs will bind to any cell expressing the target antigen.

FIG. 10 is a schematic showing how nsCAR γδ T cells differentiate between healthy and cancer cells as compared with αρCAR-T cells which do not differentiate between healthy tissue and cancer cells. Activation of the gdT cell is mediated through endogenous γδ T cell receptors and other surface molecules such as NKG2D and DNAM-1 and not through the CAR, allowing this platform to distinguish between healthy and cancer cells.

FIGS. 11A and 11B are schematics showing the design of signaling and non-signaling CAR constructs. FIG. 11A is a schematic showing signaling CAR (sCAR) and non-signaling CAR (nsCAR) domain structures. FIG. 11B is a schematic showing P2A co-expression of genes (EGFP, mCherry and/or human IL15) with nsCAR (e.g, CD19 nsCAR and CD33 nsCAR).

FIG. 12 shows flow cytometric analysis of signaling CD33 CAR (s33CAR) and CD33 nsCAR (ns33CAR) in transduced Jurkat T cells.

FIGS. 13A and 13B show expression of CD69 in CD33 CAR (s33CAR) and CD33 nsCAR (ns33CAR) transduced Jurkat cells after coculture with KG-1 AML cells. FIG. 13A shows flow cytometric analysis of transduced Jurkat T cells that were co-cultured with KG-1 AML cells and stained with anti-CD69 antibody; Jurkat cells transduced with the sCAR show CD69 upregulated expression while Jurkat cells transduced with the nsCAR do not. FIG. 13B is a graph showing relative CD69 expression for negative control (NC), s33CAR and ns33CAR cells.

FIGS. 14A and 14B demonstrate that ns33CAR-Jurkat T cells do now show activation-induced cell death (AICD) after extended co-culture with CD33+KG-1 AML cells. FIG. 14A shows flow cytometric analysis of transduced Jurkat cells and stained with anti-CD69 antibody at 1-day, 3-days, 6-days and 7-days for non-transduced cells (UTD), s33CAR transduced cells and ns33 CAR cells. The population of s33CAR transduced cells decreased from about 70% 1-day after transduction to about 40% at 7 days; in contrast, the population of ns33CAR transduced cells remained about the same over time. FIG. 14B is a graph showing CAR+ population maintenance at 0, 1, 3, 5 and 7 days of co-culture with KG-1 cells for s33CAR+ and ns33CAR+ cells.

FIG. 15 is a graph showing cytotoxicity of non-transduced (UTD) and ns33 CAR γδ T cells against K562 (CD33+) cells, monocyte-PBMCs (CD33+) and HL-60 (CD33+) after 24 hours at different effector: target (E:T) ratios.

FIG. 16 is a schematic that provides an overview of hematopoiesis and the effect of AML. AML is the result of hematopoietic stem or progenitor cell transformation by different genetic mutations and chromosomal rearrangements. These alterations result in clonal expansion of undifferentiated myeloid precursors which results in altered erythropoiesis and bone marrow failure.

FIGS. 17A to 17D shows flow cytometric analysis of CD33 and CD123 expression in AML cell lines, HL-60, MOLM-13, and KG-1 (FIGS. 17A, 17B, and 17C, respectively) and a chronic myeloid leukemia (CML) cell line (K562) (FIG. 17D). HL-60 cells are CD33-positive and CD123-negative. KG-1 cells are positive for both CD33 and CD123. MOLM-13 cells show high expression of CD33 and C123. K562 cells show low expression of CD33 and CD123.

FIG. 18 is a construct map of exemplary anti-CD33 CAR γδ T cell constructs. The constructs can comprise an anti-CD33 scFv and, starting from the left, one or more of the following additional domains: a CD8 hinge region (“CD8H”) or CD28 hinge region (“CD28H”), a CD28 transmembrane domain (“CD28tm” or “CD28TM”), a CD28 co-stimulatory domain (“CD28co”), a CD3 zeta signaling domain (“CD3C” or “Z”) or no CD3z signaling domain (“noZ”), a P2A peptide, and a co-expressed peptide, for example, mCherry (a fluorescent protein derived from Discosoma sp.), and eGFP (green fluorescent protein). mCherry and eGFP/EGFP are selectable marker used for research. The depicted schematic includes constructs with the following elements:
a. anti-CD33 scFv-Flag-CD28H-CD28tm-CD28co-Z-P2A-EGFP;
b. anti-CD33 scFv-Flag-CD28H-CD28tm-CD28co-noZ-P2A-EGFP;
c. anti-CD33 scFv-Flag-CD28H-CD28tm-CD28co-noZ-P2A-mCherry.

FIG. 19 is a construct map of exemplary anti-CD123 CAR γδ T cell constructs. The constructs can comprise a human IL3 (hIL3) which binds CD123, starting from the left, one or more of the following additional domains: a CD8 (“CD8H”) or CD28 hinge region (“CD28H”), a CD28 transmembrane domain (“CD28tm” or “CD28TM”), a CD28 co-stimulatory domain (“CD28co”), a CD3 zeta signaling domain (“CD3C” or “Z”) or no CD3z signaling domain (“noZ”), a P2A peptide, and a co-expressed peptide, for example, mCherry (a fluorescent protein derived from Discosoma sp.), and eGFP (green fluorescent protein). mCherry and eGFP/EGFP are selectable marker used for research. The depicted schematic includes constructs with the following elements:
a. hIL3-Flag-CD28H-CD28tm-CD28co-Z-P2A-EGFP;
b. hIL3-Flag-CD28H-CD28tm-CD28co-noZ-P2A-EGFP;
c. hIL3-Flag-CD28H-CD28tm-CD28co-noZ-P2A-mCherry.

The hIL3 is the full-length wild type human IL3 (Uniprot: P08700; uniprot.org/uniprotkb/P08700/entry #sequences).

FIGS. 20A and 20B demonstrates validation of CD33 sCAR (anti-CD33 CAR with a CD3z signaling domain) and CD33 nsCAR (an anti-CD33 CAR that lacks a CD3z signaling domain), respectively, with CD69 expression in transduced Jurkat T-cells with AML co-culture. The sCAR includes an anti-CD33 scFv, a CD3z signaling domain and EGFP (“CD33-Z-EGFP”) and the nsCARs include an anti-CD33 scFv, no CD3z signaling domain and EGFP or mCherry (“CD33-noZ-EGFP’ and “CD33-noZ-mCherry”) and as further described in FIG. 18. This figure shows flow cytometric analysis of untransduced cells (UTD) and Jurkat T cells transduced with these CARs that were co-cultured with KG-1 AML cells and stained with anti-CD69 antibody for 1, 3, 5 and 7 days as shown. The KG-1 AML cells are CD33+(nTPM:37.9 based on data from proteinatlas.org), CD123+(nTPM: 21.7 based on data from proteinatlas.org) For cells transduced with CD33-Z-EGFP CAR (the sCAR), the population of sCAR+ cells decreases over time; for example the percentage of sCAR+ cells with elevated CD69 expression after 1 day of co-culture with the KG-1 cells was 70% whereas the percentage of sCAR+ cells after 7-days of co-culture with the KG-1 cells is 40%. In contrast, for the nsCAR cells (CD33-noZ-EGFP and CD33-noZ-mCherry), there is no significant change in the percentage of CAR-positive cells over time.

FIGS. 21A and 21B demonstrates validation of IL3 zetakine CAR sCAR (IL3 zetakine CAR with a signaling domain) and IL3 zetakine CAR nsCARs (IL3 zetakine CARs that lacks a CD3z signaling domain), respectively, with CD69 activation in transduced Jurkat T-cells with AML co-culture. IL3-CAR Jurkat cells were co-cultured with KG-1 AML cells (CD33+/CD123+) for up to 7 days and stained for CD69. The sCAR includes an IL3, a CD3z signaling domain and EGFP (“CD33-Z-EGFP”) and the nsCARs include an IL3, no CD3z signaling domain and EGFP or mCherry (“CD33-noZ-EGFP’ and “CD33-noZ-mCherry”) and as further described in FIG. 19. Untransduced cells (UTD) and Jurkat T cells transduced with these CARs were co-cultured with KG-1 AML cells and stained with anti-CD69 antibody for 1, 3, 5 and 7 days as shown. For the sCARs, Overall, for the IL3 sCARs, less pronounced CD69 expression after KG-1 coculture is observed as compared to the CD33 sCAR cells in FIG. 20, and little or no change in the percentage of CAR-positive cells was over time was observed.

FIG. 22A-22C are graphs of the flow cytometric data shown in FIG. 20 and show that nsCD33 CAR (anti-CD33 CAR that lacks a CD3z signaling domain) transduced Jurkat cells do now show AICD after extended co-culture with CD33+KG-1 AML cells. Specifically, FIG. 22A shows that for the sCARs, the percentage CAR-positive cells was reduced over time. In contrast, FIG. 22B shows that for cells transduced with the nsCAR, the percentage of CAR-positive cells remained the same over time. FIG. 22C is a graph comparing CAR+ cell population maintenance over time (days of co-culture with KG-1 cells) and demonstrates that nsCARs do not show activation induced cell death (AICD) in contrast to the sCAR expressing gdT cells which show evidence of a decreasing population likely due to AICD.

FIG. 23 shows two construct maps of exemplary non-signaling anti-CD33 and IL3 zetakine CARs (CD33/CD123 dualCAR) which co-express IL-15 (secreted wild-type human IL-15; “sIL15”). The constructs can comprise an anti-CD33 scFv and, starting from the top left, one or more of the following additional domains: a CD8a signal peptide (CD8asp), a CD8 (“CD8H”) or CD28 hinge region (“CD28H”), a CD28 transmembrane domain (“CD28tm” or “CD28TM”), a CD28 co-stimulatory domain (“CD28co”), no CD3z signaling domain (“noZ”), a P2A peptide, IL15 (secreted IL15), and a co-expressed peptide, for example, mCherry (a fluorescent protein derived from Discosoma sp.), and eGFP (green fluorescent protein). The depicted schematic is a construct with the following elements: CD8asp-anti-CD33 scFv-Flag-CD28H-CD28tm-CD28co-noZ-P2A-sIL15 (“ns33CAR-sIL15”). Starting at the bottom left, the second construct can comprise CD8asp, a human IL3 (IL3) and one or more of the following additional domains: a CD8a signal peptide (CD8asp), a CD8 (“CD8H”) or CD28 hinge region (“CD28H”), a CD28 transmembrane domain (“CD28tm” or “CD28TM”), a CD28 co-stimulatory domain (“CD28co”), no CD3z signaling domain (“noZ”), a P2A peptide, and a co-expressed peptide, for example, mCherry (a fluorescent protein derived from Discosoma sp.), and eGFP (green fluorescent protein). mCherry and eGFP/EGFP are selectable marker used for research. The depicted schematic is a construct with the following elements): CD8asp-IL3-Flag-CD28H-CD28tm-CD28co-noZ-P2A-sIL15 (“nsIL3CAR-sIL15”).

FIG. 24A-24E shows cytotoxicity of nsCD33CAR γδ T cells that co-express sIL15 (nsCAR33CAR-sIL15 γδ T cells; CAR construct described in FIG. 23) after 24 hours co-culture with AML cell lines (MOLM-13, HL-60, and KG-1), K562 cells, and normal CD34+ hematopoietic progenitor cells (HPCs) at effector to target (E:T) ratios of 0.25, 0.5, 1, 2, 4 and 8.

FIG. 24A is a graph showing cytotoxicity (as percent cytotoxicity) of untransduced γδ T cells (UTD) and nsCD33CAR γδ T cells against CD34+ HPCs at the different effector:target (E:T) ratios; the untransduced cells and the nsCAR transduced γδ T cells show no or very low cytotoxicity to the CD34+ HPCs. FIG. 24B shows the cytotoxicity (percent cytotoxicity) of untransduced γδ T cells and nsCD33CAR γδ T cells against K-562 cells at the different effector: target (E:T) ratios. FIGS. 24C-24E shows cytotoxicity (percent cytotoxicity) of untransduced γδ T cells and nsCD33CAR γδ T cells against AML cell lines. These figures show that nsCD33 CAR γδ T cells have greater cytotoxicity against K-562 and AML cell lines than untransduced control γδ T cells, and show little toxicity to healthy donor bone marrow CD34+ HPCs.

FIG. 25 shows construct maps of exemplary non-signaling CD33/CD123 tandem dual-target CARs which co-express secreted IL-15 (sIL15). The constructs can comprise an anti-CD33 scFv and IL3, and starting from the left, one or more of the following additional domains: an IL3 signal peptide (IL3sp) or a CD8a signal peptide (CD8asp), a CD8 (“CD8H”) or CD28 hinge region (“CD28H”), a CD28 transmembrane domain (“CD28tm” or “CD28TM”), a CD28 co-stimulatory domain (“CD28co”), no CD3z signaling domain (“noZ”/not shown), a P2A peptide, secreted IL15 (sIL15), and a co-expressed peptide, for example, mCherry (a fluorescent protein derived from Discosoma sp.), and eGFP (green fluorescent protein). The anti-CD33 scFv can be linked with a peptide linker such GGGGS (“G4S linker”). The top depicted schematic shows a construct with the following elements: IL3sp-IL3-G4S linker-CD33 scFv-Flag-CD28H-CD28tm-CD28co-P2A-sIL15 (“nsIL3-33-sIL15”). The bottom depicted schematic shows a construct with the following elements: CD8asp-CD33scFv-G4S linker-IL3-Flag-CD28H-CD28tm-CD28co-P2A-sIL15 (“ns33-IL3-sIL15”). The figure shows that the ns33-IL3-sIL15 construct differs from the nsIL3-33-sIL15 construct with respect to the order of the anti-CD33 scFv and IL3 domains relative to the N-terminus. In nsIL3_CD33-Dual CAR, the IL3 is closer to the N-terminus and linked to the anti-CD33 scFv via a linker. In nsCD33_IL3-Dual CAR, the anti-CD33 scFv is closer to the N-terminus and linked to the IL3 via the linker.

FIGS. 26A-26D shows flow cytometric analysis of the nsCARs described in FIG. 25 in transduced Jurkat T cells. Jurkat UTD are untransduced Jurkat cells. FIGS. 26B-26D shows flow cytometric analysis of IL3/Flag expression conducted 2 days after transduction for untransduced Jurkat cells (FIG. 26B), ns33-IL3-sIL15 CAR transduced cells (FIG. 26C) and nsIL3-33-sIL15 CAR transduced cells (FIG. 26D). FIG. 26A shows flow cytometric analysis for untransduced cells with no stain. FIG. 26C-26D shows that Jurkat cells were transduced with the non-signaling dual CARs (ns-dCARs), ns33-IL3-sIL15 CAR and nsIL3-33-sIL15 CAR.

FIGS. 27A and 27B show flow cytometric analysis for CAR expression over time. Specifically, flow cytometric analysis of IL3/Flag expression was conducted in Jurkat cells transduced with ns33-IL3-sIL15 (FIG. 27A) and nsIL3-33-sIL15 CAR (FIG. 27B) at Day 2, Day 11 and Day 22 after transduction. The constructs are shown in the figure and described in more detail in the description of FIG. 25. Both constructs demonstrate stable dual CAR expression with the nsIL3-33-sIL15 CAR Jurkat cells showing higher mean fluorescence intensity (MFI).

FIG. 28 shows construct maps of exemplary non-signaling CD33/CD123 dual-target CARs which co-express membrane-bound IL-15-IL15ra fusion protein (described, for example, in Hurton et al. (2014), “TETHERED IL-15 TO AUGMENT THE THERAPEUTIC POTENTIAL OF T CELLS EXPRESSING CHIMERIC ANTIGEN RECEPTOR: MAINTAINING MEMORY POTENTIAL, PERSISTENCE, AND ANTITUMOR ACTIVITY” (2014). The University of Texas MD Anderson Cancer Center UTHealth Graduate School of Biomedical Sciences Dissertations and Theses (Open Access). digitalcommons.library.tmc.edu/utgsbs_dissertations/421; the contents of which are expressly incorporated by reference herein). The constructs can comprise an anti-CD33 scFv and IL3 and membrane-bound IL15-IL15Ra fusion protein (“membrane bound IL15” or “mbIL15”), and further comprising one or more of the following additional domains: an IL3 signal peptide (IL3sp) or a CD8 signal peptide (CD8asp), a CD8 (“CD8H”) or CD28 hinge region (“CD28H”), a CD28 transmembrane domain (“CD28tm” or “CD28TM”), a CD28 co-stimulatory domain (“CD28co”), no CD3z signaling domain (“noZ”/not shown), a P2A peptide, and a Flag tag. The membrane bound IL15-IL15Ra can further comprise an IgE signal protein (“IgE sp”), a linker such as G4S, and Myc. The top depicted schematic shows a construct with the following elements: IL3sp-IL3-G4S linker-CD33 scFv-Flag-CD28H-CD28tm-CD28co-P2A-IgE sp-IL15-G4S-Myc-IL15Ra (“nsIL3_CD33-dual CAR”). The bottom depicted schematic shows a construct with the following elements: CD8asp-CD33scFv-G4S linker-IL3-Flag-CD28H-CD28tm-CD28co-P2A-IgE sp-IL15-G4S linker-Myc-IL15Ra (“nsCD33_IL3-dual CAR”).

FIG. 29 is a schematic depicting transduction of a γδ T cell with a non-signaling CD33/CD123 dual-target CARs which co-express membrane-bound IL-15 (“ns-dCAR-mbIL15”). The figures show a viral vector comprising the ns-dCAR-mbIL15 used to transduce the γδ T cells. The transduced γδ T cell expresses the ns-dCAR with the anti-CD33 scFv and IL3 in the extracellular antigen recognition domain of the CAR and also expresses a membrane-bound IL15. Also shown are the γδ T-cell receptor (TCR), the NK cytotoxicity receptors (NCRs), the NKG2D receptor and DNAM-1 which bind to cognate ligands on the AML cell. The interaction between the ns-dCAR-mb15, including the binding of CD33 on the AML blast by the anti-CD33 scFv and the binding of CD123 by the IL3 as well as the binding of the NKG2D receptor to the NKG2D, results in specific killing of the AML cell.

FIG. 30A shows a construct of non-signaling CD33/IL3 dual target CAR that co-expresses membrane-bound IL15 (“nsIL3_CD33-Dual CAR” or “nsIL33_CD33-dCAR”). As shown in the figure, there is a detectable tag on the IL3, Flag and IL15.

FIGS. 30B and 30C shows flow cytometric analysis for CAR expression 6 days after transduction. Specifically, flow cytometric analysis of IL3/Flag expression (FIG. 30B) and IL15/Flag expression (FIG. 30C) was conducted in untransduced Jurkat cells and Jurkat cells transduced with ns33IL3_CD33-Dual at different MOIs.

FIGS. 31A and 31B show the time course dynamic for ns-dCAR-mb15 transduced Jurkat cell populations. Specifically, the figures show flow cytometric analysis of IL3/Flag expression at 2, 4 and 6 days after transduction. FIG. 31B shows that the population CAR-positive was about the same at Day 4 and Day 6 (40%) demonstrating stabilization of the population at about 4 days.

FIGS. 32-33C compare the cytotoxicity of nsCAR γδ T cell having different designs against AML cells. In these figures, “monoCAR” refers to ns33-sIL15 CAR γδ T cells. “dualCAR” refers to nsCD33-IL3-sIL15 or nsCD33-IL3-mb15 CAR γδ T cells (wherein “s15” is secreted IL15 and “mb15 is membrane-bound IL-15).

FIG. 32 shows flow cytometric analysis, 1 day after transduction, of side scatter fluorescence (SSC)/forward scatter fluorescence (FSC), SSC/γδ TCR (top two rows) and Flag/IL3 expression for γδ TCR positive T cells and γδ TCR negative T cells transduced with ns33-sIL15, ns33-IL3-sIL15 and ns33IL3-mb15. Higher transduction efficiency for sCAR transduced γδ T cells than the dualCAR γδ T cells was observed.

FIGS. 33A-33C are graphs of cytotoxicity (percentage) for different E:T ratios for untransduced γδ T cells, ns33-sIL15 CAR transduced γδ T cells (the monoCAR), ns33-IL3-sIL15 CAR transduced γδ T cells (a dual CAR), and ns33-IL3-mb15 transduced γδ T cells (a dual CAR) against HL-60, KG-1a and MOLM-13 cells, respectively. Cytotoxicity was determined by flow cytometric analysis of 7AAD/CFSE after co-culture of the γδ T cells with HL-60, KG-1a and MOLM-13 cells at E:T of 0, 0.25, 0.5. 1.0, 2.0, 4.0 and 8.0. Although the monoCAR demonstrated higher transduction than the dual CARs, the monoCAR and the dual CAR displayed similar cytotoxicity. This similar cytotoxicity of the monoCAR and the dual CARs may indicate that the dual CAR will show even higher killing when transduction is further improved. Comparison of the dual CARs shows that ns33-IL3-mb15 transduced γδ T cells (membrane-bound IL15) showed higher cytotoxicity than ns33-IL3-sIL15 CAR transduced γδ T cells (secreted IL15). ns33-IL3-mb15 transduced γδ T cells demonstrated the highest cytotoxicity overall, even with lower CAR transduction.

FIG. 34A-34C are graphs showing cytotoxicity (percentage) for different E:T ratios for untransduced gd T cells and ns33-IL3-mb15 transduced γδ T cells against HL-60, KG-1a and MOLM-13 cells, respectively. Ns33-IL3-mb15 showed enhanced killing in all three AML cell lines tested.

### DETAILED DESCRIPTION OF THE INVENTION

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

As used in the specification and the appended claims, the singular forms “a,” “an,” and “the” include plural referents unless the context clearly dictates otherwise. Thus, for example, reference to “a cell” includes a plurality of cells. In this specification and in the claims that follow, reference will be made to a number of terms that shall be defined to have the following meanings unless a contrary intention is apparent.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt. % to about 5 wt. %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include ±1%, 2%, 3%, +4%, +5%, +6%, 7%, 8±9%, or ±10%, or more of the numerical value(s) being modified. In addition, the phrase “about ‘x’ to ‘y’” includes “about ‘x’ to about ‘y’”. Numbers, ratios, concentrations, amounts, ranges and other numerical data should be construed as modified by the term “about” unless inconsistent with the context.

The term “drug resistant immunotherapy” or DRI is a strategy for treating cancer whereby anti-cancer immune cells, preferably Tb T-cells, are genetically engineered to resist the toxic effects of chemotherapy drugs which allows for the combined administration of chemotherapy and immunotherapy. Chemotherapy resistance or the acquisition of chemoresistance is a well-known phenomenon in the field of cancer treatment which results in chemotherapy-resistant tumor cells which are refractory to treatment. Such resistance to chemotherapeutic agents can arise from the expression of certain DNA, RNA or polypeptides that impact drug resistance genes, expression of a gene that conveys drug resistance, and/or the expression of a polypeptide that confers resistance to chemotherapeutic agents (collectively referred to herein as survival factors). The DRI strategy described herein uses chemoresistance to confer resistance to the modified immune cells that can be used in cancer immunotherapy. DRI γδ T-cells, for example, include Tb T-cells that have been genetically engineered to express a survival factor as described herein, including, but not limited to, a DNA, RNA or polypeptide that confers resistance to a chemotherapeutic agent. A polypeptide that confers resistance to a chemotherapeutic agent can be referred to herein as a “survival polypeptide”). DRI γδ T-cells that comprise the non-signaling CAR described herein can be referred to as “non-signaling CAR DRI γδ T-cells.”

The term “survival factor” refers to any agent now known or later discovered in the art that confers resistance to a chemotherapeutic agent, and/or to a chemotherapeutic agent treatment regimen and/or allows the cells comprising the survival factor to survive in a treatment environment (such as a chemotherapy treatment environment). The phrase “confers resistance” and the like encompasses the acquisition of resistance to a chemotherapeutic agent or improvement in resistance to a chemotherapeutic agent. The “survival factor” includes an agent that confers resistance to a chemotherapeutic agent when it is expressed by the γδ T cell. The “survival factor” can thus be a DNA, RNA or polypeptide that is expressed by the γδ T cells (e.g., encoded by a drug resistance gene) and that confers resistance to a chemotherapeutic agent. As described herein, the γδ T cell can be modified to express the DNA, RNA or polypeptide that confers resistance to a chemotherapeutic drug by including a vector which, for example, expresses a gene, a gene fragment, a DNA, an siRNA, or an mRNA, that encodes the survival factor that confers resistance to a chemotherapeutic agent. In yet other aspects, the survival factor is a DNA that confers resistance to a chemotherapeutic agent. In further aspects, the survival factor is an RNA (e.g., a RNAi, siRNA, micoRNA, or mRNA) that confers resistance to a chemotherapeutic agent.

In certain aspects, the survival factor is a polypeptide that confers resistance to a chemotherapeutic agent; for example, the polypeptide confers resistance when it is expressed by the γδ T cells. In certain embodiments, the survival factor is an alkylguanine transferase (AGT; or alkylguanine-DNA-alkyltransferase). In certain specific aspects, the survival factor is MGMT (including the P140K mutant of human O(6)-methylguanine-DNA-methyltransferase), a multidrug resistance protein (such as MDR1), or 5′ nucleotidase II (NT5C2). Other survival factors include, for example, a drug resistant variant of dihydrofolate reductase (L22Y-DHFR) and thymidylate synthase. Multidrug resistance proteins (MDPs) have been described, for example, in Sodani et al. (2012), Chin J Cancer 31(2): 58-72 and Zhang et al. (2015), The AAPS Journal 17: 802-812, the contents of each of which are expressly incorporated by reference herein. MDPs that can be used as a survival factor include, for example, MDR1, MDR2, MDR3, MDR4, MDR5, MDR6, MDR7, MDR8 and MDR9. Cancer multi-drug resistance genes have also been described, for example, in Lau et al. 2020, Systematic functional identification of cancer multi-drug resistance genes. Genome Biol 21, 27; the contents of which are expressly incorporated by reference herein. In addition, numerous genes associated with resistance to platinum drugs (e.g., cisplatin, carboplatin and oxaliplatin) were described in Huang et al. (2021), A highly annotated database of genes associated with platinum resistance in cancer. Oncogene 40, 6395-6405; the contents of which are expressly incorporated by reference herein. The survival factor can be a polypeptide that confers resistance to a chemotherapeutic agent, including, but not limited, the specific chemotherapeutic agents described herein. In certain specific aspects, the survival factor in is MGMT. In yet additional aspects, the survival factor is MDR1. Other polypeptides that confer resistance may be used or expressed by the cell depending on the nature of the treatment environment (i.e., what other treatment regimens are being given to the patient in combination with the cells compositions of the present disclosure). MGMT repairs alkylating lesions of the DNA by removing mutagenic adducts from the 06 position of guanine. Such mutagenic adducts can be caused by alkylating agents (including, but not limited to, temozolomide). Thus, MGMT is a polypeptide that confers resistance to alkylating agents such as temozolomide and cyclophosphamide.

An additional example of a survival factor is a mutated form of BCL2, for example, the G101V mutant that mediates resistance to venetoclax, a BCL2 inhibitor. Bernareggi et al., for example, described CRISPR-Cas9 technology to knock-in the BCL2 G101V mutation (BCL2G101V) in iPSCs which were differentiated into NK cells (Bernareggi et al. (2022), Blood 140(1): 7407-7408).

Additional examples of survival factors include mutant or modified form of dihydrofolate reductase (DHFR) in addition to the L22Y-DHFR described above. Various mutant forms of DHFR have been described that have increased resistance to inhibition by antifolate agents such as methotrexate. In a specific embodiment, the drug resistance gene of the present invention may be a nucleotide sequence encoding a mutant form of human wild-type DHFR (GenBank: AAN71996.1) that contains at least one mutation for resistance to treatment with an anti-folate drug such as methotrexate. In a specific embodiment, the DHFR mutant contains at least one mutated amino acid at position G15, L22, F31 or F34, preferably at positions L22 or F31 (Schweitzer, Dicker et al., 1990); WO 94/24277; U.S. Pat. No. 6,642,043). In a specific embodiment, the mutant DHFR contains two mutated amino acids at positions L22 and F31. In certain embodiments, the serine residue at position 15 is replaced with a tryptophan residue (referring to the amino acid positions in the wild-type DHFR polypeptide registered in GenBank: AAH71996.1). In another specific embodiment, the leucine residue at position 22 is replaced with an amino acid that can disrupt the binding of the mutant DHFR to antifolate agents, for example, uncharged amino acid residues such as phenylalanine or tyrosine. In another specific embodiment, the phenylalanine residue at positions 31 or 34 is replaced with a low molecular weight hydrophilic amino acid residue such as alanine, serine or glycine.

An additional example of a survival factor is a mutant or modified form of inosine 5′-monophosphate dehydrogenase II (IMPDH2), an enzyme that limits the rate of de novo synthesis of guanosine nucleotides. A mutant or modified form of IMPDH2 is a gene that confers resistance to an IMPDH inhibitor. Inhibitors of IMPDH can be mycophenolic acid (MPA) or its prodrug mycophenolate mofetil (MMF). The IMPDH2 mutant can contain at least one, or sometimes two, mutations in the MAP binding site of human wild-type IMPDH2 (NP_000875.2) that result in a significant increase in resistance to the IMPDH inhibitor. Mutations that affect the T333 and/or S351 positions have been described. In a specific embodiment, the threonine residue at position 333 is replaced with an isoleucine residue and the serine residue at position 351 is replaced with a tyrosine residue. Amino acid positions correspond to GenBank NP_000875.2).

Yet another exemplary survival factor is a mutant form of calcineurin. The survival factor can be a nucleotide sequence that encodes a mutant form of calcineurin resistant to a calcineurin inhibitor such as FK506 and/or CsA. In a specific embodiment, the mutant form can contain at least one mutant amino acid in the wild-type calcineurin heterodimer at positions V314, Y341, M347, T351, W352, L354, K360, preferably double mutations at positions T351 and L354 or V314 and Y341. In certain embodiments, the valine residue at position 341 can be replaced with a lysine or arginine residue, the tyrosine residue at position 341 can be replaced with a phenylalanine residue; methionine at position 347 can be substituted for the residue of glutamic acid, arginine or tryptophan; the threonine at position 351 can be replaced by a glutamic acid residue; the tryptophan residue at position 352 can be replaced with a cysteine, glutamic acid or alanine residue, the serine at position 353 can be replaced with a histidine or asparagine residue, leucine at position 354 can be replaced with an alanine residue; the lysine at position 360 can be substituted for an alanine or phenylalanine residue in the sequence corresponding to the sequence recorded in GenBank: ACX34092.1. In additional embodiments, the mutant form may contain at least one mutant amino acid in the wild-type calcineurin heterodimer at positions V120, N123, L124 or K125, preferably double mutations at positions L124 and K125. In a specific embodiment, the valine at position 120 may be substituted for a serine, aspartic acid, phenylalanine, or leucine residue; asparagine at position 123 can be substituted for tryptophan, lysine, phenylalanine, arginine, histidine, or serine; the leucine at position 124 can be substituted for a threonine residue; lysine at position 125 can be replaced by alanine, glutamic acid, tryptophan, or two residues such as leucine-arginine or isoleucine-glutamic acid can be added after lysine at position 125 in the amino acid sequence corresponding to the sequence recorded in GenBank: ACX34095.1

An additional example of a survival factor is a specific mutant of the human topoisomerase gene. For example, mutations at the Arg486 and Glu571 mutations in the human topoisomerase II gene, confer amsacrine resistance (S. Patel, B. A. Keller and LM Fisher. Molecular Pharmacology Vol. 57, 2000, pp. 784-791).

In additional aspects, the survival factor is a DNA or an RNA that confers resistance to a chemotherapeutic agent.

In yet further embodiments, the gd T cell expresses at least two survival factors.

The co-expressed cytokine IL15, IL2 and/or IL7 can be membrane-tethered and/or secreted. In some embodiments, the IL15, IL7 and/or IL2 is membrane-tethered. In yet additional aspects, the IL15, IL7 and/or IL2 is secreted. In certain aspects, the IL15, IL2 and/or IL7 is recombinant. Specific membrane-bound IL15 have been described for example, in Hurton et al. (2016), PNAS 113(48): E7788-E7797 and Hurton, Lenka, “TETHERED IL-15 TO AUGMENT THE THERAPEUTIC POTENTIAL OF T CELLS EXPRESSING CHIMERIC ANTIGEN RECEPTOR: MAINTAINING MEMORY POTENTIAL, PERSISTENCE, AND ANTITUMOR ACTIVITY” (2014). The University of Texas MD Anderson Cancer Center UTHealth Graduate School of Biomedical Sciences Dissertations and Theses (Open Access). 421. digitalcommons.library.tmc.edu/utgsbs_dissertations/421; the contents of which are expressly incorporated by reference herein.

By “administration” is meant introducing a compound, biological materials including a cell population, or a combination thereof, or a composition comprising any of the aforementioned compounds, biological materials (e.g., a cell population), or a combination thereof, of the present invention into a human or animal subject. One preferred route of administration of the compounds is intravenous. Another preferred route is parenteral. “Parenteral” refers to a route of administration that is associated with injection, including intraorbital, infusion, intraarterial, intracapsular, intracardiac, intradermal, intramuscular, intraperitoneal, intrapulmonary, intraspinal, intrasternal, intrathecal, intracranial, intrauterine, intravenous, subarachnoid, subcapsular, subcutaneous, transmucosal, or transtracheal. Other exemplary routes of administration of the compounds may be intraperitoneal or intrapleural, or via a catheter to the brain. However, any route of administration, such as oral, topical, subcutaneous, peritoneal, intra-arterial, inhalation, vaginal, rectal, nasal, introduction into the cerebrospinal fluid, intracranial, or instillation into body compartments can be used. Direct injection into a target tissue site such as a solid tumor is also contemplated. For example, intracranial administration of the γδ T-cells for the treatment of a glioma or other intracranial tumor can be used.

The term “cancer”, as used herein, shall be given its ordinary meaning, as a general term for diseases in which abnormal cells divide without control. Cancer cells can invade nearby tissues and can spread through the bloodstream and lymphatic system to other parts of the body. When normal cells lose their ability to behave as a specified, controlled and coordinated unit, a tumor is formed. Generally, a solid tumor is an abnormal mass of tissue that usually does not contain cysts or liquid areas (some brain tumors do have cysts and central necrotic areas filled with liquid). A single tumor may even have different populations of cells within it, with differing processes that have gone awry. Solid tumors may be benign (not cancerous), or malignant (cancerous). Different types of solid tumors are named for the type of cells that form them. Examples of solid tumors are sarcomas, carcinomas, and lymphomas. Leukemias (cancers of the blood) generally do not form solid tumors. Carcinoma is cancer that begins in the skin or in tissues that line or cover internal organs. Glioma is a tumor that arises from the supportive (“gluey”) tissue of the brain, called glia, which helps to keep the neurons in place and functioning well. Sarcoma is cancer that begins in bone, cartilage, fat, muscle, blood vessels, or other connective or supportive tissue. Leukemia is cancer that starts in blood-forming tissue such as the bone marrow, and causes large numbers of abnormal blood cells to be produced and enter the bloodstream. Lymphoma is cancer that begins in the cells of the immune system.

Representative cancers include, but are not limited to, Acute Lymphoblastic Leukemia (ALL), Adult; Acute Lymphoblastic Leukemia, Childhood; Acute Myeloid Leukemia (AML), Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Glioblastoma, Childhood; Glioblastoma, Adult; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood: Carcinoid Tumor, Childhood; Carcinoid Tumor, Gastrointestinal; Carcinoma, Adrenocortical; Carcinoma, Islet-cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chrome Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ. Cell Tumor, Childhood; Extragonadal Germ. Cell Tumor; Extrahepatic Bile Duct Cancer; Eye Cancer, Intraocular Melanoma; Eye Cancer, Retinoblastoma; Gallbladder Cancer; Gastric (Stomach) Cancer; Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma. Childhood Brain Stem; Glioma. Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hvpopharyngeal Cancer; Hypothalamic and Visual Pathway Glioma, Childhood; Intraocular Melanoma; Islet-cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chrome Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's; Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplasia Syndromes; Myelogenous Leukemia, Chrome; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Neurofibroma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood’, Pancreatic Cancer, Islet-cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland' Cancer, Childhood; Sarcoma, Ewing's Family of Tumors; Sarcoma, Kaposi's; Sarcoma (Osteosarcoma)/Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor, among others.

A tumor can be classified as malignant or benign. In both cases, there is an abnormal aggregation and proliferation of cells. In the case of a malignant tumor, these cells behave more aggressively, acquiring properties of increased invasiveness. Ultimately, the tumor cells may even gain the ability to break away from the microscopic environment in which they originated, spread to another area of the body (with a different environment, not normally conducive to their growth), and continue their rapid growth and division in this new location. This is called metastasis. Once malignant cells have metastasized, achieving a cure or treatment is more difficult. Benign tumors have less of a tendency to invade and are less likely to metastasize.

The term “fusion protein”, as used herein, refers to a chimeric molecule, which comprise, for example, an antigen recognition domain, and at least one heterologous portion, i.e., a portion with which it is not naturally linked in nature. The amino acid sequences may normally exist in separate proteins that are brought together in the fusion polypeptide or they may normally exist in the same protein but are placed in a new arrangement in the fusion polypeptide. Fusion proteins may be created, for example, by chemical synthesis, or by creating and translating a polynucleotide in which the peptide regions are encoded in the desired relationship.

The methods of treatment described herein comprising administration of the non-signaling CAR γδ T-cells can be used to reduce a cancer or tumor. The terms “reducing a cancer,” “inhibition of cancer,” “inhibiting cancer,” “preventing cancer recurrence,” and similar terms and are used interchangeably herein and refer to one or more of a reduction in the size or volume of a tumor mass, a decrease in the number of metastasized tumors in a subject, a decrease in the proliferative status (the degree to which the cancer cells are multiplying) of the cancer cells, prevention of recurrences of previous tumors or the development of new metastases, prevention of relapse, prevention of post-transplant relapse, achieving remission, and the like. In certain aspects, measurable residual disease (MRD) can be assessed during and/or after the treatment regimen. For example, MRD assessment in AML can include multiparameter flow cytometry-based MRD (MFC-MRD) and/or molecular MRD (Mol-MRD) assessed by qPCR (Dohner et al. (2022), Diagnosis and management of AML in adults: 2022 recommendations from an international expert panel on behalf of the ELN, Blood 140(12): 1345-1377; the contents of which are expressly incorporated by reference herein).

The methods of treatment described herein comprise administration of an effective amount of the non-signaling CAR γδ T-cells and optionally, an effective amount of a chemotherapeutic agent can be used to reduce a tumor or reduce cancer. The term “reducing a tumor” as used herein refers to a reduction in the size or volume of a tumor mass, a decrease in the number of metastasized tumors in a subject, a decrease in the proliferative status (the degree to which the cancer cells are multiplying) of the cancer cells, and the like.

The method of treatment described herein can comprise administration of the non-signaling CAR γδ T-cells and optionally, a chemotherapeutic agent and can be used to treat a hematologic cancer, for example, leukemia or lymphoma. The lymphoma or leukemia can be selected from the group consisting of acute lymphoblastic leukemia (ALL), acute myeloid leukemia (AML), peripheral T-cell lymphoma, not otherwise specified (PTCL-NOS); angioimmunoblastic T-cell lymphoma (AITL), anaplastic large cell lymphoma (ALCL), enteropathy-associated T-cell lymphoma (EATL), hepatosplenic T-cell lymphoma (HSTL), extranodal NK/T-cell lymphoma nasal type, cutaneous T-cell lymphoma, primary cutaneous ALCL, T cell prolymphocytic leukemia and T-cell acute lymphoblastic leukemia, myelodysplastic syndromes, myelodysplastic/myeloproliferative neoplasms, multiple myeloma, chronic myelogenous leukemia, chronic myeloproliferative neoplasms, Burkitt lymphoma, chronic lymphocytic leukemia, Hodgkin lymphoma, and hairy cell leukemia.

The term “chemotherapeutic agent” as used herein refers to a compound or a derivative thereof that can interact with a cancer cell, thereby reducing the proliferative status of the cell and/or killing the cell for example, by impairing cell division or DNA synthesis, or by damaging DNA, effectively targeting fast dividing cells. Examples of chemotherapeutic agents include, but are not limited to, alkylating agents (e.g., cyclophosphamide, ifosfamide, temozolomide, chlorambucil, bendamustine); metabolic antagonists (e.g., methotrexate (MTX), trimetrexate (TMTX), pralatrexate, 5-fluorouracil or derivatives thereof); a substituted nucleotide; a substituted nucleoside; DNA demethylating agents (also known as antimetabolites; e.g., azacitidine); antitumor antibiotics (e.g., mitomycin, adriamycin); plant-derived antitumor agents (e.g., vincristine, vindesine, vinblastine, TAXOL®, paclitaxel, abraxane); cisplatin; carboplatin; oxaliplatin; etoposide; and the like. Such agents may further include, but are not limited to, the anti-cancer agents trimetrexate (TMTX); temozolomide (TMZ); anthracycline; raltitrexed; S-(4-Nitrobenzyl)-6-thioinosine (NBMPR); 6-benzyguanidine (6-BG); a nitrosourea (rabinopyranosyl-N-methyl-N-nitrosourea (Aranose), Carboplatin, Carmustine (BCNU, BiCNU), Chlorozotocin, Bendamustine, Bleomycin; Cyclophosphamide, Chlorambucil, Cisplatin, Cladribine, Cytarabine, Doxorubicin, Etoposide, Ethylnitrosourea (ENU), Fludarabine, Fotemustine, Gemcitabine, Ifosfamide, Lomustine (CCNU), Methotrexate, Mitoxantrone, Nimustine, N-Nitroso-N-methylurea (NMU), Oxaliplatin, Pentostatin, Pralatrexate, Ranimustine (MCNU), Semustine, Streptozocin (Streptozotocin)); Vincristine; cytarabine; and camptothecin; or a therapeutic derivative of any thereof; FMS-like tyrosinase kinase 3 (FLT3) inhibitors; isocitrate dehydrogenase 1 or 2 (IDH1/2) inhibitors; target effector molecules of apoptosis, metabolism, epigenetics, stemness, and immune escape including, for example, B-cell lymphoma 2 (BCL2) inhibitors, hypomethylating agents (HM), smoothened (SMO) inhibitors, and CD33 monoclonal antibody. For example, chemotherapeutic agent used to treat AML can include cytarabine and/or anthracycline.

In certain aspects, the invention is a method of treating AML in a patient in need thereof comprising administering to said patient a composition comprising an effective amount of the non-signaling CAR γδ T-cells described herein. In one example, the non-signaling CAR has an antigen binding domain that binds CD19. In another example, the non-signaling CAR has an antigen binding domain that binds CD33. The non-signaling CAR can be a monoCAR that binds CD33 or can be a dualCAR (e.g, a tandem dualCAR) that binds CD33 and second tumor antigen, for example a dualCAR that binds CD33 and CD123. The non-signaling CAR γδ T-cells can comprise a co-stimulatory domain, for example, only one co-stimulatory domain. Also, the non-signaling CAR γδ T-cells can further express a cytokine selected from the group consisting of IL15, IL7 and/or IL2, and the method can optionally further comprise co-administering to said subject the chemotherapeutic agent in an amount sufficient to increase stress antigen expression on the cancer or tumor cells. In addition, the non-signaling CAR γδ T-cells can further comprise a survival factor that confers resistance to a chemotherapeutic agent, and the method can further comprise co-administering to said subject the chemotherapeutic agent in an amount sufficient to increase stress antigen expression on the cancer or tumor cells. The chemotherapeutic agent can, for example, be cytarabine and/or an anthracycline such as daunorubicin or idarubicin. An exemplary survival polypeptide that confers resistance to cytarabine and/or the anthracycline is MDR1.

In certain additional aspects, the invention is a method of treating ALL or a B-cell derived lymphoma in a patient in need thereof comprising administering to said patient a composition comprising an effective amount of the non-signaling CAR γδ T-cells described herein. In one example, the non-signaling CAR has an antigen binding domain that binds CD19. The non-signaling CAR can be a monoCAR that binds CD19 or can be a dualCAR (e.g., a tandem dualCAR) that binds CD19 and second tumor antigen, for example, a dual CAR (e.g., a tandem dualCAR) that binds CD19 and CD22, or a dualCAR (e.g., a tandem dualCAR) that binds CD19 and CD20. The non-signaling CAR γδ T-cells can comprise a co-stimulatory domain, for example, only one co-stimulatory domain. Also, the non-signaling CAR γδ T-cells can further express a cytokine selected from the group consisting of IL15, IL7 and/or IL2, and the method can optionally further comprise co-administering to said subject the chemotherapeutic agent in an amount sufficient to increase stress antigen expression on the cancer or tumor cells. In addition, the non-signaling CAR γδ T-cells can further comprise a survival factor that confers resistance to a chemotherapeutic agent, and the method can further comprise co-administering to said subject the chemotherapeutic agent in an amount sufficient to increase stress antigen expression on the cancer or tumor cells. The chemotherapeutic agent can, for example, be vincristine or anthracycline drug such as doxorubicin or daunorubicin. An exemplary survival polypeptide that confers resistance to vincristine and/or the anthracycline is MDR1.

In further aspects, the invention is a method of treating chronic lymphocytic leukemia (CLL) in a patient in need thereof comprising administering to said patient a composition comprising an effective amount of the non-signaling CAR γδ T-cells described herein. In one example, the non-signaling CAR has an antigen binding domain that binds CD19. In another example, the non-signaling CAR has an antigen binding domain that binds CD33. In specific aspects, the non-signaling CAR can be a monoCAR that binds CD19 or can be a dualCAR (e.g., a tandem dualCAR) that binds CD19 and second tumor antigen, for example, a dual CAR (e.g., a tandem dualCAR) that binds CD19 and CD22, or a dualCAR (e.g., a tandem dualCAR) that binds CD19 and CD20. The non-signaling CAR γδ T-cells can comprise a co-stimulatory domain, for example, only one co-stimulatory domain. Also, the non-signaling CAR γδ T-cells can further express a cytokine selected from the group consisting of IL15, IL7 and/or IL2, and the method can optionally further comprise co-administering to said subject the chemotherapeutic agent in an amount sufficient to increase stress antigen expression on the cancer or tumor cells. In addition, the non-signaling CAR γδ T-cells can further comprise a survival factor that confers resistance to a chemotherapeutic agent, and the method can further comprise co-administering to said subject the chemotherapeutic agent in an amount sufficient to increase stress antigen expression on the cancer or tumor cells. The chemotherapeutic agent can, for example, be cyclophosphamide, fludarabine, and/or rituximab. An exemplary survival polypeptide that confers resistance to cyclophosphamide is MGMT. An exemplary polypeptide that confer resistance to fludarabine is aminoglycoside-3′-phosphotransferase-IIa (APH(3′)-IIa).

In further aspects, the invention is a method of treating lymphoma (such as Non-Hodgkins B-cell Lymphoma or diffuse large B-cell lymphoma) in a patient in need thereof comprising administering to said patient a composition comprising an effective amount of the non-signaling CAR γδ T-cells described herein. In one example, the non-signaling CAR has an antigen binding domain that binds CD19. In another example, the non-signaling CAR has an antigen binding domain that binds CD33. The non-signaling CAR can be a monoCAR that binds CD19 or can be a dualCAR (e.g., a tandem dualCAR) that binds CD19 and second tumor antigen, for example, a dual CAR (e.g., a tandem dualCAR) that binds CD19 and CD22, or a dualCAR (e.g., a tandem dualCAR) that binds CD19 and CD20. For example, the non-signaling CAR γδ T-cells can comprise a co-stimulatory domain, for example, only one co-stimulatory domain. Also, the non-signaling CAR γδ T-cells can further express a cytokine selected from the group consisting of IL15, IL7 and/or IL2, and the method can optionally further comprising, co-administering to said subject the chemotherapeutic agent in an amount sufficient to increase stress antigen expression on the cancer or tumor cells. In addition, the non-signaling CAR γδ T-cells can further comprise a survival factor that confers resistance to a chemotherapeutic agent, and the method can further comprise co-administering to said subject the chemotherapeutic agent in an amount sufficient to increase stress antigen expression on the cancer or tumor cells. The chemotherapeutic agent can, for example, be cyclophosphamide, fludarabine, and/or rituximab.

The term “chimeric antigen receptor(s) (CAR(s)),” as used herein, refers to artificial T-cell receptors, T-bodies, single-chain immunoreceptors, chimeric T-cell receptors, or chimeric immunoreceptors, for example, and encompass engineered receptors that graft an artificial specificity (for example, an antigen recognition domain) onto a particular immune effector cell, for example, γδ T-cells. Conventional signaling CARs generally comprise an intracellular activation domain (e.g. a CD3z signaling domain), a transmembrane domain, and an extracellular domain that may vary in length and that comprises an antigen recognition domain. The non-signaling CARs as described herein lack an intracellular T-cell activation domain, such as that of CD3z. The terms “non-signaling CAR,” “ns-CAR,” “nsCAR,” “NS-CAR,” “NSCAR,” and the like are used interchangeably herein. A non-signaling CD19 CAR (a non-signaling CAR with an extracellular antigen recognition domain that binds CD19) can be referred to herein as a “non-signaling CD19 CAR,” “NS-CD19 CAR,” “ns-CD19 CAR,” “non-signaling anti-CD19 CAR,” and the like. Similarly, a non-signaling CD33 CAR (a non-signaling CAR with an extracellular antigen recognition domain that binds CD33) can be referred to herein as a “non-signaling CD33 CAR,” “NS-CD33 CAR,” “ns-CD33 CAR,” “non-signaling anti-CD33 CAR,” and the like. As described herein, the non-signaling CAR can, for example, be a monoCAR or a dualCAR which includes tandem dualCARs and separate dualCARs.

Domains of the CAR can be linked directed, or indirectly linked by a peptide or peptides (which peptides can be referred to herein as “linker peptide” or “linker peptides”). The linker peptides can be the same or different.

The specificity of CAR designs may be derived from ligands of receptors (e.g., peptides). In certain cases, the spacing of the antigen-recognition domain can be modified to reduce activation-induced cell death (AICD). In certain cases, the endodomain of the CARs comprise domains for additional co-stimulatory signaling, such as, but not limited to, FcR, CD27, CD28, CD137 (4-1BB), DAP 10, and/or OX40. In some cases, molecules can be co-expressed with the CAR, including co-stimulatory molecules, reporter genes for imaging (e.g., for positron emission tomography), gene products that allow cells expressing the CAR to survive in a treatment environment created by an additional therapeutic treatment, gene products that conditionally ablate the cells expressing the CAR upon addition of a pro-drug, homing receptors, chemokines, chemokine receptors, cytokines, and cytokine receptors.

The terms “antigen recognition domain,” “antigen recognition moiety,” “antigen binding domain,” “antigen binding moiety,” and the like, are used interchangeably herein. Similarly, the terms, “transmembrane domain,” “transmembrane moiety,” “transmembrane region,” and the like are used interchangeably; the terms “hinge domain,” “hinge moiety,” and “hinge region,” and the like are used interchangeably; the terms “intracellular signaling domain,” “signaling domain,” “signaling moiety,” “signaling region,” and the like are used interchangeably herein.

The endodomain of a signaling CAR generally includes a T-cell activation domain, usually derived from CD3z chain of the T-cell receptor, as well as co-stimulatory domains, for example the immunoreceptor tyrosine-based activation motif containing regions of CD28 and 4-1BB. The non-signaling CARs described herein lack the T-cell activation domain (such as the CD3z T-cell activation domain) but can include one or more co-stimulatory domains. In additional aspects, the non-signaling CAR lacks the T-cell activation domain and also does not include a co-stimulatory domain.

The phrase “therapeutically effective amount” or an “effective amount” in the context of the administration of an agent or composition to a subject, refers to an amount capable of having any detectable, positive effect on any symptom, aspect, or characteristic of a disease, disorder or condition, when administered to the subject; the agent or composition can be administered either alone or as part of a pharmaceutical composition and either in a single dose or as part of a series of doses. The therapeutically effective amount or effective amount can be ascertained by measuring relevant physiological effects, and it can be adjusted in connection with the dosing regimen and diagnostic analysis of the subject's condition, and the like. In reference to cancer or pathologies related to unregulated cell division, a therapeutically effective amount or an effective amount can refer to that amount which has the effect of (1) reducing the size of a tumor (i.e. tumor regression), (2) inhibiting (that is, slowing to some extent, preferably stopping) aberrant-cell division, for example cancer cell division, (3) preventing or reducing the metastasis of cancer cells, (4) relieving to some extent (or, preferably, eliminating) one or more symptoms associated with a pathology related to or caused in part by unregulated or aberrant-cellular division, including for example, cancer, (5) increasing the survival or life expectancy of the subject, and/or (6) decreasing the risk of relapse. An “effective amount” can also be an amount that results in desirable PD and PK profiles and/or desirable immune cell profiling or reconstitution upon administration of the therapeutically active compositions of the invention. An “effective amount” can also be an amount that achieves a recited effect or result; for example, an effective amount of a chemotherapeutic agent that, alone or when in combination with another agent, can be an amount that reduces the size of a tumor and/or increases stress antigen expression on the tumor cells, and/or has a cytotoxic effect.

The terms “treating” or “treatment” of a disease (or a condition or a disorder) as used herein refer to inhibiting the disease (slowing or arresting its development), providing relief from the symptoms or side-effects of the disease (including palliative treatment), preventing or delaying recurrence, and causing regression of the disease. With regard to cancer, these terms also mean that the life expectancy of an individual affected with a cancer may be increased or that one or more of the symptoms of the disease will be reduced. With regard to cancer, “treating” also includes enhancing or prolonging an anti-tumor or anti-cancer response in a subject.

As used herein any form of administration of a “combination”, “combined therapy” and/or “combined treatment regimen,” or “co-administration” or “co-administering,” or the like, refers to administration of at least two therapeutically active drugs or compositions (e.g., administration of the γδ T-cells and chemotherapeutic agent, or pharmaceutical compositions thereof), simultaneously or substantially simultaneously in either separate or combined formulations, or sequentially at different times separated by minutes, hours, days, weeks, or months, but in some way act together to provide the desired therapeutic response, for example, as part of the same treatment regimen.

The terms “enhance,” “enhancing” “enhanced”, or the like, as used herein, refers to an increase in the response or outcome referred to. For example, “enhancing cytotoxicity” refers to increasing cytotoxicity. Similarly, enhanced persistence means increasing persistence. The term “enhancing” and the like can also encompass allowing a subject or tumor cell to improve its ability to respond to a treatment disclosed herein. An enhanced response can comprise an increase in responsiveness (cytotoxicity and/or persistence) of at least about 5%, 10%, 15%, 20%, 25%, 30%, 35%, 40%, 45%, 50%, 55%, 60%, 65%, 70%, 75%, 80%, 85%, 90%, 95% or 98% or more. The enhanced responsiveness can encompass enhanced cytotoxicity to the cancer or tumor and/or enhanced persistence.

The terms “reduce,” “reducing” “reduced”, or the like, as used herein, refers to a decrease in the response or outcome referred to. For example, “reduced cytotoxicity” to a normal, non-cancerous cell refers to a decreased cytotoxicity to that normal, non-cancerous cell, for example, as compared with the cytotoxicity to a cancer cell. A reduced cytotoxicity can comprise a decrease in cytotoxicity of at least about 5%, 10%, 15%, 20%, 25%, 30%, 35%, 40%, 45%, 50%, 55%, 60%, 65%, 70%, 75%, 80%, 85%, 90%, 95% or 98% or more.

The terms “subject” and “patient” as used herein include humans, mammals (e.g., cats, dogs, horses, etc.), living cells, and other living organisms. A living organism can be a mammal. Typical patients are mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like. Preferably, a system includes a sample and a subject. The term “living host” refers to host or organisms noted above that are alive and are not dead. The term “living host” refers to the entire host or organism and not just a part excised (e.g., a liver or other organ) from the living host. In preferred aspects, the subject or patient is a human subject or patient.

The term “γδ T-cells, “gd T-cells,” and “gamma delta T-cells” as used herein refers to a subset of T-cells that express a distinct T-cell receptor (TCR) on their surface. The majority of T-cells have a TCR composed of two glycoprotein chains called a- and p-TCR chains. In contrast, in γδ T-cells, the TCR is made up of one 7-chain and one 6-chain. This group of T-cells is usually much less common than αβ T-cells. γδ T-cells are unique amongst T-cell types in that they do not require antigen processing and MHC presentation of peptide epitopes. Furthermore, γδ T-cells are believed to have a prominent role in recognition of lipid antigens, and to respond to stress-related antigens such as MIC-A and MIC-B and other ligands of the NKG2D receptor. In humans, Vδ1 and Vδ2 γδ T-cells are the two main populations of γδ T-cells as based on their TCR expression. Vδ2 γδ T-cells are circulating lymphocytes and constitute the majority of peripheral blood γδ T-cells. Meanwhile, Vδ1 γδ T-cells are generally resident lymphocytes, abundant in mucosal surfaces and epithelia of the digestive, respiratory and urogenital tracts (Caron et al. (2021), Front Immunol. https://doi.org/10.3389/fimmu.2021.666983). γδ T cells include Vγ9Vδ2 T-cells and Vγ9Vδ1 T-cells.

Human γδ T-cells can also exhibit an antigen-presenting capacity. Similar to dendritic cells (DCs), blood Vγ9Vδ2 T-cells are able to respond to signals from microbes and tumors and prime CD4⁺ and CD8⁺ T-cells. TS T-APCs are believed to cross-present antigens directly to CD8⁺ T-cells. The intracellular protein degradation and endosomal acidification are significantly delayed in γδ T-cells in comparison to monocyte-derived DCs. The antigens are transported across IRAP (Insulin-Regulated Amino Peptidase)-positive early and late endosomes, and their processing consists of an export to the cytosol for degradation by the proteasome before being imported into an MHC-I-loading compartment. Activated γδ T-cells are able to phagocytose tumor antigens and apoptotic or live cancer cells possibly through the scavenger receptor CD36 in a C/EBPα (CCAAT/enhancer-binding protein a)-dependent mechanism and mount a tumor antigen-specific CD8+ T-cell response. γδ T-cells can also induce DC maturation through TNF-α production. Overall, γδ T-cells can process a wide range of antigens for presentation and stimulate other immune cells. Therefore, γδ T-cells' role in response to infections or cancer may be leveraged to design new strategies in order to improve clinical response of human γδ T-cell-based immunotherapy. Increased tumor immunogenicity (e.g., increased upregulation of ligands for the NKG2D receptor), e.g., resulting from a chemotherapeutic agent or DDR inhibition is uniquely conducive to γδ T-cell-mediated tumor immunosurveillance, and ultimately tumor cell killing by γδ T-cells.

A cell composition or population of cells can be enriched for the γδ T-cells or the engineered γδ T-cells, for example. The term “enriched”, as used herein, refers to increasing the total percentage of one or more cytotoxic immune cell types present (e.g., γδ T-cells and/or NK cells) in a sample, relative to the total percentage of the same one or more cell types prior to enrichment, as disclosed herein. For example, a sample that is “enriched” for one or more types of cytotoxic immune cell may comprise between about 10% to 100% of the one or more cytotoxic immune cell types in the sample, whereas the total percentage of one or more of the cytotoxic immune cell types in a sample prior to enrichment was, for example, between 0% and 10%. Preferably, an enriched sample comprises at least 10%, 15%, 20%, 25%, 30%, 35%, 40%, 50%, 60%, 70%, 80%, 90% or 100%, of one or more types of cytotoxic immune cell. Samples may be enriched for one or more cell types using standard techniques, for example, flow cytometry techniques. The term “highly enriched”, as used herein, refers to increasing the total percentage of one or more cytotoxic immune cell types in a sample such that the one or more cytotoxic immune cell types may comprise between at least about 70% to about 100% of the cytotoxic immune cell type in the sample, whereas the total percentage of that same type of cytotoxic immune cell prior to enrichment was, for example, between 0% and 10%. Preferably, a highly enriched sample comprises at least 60%, 65%, 70%, 75%, 80%, 85%, 90%, 95%, 99% or more of one or more types of cytotoxic immune cell. Samples may be highly enriched for one or more cell types using standard techniques, for example, flow cytometry techniques.

A cell composition or population can comprise an expanded population of γδ T-cells or the engineered γδ T-cells, or any subset thereof for example. The terms “expanded” and “expansion” as used herein with regard to expansion of one or more cytotoxic immune cells in a sample means to increase in the number of one or more cytotoxic immune cells in a sample by, for example about at least 2-fold, preferably by about 5-fold, preferably by at least 10-fold, preferably about at least 50-fold or more. Expansion of a cytotoxic immune cell population can be accomplished by any number of methods as are known in the art. For example, T-cells can be rapidly expanded using non-specific T-cells receptor stimulation (optionally, in the presence of feeder lymphocytes) and either interleukin-2 (IL-2) or interleukin-15 (IL-15), with IL-2 being preferred. The non-specific T-cell receptor stimulus can, for example, include around 30 ng/ml of OKT3, a mouse monoclonal anti-CD3 antibody (available from ORTHO-MCNEIL®, Raritan, N.J.). Alternatively T-cells can be rapidly expanded by stimulation of peripheral blood mononuclear cells (PBMC) in vitro with one or more antigens (including antigenic portions thereof, such as epitope(s), or a cell) of the cancer, which can be optionally expressed from a vector, such as an human leukocyte antigen A2 (HLA-A2) binding peptide, e.g., 0.3 M MART-1 (26-35, 27 L) or gp100 (209-217, 210M), in the presence of a T-cell growth factor, such as IL-2 or IL-15 (e.g., at about 300 IU/ml), with IL-2 being preferred. Methods of expanding γδ T-cells have been described, for example, in WO2017035375 and WO2011053750; the contents of which are expressly incorporated by reference herein.

The γδ T-cells can also be derived from human induced pluripotent stem cells (hiPSCs). The pluripotent stem cells can, for example, be isolated from the patient having the cancer. In other aspects, the pluripotent stem cells may be isolated from a source other than the patient with cancer. The optionally enriched and/or optionally expanded compositions comprising γδ T-cells can also comprise natural killer (NK) cells and optionally further comprise other immunocompetent cells including but not limited to monocytes, macrophages and dendritic cells. Methods for generating γδ T-cells from induced pluripotent stem cells has been described, for example, in Watanabe et al. 2017, Stem Cells Transl Med 7(1): 34-44 and Zeng et al. (2019), PLoS One 14(5): e0216815; the contents of each of which are expressly incorporated by reference herein.

The terms “isolated’ and “isolated population” of cells as used herein refers to a cell or a plurality of cells removed from the tissue or state in which they are found in a subject. The terms may further include cells that have been separated according to such parameters as, but not limited to, cell surface markers, a reporter marker such as a dye or label.

The term “expressed” or “expression” and the like as used herein has its ordinary meaning in the art and refers to the transcription from a gene to give an RNA nucleic acid molecule at least complementary in part to a region of one of the two nucleic acid strands of the gene. The term “expressed” or “expression” as used herein also refers to the translation from said RNA nucleic acid molecule to give a protein, a polypeptide, or a portion or fragment thereof.

The term “vector” as used herein refers to a polynucleotide comprised of single strand, double strand, circular, or supercoiled DNA or RNA. A typical vector may be comprised of the following elements operatively linked at appropriate distances for allowing functional gene expression; replication origin, promoter, enhancer, 5′ mRNA leader sequence, ribosomal binding site, nucleic acid cassette, termination and polyadenylation sites, and selectable marker sequences. One or more of these elements may be omitted in specific applications. The vector may also contain a nucleic acid cassette, which can include a restriction site for insertion of the nucleic acid sequence to be expressed. In a functional vector the nucleic acid cassette contains the nucleic acid sequence to be expressed including translation initiation and termination sites. A vector is constructed so that the particular coding sequence (for example, a coding sequence for a CAR of the present disclosure) is located in the vector with the appropriate control sequences, the positioning and orientation of the coding sequence with respect to the control sequences being such that the coding sequence is operably linked and/or is transcribed “under the control” of the control sequences. Modification of the sequences encoding the particular protein of interest may be desirable to achieve this end. For example, in some cases it may be necessary to modify the sequence so that it may be operably linked to the control sequences with the appropriate orientation or to maintain the reading frame. The control sequences and/or other regulatory sequences may be ligated to the coding sequence prior to insertion into a vector. Alternatively, the coding sequence can be cloned directly into an expression vector that already contains the control sequences and an appropriate restriction site that is in reading frame with and under regulatory control of the control sequences. The invention includes a vector comprising a nucleic acid sequence encoding a non-signaling CAR as described herein. A non-limiting example of a vector is an AAV, a lentiviral vector, or a retroviral vector. In certain aspects, the invention is a vector that comprises a nucleic acid encoding a non-signaling CAR as described herein. A specific example of a vector is the Baboon envelope pseudotyped lentiviral vector (BaEv). In certain aspects, the vector further comprises a nucleic acid that encodes a survival factor. In yet further aspects, the vector comprises a nucleic acid that encodes IL15, IL2 and/or IL7. In additional aspects, a lentiviral vector comprises a nucleic acid that encodes non-signaling CAR as described herein. The lentiviral vector can further comprise a nucleic acid that encodes a survival factor. In another aspect, the lentiviral vectors can further comprise a nucleic acid that encodes IL15, IL2 and/or IL7.

The term “promoter” as used herein refers to the DNA sequence that determines the site of transcription initiation from an RNA polymerase. A “promoter-proximal element” may be a regulatory sequence within about 200 base pairs of the transcription start site.

The term “recombinant cell” refers to a cell that has a new combination of nucleic acid segments that are not covalently linked to each other in nature. A new combination of nucleic acid segments can be introduced into an organism using a wide array of nucleic acid manipulation techniques available to those skilled in the art. A recombinant cell can be a single eukaryotic cell, or a single prokaryotic cell, or a mammalian cell. The recombinant-cell may harbor a vector that is extragenomic. An extragenomic nucleic acid vector does not insert into the cell's genome. A recombinant cell may further harbor a vector or a portion thereof that is intragenomic. The term “intragenomic” defines a nucleic acid construct incorporated within the recombinant-cell's genome.

The terms “recombinant nucleic acid” and “recombinant DNA” as used herein refer to combinations of at least two nucleic acid sequences that are not naturally found in a eukaryotic or prokaryotic cell. The nucleic acid sequences include, but are not limited to, nucleic acid vectors, gene expression regulatory elements, origins of replication, suitable gene sequences that when expressed confer antibiotic resistance, protein-encoding sequences, and the like. The term “recombinant” with respect to a protein or peptide is meant to include a polypeptide or protein produced by recombinant DNA techniques such that it is distinct from a naturally occurring polypeptide either in its location, purity or structure. Generally, such a recombinant polypeptide or protein will be present in a cell in an amount different from that normally observed in nature.

The terms “operably” or “operatively linked” as used herein refer to the configuration of the coding and control sequences so as to perform the desired function. Thus, control sequences operably linked to a coding sequence are capable of effecting the expression of the coding sequence. A coding sequence is operably linked to or under the control of transcriptional regulatory regions in a cell when DNA polymerase will bind the promoter sequence and transcribe the coding sequence into mRNA that can be translated into the encoded protein. The control sequences need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence.

The terms “heterologous” and “exogenous” as they relate to nucleic acid sequences such as coding sequences and control sequences denote sequences that are not normally associated with a region of a recombinant construct or with a particular chromosomal locus, and/or are not normally associated with a particular cell. Thus, a “heterologous” region of a nucleic acid construct is an identifiable segment of nucleic acid within or attached to another nucleic acid molecule that is not found in association with the other molecule in nature. For example, a heterologous region of a construct could include a coding sequence flanked by sequences not found in association with the coding sequence in nature. Another example of a heterologous coding sequence is a construct where the coding sequence itself is not found in nature (e.g., synthetic sequences having codons different from the native gene). Similarly, a cell transformed with a construct, which is not normally present in the host cell, would be considered heterologous for purposes of this invention.

The promoter can be modified by the addition or deletion of sequences, or replaced with alternative sequences, including natural and synthetic sequences as well as sequences that may be a combination of synthetic and natural sequences. Many eukaryotic promoters contain two types of recognition sequences: the TATA box and the upstream promoter elements. The former, located upstream of the transcription initiation site, is involved in directing RNA polymerase to initiate transcription at the correct site, while the latter appears to determine the rate of transcription and is upstream of the TATA box. Enhancer elements can also stimulate transcription from, linked promoters, but many function exclusively in a particular cell type. Many enhancer/promoter elements derived from viruses, e.g., the SV40, the Rous sarcoma virus (RSV), and CMV promoters are active in a wide array of cell types, and are termed “constitutive” or “ubiquitous.” The nucleic acid sequence inserted in the cloning site may have any open reading frame encoding a polypeptide of interest, with the proviso that where the coding sequence encodes a polypeptide of interest, it should lack cryptic splice sites that can block production of appropriate mRNA molecules and/or produce aberrantly spliced or abnormal mRNA molecules.

The termination region that is employed primarily will be one of convenience, since termination regions appear to be relatively interchangeable. The termination region may be native to the intended nucleic acid sequence of interest, or may be derived from another source.

The term “targeted therapy”, as used herein, refers to any therapeutic molecule that targets any aspect of the immune system.

The terms “transformation”, “transduction” and the like denote the introduction of a polynucleotide into a recipient-cell or cells.

The invention includes an engineered γδ T-cell that expresses a non-signaling chimeric antigen receptor (CAR) and that further express a survival factor, wherein the survival factor is a DNA, RNA, or polypeptide that confers resistance to a chemotherapeutic agent. The invention additionally provides an engineered γδ T-cell that comprises a non-signaling chimeric antigen receptor (CAR) and wherein the γδ T cell further expresses a cytokine selected from the group consisting of IL-15, IL-2, and/or IL-7. The invention further provides an engineered γδ T-cell that comprises a non-signaling chimeric antigen receptor (CAR), wherein the CAR comprises only one co-stimulatory domain.

As described above, the antigen binding domain of the non-signaling CAR binds a tumor antigen. The term “tumor antigen,” “tumor associated antigen” and “TAA” are used interchangeably herein. Non-limiting examples of tumor-associated antigens include CD19; CD123; CD22; CD30; CD171; CS-1 (also known as CD2 subset 1, CRACC, SLAMF7, CD319, and 19A 24); c-type lectin-like molecule-1 (CLL-1 or CLECL 1); CD33; epidermal growth factor receptor variant III (EGFRvIII); ganglioside G2 (GD 2); ganglioside GD3 (aNeu 5Ac (2-8) aNeu5Ac (2-3) bDGalp (1-4) bDGlcp (1-1) Cer); TNF receptor family member B Cell Maturation (BCMA); tn antigen ((TnAg) or (GalNAc. Alpha.-Ser/Thr)); prostate Specific Membrane Antigen (PSMA); receptor tyrosine kinase-like orphan receptor 1 (ROR 1); fms-like tyrosine kinase 3 (FLT 3); tumor associated glycoprotein 72 (TAG 72); CD38; CD44v6; carcinoembryonic antigen (CEA); epithelial cell adhesion molecule (EPCAM); B7H3 (CD 276); KIT (CD 117); interleukin-13 receptor subunit alpha-2 (IL-13 Ra2 or CD213 A2); mesothelin; interleukin 11 receptor alpha (IL-11 Ra); prostate Stem Cell Antigen (PSCA); protease serine 21 (Testisin or PRSS 21); vascular endothelial growth factor receptor 2 (VEGFR 2); a Lewis (Y) antigen; CD24; platelet-derived growth factor receptor beta (PDGFR-beta); stage specific embryonic antigen-4 (SSEA-4); CD20; a folate receptor alpha (folate receptor 1: FOLR1); receptor tyrosine protein kinase ERBB2 (Her 2/neu); mucin 1, cell surface associated material (MUC 1); epidermal Growth Factor Receptor (EGFR); neural Cell Adhesion Molecule (NCAM); a Prostase; prostatic Acid Phosphatase (PAP); mutant elongation factor 2 (ELF 2M); ephrin B2; fibroblast activation protein alpha (FAP); insulin-like growth factor 1 receptor (IGF-I receptor), carbonic Anhydrase IX (CAIX); proteasome (Prosome, macropain) subunit beta type 9 (LMP 2); glycoprotein 100 (gp 100); an oncogene fusion protein (BCR-Abl) consisting of the Breakpoint Cluster Region (BCR) and the homolog 1 (Abl) of the Abelson murine leukemia virus oncogene; a tyrosinase enzyme; ephrin type a receptor 2 (EphA 2); fucose GM1; sialic acid Lewis adhesion molecule (sLe); ganglioside GM3 (aNeu 5Ac (2-3) bDGalp (1-4) bDGlcp (1-1) Cer); transglutaminase 5 (TGS 5); high Molecular Weight Melanoma Associated Antigen (HMWMAA); o-acetyl-GD 2 ganglioside (OAcGD 2); folate receptor 3; tumor endothelial marker 1 (TEM 1/CD 248); tumor endothelial marker 7 related (TEM 7R); claudin 6 (CLDN 6); thyroid Stimulating Hormone Receptor (TSHR); a G protein-coupled receptor class C group 5D member (GPRC 5D); chromosome X open reading frame 61 (CXORF 61); CD97; CD 179a; anaplastic Lymphoma Kinase (ALK); polysialic acid; placenta-specific (PLACENTA-specific) 1 (PLAC 1); the hexasaccharide moiety (globoH) of the globoH glycoceramide (glycoceramide); mammary gland differentiation antigen (NY-BR-1); uroplakin 2 (UPK 2); hepatitis a virus cell receptor 1 (HAVCR 1); adrenergic receptor beta 3 (ADRB 3); pannexin 3 (PANX 3); g protein-coupled receptor 20 (GPR 20); lymphocyte antigen 6 complex, locus K9 (LY 6K); olfactory receptor 51E2 (OR 51E 2); TCR 7 alternate reading frame protein (TARP); wilms tumor protein (WT 1); cancer/testis antigen 1 (NY-ESO-1); cancer/testis antigen 2 (LAGE-1 a); melanoma-associated antigen 1 (MAGE-A1); ETS translocation-variant gene 6 (ETV 6-AML) located on chromosome 12 p; sperm protein 17 (SPA 17); x antigen family member 1A (XAGE 1); angiogenin binds to cell surface receptor 2 (Tie 2); melanoma cancer testis antigen-1 (MAD-CT-1); melanoma cancer testis antigen-2 (MAD-CT-2); fos-related antigen 1; tumor protein p53 (p 53); a p53 mutant; a prostein; survival (surviving); a telomerase; prostate cancer tumor antigen-1 (PCTA-1 or Galectin 8 (Galectin 8)), melanoma antigen 1 recognized by T cells (Melana or MART 1); rat sarcoma (Ras) mutant; human telomerase reverse transcriptase (hTERT); a sarcoma translocation breakpoint; melanoma apoptosis inhibitors (ML-IAP); ERG (transmembrane protease, serine 2 (TMPRSS 2) ETS fusion gene); n-acetylglucosaminyltransferase V (NA 17); paired box protein Pax-3 (PAX 3); an androgen receptor; cyclin B1; v-myc avian myelomatosis virus oncogene neuroblastoma derived homolog (MYCN); ras homolog family member C (RhoC); tyrosinase-related protein 2 (TRP-2); cytochrome P450B 1 (CYP 1B 1); CCCTC-binding factor (zinc finger protein)—like (BORIS or Brother of the Regulator of Imprinted Sites), squamous cell carcinoma antigen recognized by T cells 3 (SART 3); paired box protein Pax-5 (PAX 5); the preproceptorin (proacrosin) binding protein sp32 (OY-TES 1); lymphocyte-specific protein tyrosine kinase (LCK); kinase anchoring protein 4 (AKAP-4); synovial sarcoma X breakpoint 2 (SSX 2); receptor for advanced glycation end products (RAGE-1); renal ubiquitin 1 (renal ubiquitin 1) (RU 1); renal ubiquitin 2 (RU 2); legumain; human papillomavirus E6 (UPV E6); human papilloma virus E7 (UPV E7); an intestinal carboxylesterase; mutated heat shock protein 70-2 (mut hsp 70-2); CD79a; CD79b; CD72; leukocyte-associated immunoglobulin-like receptor 1 (LAIR 1); an Fc fragment of IgA receptor (FCAR or CD 89); leukocyte immunoglobulin-like receptor subfamily a member 2 (LILRA 2); CD300 molecule-like family member f (CD 300 LF); c-type lectin domain family 12 member a (CLEC 12A); bone marrow stromal cell antigen 2 (BST 2); mucin-like hormone receptor-like protein 2 (EMR 2) containing an EGF-like module; lymphocyte antigen 75 (LY 75); glypican-3 (Glypican-3, GPC3); fc receptor-like protein 5 (FCRL 5); and immunoglobulin lambda-like polypeptide 1 (IGLL 1). In certain aspects, the tumor antigen includes EphA2, B cell maturation antigen (BCMA), B7-H3, B7-H6, CAIX, CA9, CD22, CD19, CD20, ROR1, kappa or light chain, carcinoembryonic antigen, alpha-fetoprotein, CA-125, Glypican-3, epithelial tumor antigen, melanoma-associated antigen, EGP2, EGP40, EPCAM, ERBB3, ERBB4, ErbB3/4, PAP, FAR, FBP, fetal AchR, Folate Receptor a, mutated p53, mutated ras, HER2, ERBB2, HER3, folate binding protein, HIV-1 envelope glycoprotein gp120, HIV-1 envelope glycoprotein gp41, 5T4, 8H9, GD2, CD123, CD171, CS-1, CD23, CD24, CD33, CD30, CD38, CD56, c-Met, fap, mesothelin, GD3, HERV-K, IL-11Ra, IL-13Ra, IL-13Ra2, CSPG4, Lewis-Y, MCSP, Mucl, Muc16, NCAM, NKG2D ligands, NY-ESO-1, PRAME, PSCA, PSC1, PSMA, EGFR, Spl7, SURVIVIN, TAG72, TEM1, TEM8, epidermal growth factor receptor variant III, EGFRvIII, VEGFR2. In yet additional aspects, the tumor antigen is an NKG2D ligand selected from the group consisting of ULBP-1, ULBP-2, ULBP-3, ULBP-4, ULBP-5, ULBP-6, MIC-A, and MIC-B.

In certain specific aspects, the tumor antigen is selected from the group consisting of CD19, CD33, CD123, CD20, CD22, EGFRvIII, IL13ra2, folate receptor 1 (FOLR1), and mesothelin. In certain additional aspects, the tumor antigen is selected from the group consisting of CD19, CD33, CD123, CD20, and CD22. In yet further aspects, the tumor antigen is selected from the group consisting of CD19, CD33, and CD123.

In specific aspects, the tumor antigen is CD19. The human CD19 antigen is a 95 kd transmembrane glycoprotein belonging to the immunoglobulin superfamily. CD19 is expressed early in B-cell differentiation and is only lost at terminal B-cell differentiation into plasma cells. Consequently, CD19 is expressed on all B-cell malignancies except for multiple myeloma. A non-signaling CAR that binds CD19 can be a monoCAR or a dual CAR.

In yet additional aspects, the tumor antigen is CD33. CD33 is a myeloid differentiation antigen and is highly expressed on myeloid progenitor cells but is expressed at low levels in differentiated myeloid cells (i.e., macrophages and granulocytes). CD33 has been reported to be expressed in about 88% of Acute Myeloid Leukemia (AML) (Ehniger et al (2014), Distribution and levels of cell surface expression of CD33 and CD123 in acute myeloid leukemia, Blood Cancer Journal 4(6): e 218; the contents of which are expressly incorporated by reference herein). A non-signaling CAR that binds CD33 can be a monoCAR or a dual CAR; for example, the dual CAR can bind CD33 and CD123.

CD123 is the interleukin 3 receptor alpha chain (IL-3a) and is overexpressed on AML tumor cells as compared to normal hematopoietic cells.

In additional aspects, the tumor antigen is selected from the group consisting of CD19 and CD33. In yet additional aspects, the CAR is a monoCAR and binds a tumor antigen selected from CD19 and CD33. In yet additional aspects, the CAR is a dualCAR (e.g, a tandem dualCAR or a separate dualCAR) that binds CD19 and a second tumor antigen. In further aspect, the CAR is a dualCAR (e.g, a tandem dualCAR or a separate dualCAR) that binds CD33 and a second tumor antigen, for example, CD123.

The antigen binding domain can comprise an antibody or an antigen binding molecule thereof with specificity for the tumor antigen; for example, an anti-CD19 antibody or antigen binding fragment thereof, an anti-CD33 antibody or antigen binding fragment thereof, and/or an anti-CD123 antibody or antigen-binding fragment thereof. Antibodies can include, for example, monoclonal antibodies, recombinantly produced antibodies, monospecific antibodies, multi specific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, antibody fusions (sometimes referred to herein as “antibody conjugates”), heteroconjugate antibodies, single domain antibodies, monovalent antibodies, single chain antibodies or single-chain Fvs (scFv), camelized antibodies, affibodies, Fab fragments, F(ab′)₂ fragments, disulfide-linked Fvs (sdFv), anti-idiotypic (anti-Id) antibodies (including, e.g., anti-anti-Id antibodies), minibodies, domain antibodies, synthetic antibodies (sometimes referred to herein as “antibody mimetics”), and antigen-binding fragments of any of the above. An “antigen binding molecule,” “antigen binding portion,” “antibody fragment,” or “antigen-binding fragment” refers to any molecule that comprises the antigen binding parts (e.g., CDRs) of the antibody from which the molecule is derived. An antigen binding molecule can include the antigenic complementarity determining regions (CDRs). Examples of antibody fragments include, but are not limited to, Fab, Fab′, F(ab′)₂, and Fv fragments, dAb, linear antibodies, scFv, and multi specific antibodies formed from antigen binding molecules.

A scFv refers to a fusion protein of the variable regions of the heavy (VH) and light chains (VL) of an immunoglobulin, connected with a short linker peptide (generally of about 10 to about 25 amino acids) to produce a VH-linker-VL structure (also referred to an antigen binding domain). The linker is usually rich in glycine for flexibility, as well as serine or threonine for solubility, and can either connect the N-terminus of the VH with the C-terminus of the VL, or vice versa. This scFv retains the specificity of the original immunoglobulin, despite removal of the constant regions and the introduction of the linker.

In certain embodiments, the antigen recognition domain is a multivalent scFv. A multivalent scFv comprises two immunoglobulin derived antigen binding domains (such as a VH-linker-VL chain) joined by an additional linker, wherein the antigen binding domain recognize different antigens or different portions of the same antigen. In certain aspects, one of the antigen binding domains recognizes a tumor associated antigen. In certain aspects, one of the antigen binding domains recognizes an activation molecule on the target cell. In certain aspects, one of the antigen binding domains recognizes an antigen that is increased in expression on the target cell as a result of the additional therapeutic treatment (such as, but not limited to, a stress induced antigen).

In certain embodiments, the antigen recognition domain is a bivalent scFv. Such bivalent scFv may have the structure VHᵢ-linkerₐ-VLᵢ-VH-linker_{b}-VL-linker_{c}-VH-linker_{d}-VL as an example (with the understanding that the VH and VL may be arranged in different orientations as discussed above). The linker peptide should be flexible enough to allow the antigen binding domain to adopt conformations suitable for antigen recognition and binding. The simplest form is the hinge region from IgGl. A variety of linker peptides may be used in conjunction with the CARs described herein.

In certain aspects, the extracellular antigen binding domain comprises an anti-CD19 antibody or antigen-binding fragment thereof. In additional aspects, the extracellular antigen binding domain comprises an anti-CD33 antibody or antigen-binding fragment thereof. In yet further aspects, the extracellular antigen binding domain comprises an anti-CD123 antibody or antigen-binding fragment thereof. In certain aspects, the antigen binding domain comprising an scFv, for example, an anti-CD19 scFv an anti-CD33 scFv, or an anti-CD123 scFv. For example, the anti-CD19 scFv can be derived from FMC63 murine antibody, which is the scFv used in KYMRIAH and YESCARTA. In another example, the anti-CD33 scFv can be derived from gemtuzumab ozogamicin (GO) (hP67.6), vadastuximab talirine (SGN-CD33A), lintuzumab, M195, or MY96 (Mylotarg). MY96 has been described, for example, in WO2016014576 and Kenderian et al. (2015), Leukemia 29: 1637-1647.

The antigen-binding domain can also comprise any molecule (recognition element) that can bind to a tumor antigen, e.g., peptide that specifically binds to the tumor antigen or a receptor ligand that binds to the tumor antigen. Examples of suitable soluble receptor ligands include autocrine and paracrine growth factors, chemokines, cytokines, hormones, and engineered artificial small molecule ligands that exhibit the required specificity. Natural ligand sequences can also be engineered or optimized to increase their specificity for a particular target cell. In certain examples, the extracellular antigen domain can comprise an IL3 molecule that specifically binds CD123. An IL3 molecule that specifically binds CD123 can be a natural ligand of CD123 or a variant or fragment thereof that specifically binds to CD123. In specific aspects, the IL3 molecule is the full-length, wild-type human IL3 (hIL3; UniProt P08700). A CD123 binding fragment of an IL-3, is any portion of the protein that binds to CD123 (such as the alpha subunit and/or beta subunit of CD123). For example, the IL3 molecule is a binding fragment of human IL3 that binds to CD123. In some embodiments, the CD123-binding fragment of an IL3 molecule includes any naturally occurring, enzymatically obtainable, synthetic, or genetically engineered polypeptide fragment of IL-3 that specifically binds CD123 to form a complex. In some embodiments, the CD123-binding fragment of IL-3 includes at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% or more of a naturally occurring IL-3, e.g., a naturally occurring human IL3, such that the fragment retains the ability to bind CD 123. Non-limiting examples of IL3 molecules and fragments thereof are described in WO2023010126, Perriello et al. (2023) Blood Adv 7(12): 2855-2871, and Boucher et al. (2023), Molecular Therapy Oncology 31(100751); the contents of which are expressly incorporated by reference herein.

In certain aspects, the dualCAR (e.g., a tandem dualCAR or a separate dualCAR) comprises an antibody or fragment thereof (e.g., an scFv) that binds to one tumor antigen, such as CD19 or CD33, and a recognition element other than an antibody or fragment thereof (a non-antibody recognition element) that binds to a second tumor antigen. In certain aspects, the non-antibody recognition element is a peptide. An exemplary dual CAR comprises an anti-CD33 scFv and an IL3 molecule. In another example the dualCAR is a tandem dualCAR that comprises an anti-CD33 scFv and hIL3.

In certain additional aspects, the dualCAR (e.g, a tandem dualCAR or a separate dualCAR) comprises an antibody or fragment thereof (e.g., an scFv) that binds to one tumor antigen, such as CD19 or CD33, and a second antibody or fragment thereof that binds to a second tumor antigen. Such an exemplary dual CAR comprises an anti-CD33 scFv and an anti-CD123 scFv. Additional examples of a dualCAR is a tandem dualCAR that comprises an anti-CD33 scFv and an anti-CD123 scFv, a tandem dualCAR that comprises an anti-CD19 scFv and an anti-CD20 scFv, and a tandem dualCAR that comprises an anti-CD33 scFv and an anti-CD22 scFv.

The non-signaling CARs described herein can further comprise a transmembrane domain, and optionally at least one co-stimulatory domain, wherein the non-signaling CAR does not comprising a T-cell activation domain (e.g., CD3z). Also, the non-signaling CARs described herein can further comprise a transmembrane domain, a hinge domain, and optionally at least one co-stimulatory domain, wherein the non-signaling CAR does not comprising a T-cell activation domain (e.g., CD3z). In certain aspects, the non-signaling CAR comprises a transmembrane domain, a hinge domain, and only one co-stimulatory domain, and does not include an intracellular T-cell activation domain. The non-signaling CARs in accordance with the invention can, for example, have the following structure: i) an extracellular domain (also referred to herein as an “ectodomain”) comprising an antigen recognition domain/moiety that binding a tumor antigen such as CD19 or CD33, and optionally binding to a second tumor antigen, ii) a hinge domain (that attaches the extracellular domain to the transmembrane domain); iii) a transmembrane domain and iv) an optional co-stimulatory domain, and iv) wherein the endodomain that does not include a T-cell activation domain such as CD3z. The non-signaling CARs in accordance with the invention can have the following structure: i) an extracellular domain comprising an antigen recognition domain/moiety that binds a tumor antigen such as CD19 or CD33, and optionally binding to a second tumor antigen, ii) a hinge domain; iii) a transmembrane domain and iv) at least one co-stimulatory domain, and v) an endodomain that does not include a T-cell activation domain such as CD3z. The non-signaling CARs in accordance with the invention can additionally have the following structure: i) an extracellular domain comprising an antigen recognition domain/moiety that binding a tumor antigen such as CD19 or CD33, and optionally binding to a second tumor antigen, ii) a hinge domain; iii) a transmembrane domain and iii) only one co-stimulatory domain, and iv) an endodomain that does not include a T-cell activation domain. In certain aspects, the γδ T cell expressing the nsCAR further expresses a cytokine selected from the group consisting of IL15, IL2 and IL7. In yet additional aspects, the γδ T cell expressing the nsCAR further expresses IL15; the IL15 can be secreted or membrane-bound. In additional aspects, the gd T cell further expresses a survival factor.

In certain specific aspects, the non-signaling CARs in accordance with the invention is a monoCAR having the following structure: i) an extracellular domain comprising an antigen recognition domain/moiety that binds CD19, ii) a hinge domain; iii) a transmembrane domain and iv) at least one co-stimulatory domain, and v) an endodomain that does not include a T-cell activation domain. The non-signaling CARs in accordance with the invention can additionally be a monoCAR having the following structure: i) an extracellular domain (also referred to herein as an “ectodomain”) comprising an antigen recognition domain/moiety that binding a tumor antigen such as CD19, ii) a hinge domain; iii) a transmembrane domain and iv) only one co-stimulatory domain, and v) an endodomain that does not include a T-cell activation domain. In certain aspects, the γδ T cell expressing the nsCAR further expresses a cytokine selected from the group consisting of IL15, IL2 and IL7. In yet additional aspects, the γδ T cell expressing the nsCAR further expresses IL15; the IL15 can be secreted or membrane-bound. In additional aspects, the gd T cell further expresses a survival factor.

In additional aspects, the non-signaling CARs in accordance with the invention is a tandem dualCAR having the following structure: i) an extracellular domain comprising an antigen recognition domain/moiety that binds CD19 and a second tumor antigen, such as CD20 or CD22; ii) a hinge domain; iii) a transmembrane domain and iv) at least one co-stimulatory domain, and v) an endodomain that does not include a T-cell activation domain. The non-signaling CARs in accordance with the invention can additionally be a tandem dualCAR having the following structure: i) an extracellular domain (also referred to herein as an “ectodomain”) comprising an antigen recognition domain/moiety that binding CD19 and a second tumor antigen, such as CD20 or CD22; ii) a hinge domain; iii) a transmembrane domain and iv) only one co-stimulatory domain, and v) an endodomain that does not include a T-cell activation domain. In certain aspects, the γδ T cell expressing the nsCAR further expresses a cytokine selected from the group consisting of IL15, IL2 and IL7. In yet additional aspects, the γδ T cell expressing the nsCAR further expresses IL15; the IL15 can be secreted or membrane-bound. In additional aspects, the gd T cell further expresses a survival factor.

In yet additional aspects, the non-signaling CAR in accordance with the invention is separate dualCAR having a first CAR and a second CAR, wherein the first CAR has the following structure: i) an extracellular domain comprising an antigen recognition domain/moiety that binds CD19, ii) a hinge domain; iii) a transmembrane domain and iv) at least one co-stimulatory domain, and v) an endodomain that does not include a T-cell activation domain; and wherein the second CAR has the following structure: i) an extracellular domain comprising an antigen recognition domain/moiety that binds a second tumor antigen such as CD20, or CD22, ii) a hinge domain; iii) a transmembrane domain and iv) at least one co-stimulatory domain, and v) an endodomain that does not include a T-cell activation domain. The non-signaling CARs in accordance with the invention can additionally be a separate dualCAR having a first CAR and a second CAR, wherein the first CAR has the following structure: i) an extracellular domain comprising an antigen recognition domain/moiety that binds CD19, ii) a hinge domain; iii) a transmembrane domain and iv) only one co-stimulatory domain, and v) an endodomain that does not include a T-cell activation domain; and wherein the second CAR has the following structure: i) an extracellular domain comprising an antigen recognition domain/moiety that binds a second tumor antigen such as CD20, or CD22, ii) a hinge domain; iii) a transmembrane domain and iv) only one co-stimulatory domain, and v) an endodomain that does not include a T-cell activation domain. In certain aspects, the γδ T cell expressing the nsCAR further expresses a cytokine selected from the group consisting of IL15, IL2 and IL7. In yet additional aspects, the γδ T cell expressing the nsCAR further expresses IL15; the IL15 can be secreted or membrane-bound. In additional aspects, the gd T cell further expresses a survival factor.

In certain specific aspects, the non-signaling CARs in accordance with the invention is a monoCAR having the following structure: i) an extracellular domain (also referred to herein as an “ectodomain”) comprising an antigen recognition domain/moiety that binds CD33, ii) a hinge domain; iii) a transmembrane domain and iii) at least one co-stimulatory domain, and iv) an endodomain that does not include a T-cell activation domain. The non-signaling CARs in accordance with the invention can additionally be a monoCAR having the following structure: i) an extracellular domain (also referred to herein as an “ectodomain”) comprising an antigen recognition domain/moiety that binding CD33, ii) a hinge domain; iii) a transmembrane domain and iii) only one co-stimulatory domain, and iv) an endodomain that does not include a T-cell activation domain. In certain aspects, the γδ T cell expressing the nsCAR further expresses a cytokine selected from the group consisting of IL15, IL2 and IL7. In yet additional aspects, the γδ T cell expressing the nsCAR further expresses IL15; the IL15 can be secreted or membrane-bound. In additional aspects, the gd T cell further expresses a survival factor.

In an addition aspects, the non-signaling CARs in accordance with the invention is a tandem dualCAR having the following structure: i) an extracellular domain (also referred to herein as an “ectodomain”) comprising an antigen recognition domain/moiety that binds CD33 and a second tumor antigen such as CD123, ii) a hinge domain; iii) a transmembrane domain and iii) at least one co-stimulatory domain, and iv) an endodomain that does not include a T-cell activation domain. The non-signaling CARs in accordance with the invention can additionally be a dualCAR having the following structure: i) an extracellular domain (also referred to herein as an “ectodomain”) comprising an antigen recognition domain/moiety that binding CD33 and a second tumor antigen, such as CD123, ii) a hinge domain; iii) a transmembrane domain and iii) only one co-stimulatory domain, and iv) an endodomain that does not include a T-cell activation domain. In certain aspects, the γδ T cell expressing the nsCAR further expresses a cytokine selected from the group consisting of IL15, IL2 and IL7. In yet additional aspects, the γδ T cell expressing the nsCAR further expresses IL15; the IL15 can be secreted or membrane-bound. In additional aspects, the gd T cell further expresses a survival factor.

In a further aspect, the non-signaling CAR in accordance with the invention is separate dualCAR having a first CAR and a second CAR, wherein the first CAR has the following structure: i) an extracellular domain comprising an antigen recognition domain/moiety that binds CD33, ii) a hinge domain; iii) a transmembrane domain and iii) at least one co-stimulatory domain, and iv) an endodomain that does not include a T-cell activation domain; and wherein the second CAR has the following structure: i) an extracellular domain comprising an antigen recognition domain/moiety that binds a second tumor antigen such as CD123, ii) a hinge domain; iii) a transmembrane domain and iii) at least one co-stimulatory domain, and iv) an endodomain that does not include a T-cell activation domain. The non-signaling CARs in accordance with the invention can additionally be a separate dualCAR having a first CAR and a second CAR, wherein the first CAR has the following structure: i) an extracellular domain comprising an antigen recognition domain/moiety that binds CD33, ii) a hinge domain; iii) a transmembrane domain and iii) only one co-stimulatory domain, and iv) an endodomain that does not include a T-cell activation domain; and wherein the second CAR has the following structure: i) an extracellular domain comprising an antigen recognition domain/moiety that binds a second tumor antigen such as CD123, ii) a hinge domain; iii) a transmembrane domain and iii) only one co-stimulatory domain, and iv) an endodomain that does not include a T-cell activation domain. In certain aspects, the γδ T cell expressing the nsCAR further expresses a cytokine selected from the group consisting of IL15, IL2 and IL7. In yet additional aspects, the γδ T cell expressing the nsCAR further expresses IL15; the IL15 can be secreted or membrane-bound. In additional aspects, the gd T cell further expresses a survival factor.

In certain embodiments, a peptide linker from 1 to 30 amino acids can be present in the CAR to separate the various domains of the CAR. In yet other aspects, the peptide linker is less than 15 amino acids in length. For example, a peptide linker can be present between the antigen recognition domain/moiety and other domains which may be present in the extracellular domain, between the antigen recognition domain/extracellular domain and the hinge domain, between the hinge domain and the transmembrane domain, or between the transmembrane domain and the intracellular signaling domain. A peptide linker can be present between all domains or only between a portion of the domains/moieties. Furthermore, when the endodomain comprises more than one element, a linker peptide may be present between some or all of the individual elements in the endodomain. Each linker peptide in the CAR can be the same or can be different. An exemplary linker peptide can be 30 amino acids in length or less, 20 amino acids in length or less, or 15 amino acids in length or less. Non-limiting examples of such linker peptides are FLAG, influenza virus haemagglutinin (HA), c-myc, polyHis; Strep tags, Strep II tags, FLAG tags, glutathione S-transferase (GST) tags, green fluorescent protein (GFP) tags, hemagglutinin A (HA) tags, histidine (His) tags, luciferase tags, maltose-binding protein (MBP) tags, c-Myc tags, protein A tags, protein G tags, a human serum albumin (HSA), or influenza virus haemagglutinin. In certain aspects, the peptide linker is c-myc (for example, having the amino acid sequence of EQKLISEEDL (SEQ ID NO: 1) or FLAG (for example, having the amino acid sequence of DYKDDDDK (SEQ ID NO:2). Another example of a linker peptide is (GSSS)ₙ, wherein n is an integer from 1 to 10. In yet another embodiment, the linker peptide is HA, for example, having an amino sequence of GLFGAIAGFIENG (SEQ ID NO: 3) or EGMIDGWYG (SEQ ID NO: 4).

The intracellular signaling domain or endodomain of the CAR is responsible for activation of at least one of the normal effector functions of the host cell in which the CAR is placed. The term “effector function” refers to a specialized function of a differentiated cell. In a conventional signaling CAR, the intracellular signaling domain is responsible for activation of at least one of a normal immune effector function. Immune effector function of a T-cell, for example, may be cytolytic activity or helper activity, including, but not limited to, the secretion of cytokines. The endodomain allows transmission of a signal after antigen binding. After antigen recognition, receptors cluster and a signal is transmitted to the immune system cell (e.g., the gd T-cell) containing the CAR. In conventional signaling CARs, the endodomain comprises at least one signaling domain, such as the T-cell activation domain and can optionally comprising one or more co-stimulatory domains. The most commonly used activation domain is that of CD3-zeta, which contains three immunoreceptor tyrosine-based activation motifs (ITAMs). “First-generation” CARs typically have the endodomain from the CD3z, which is the primary transmitter of signals from endogenous TCRs. “Second-generation” CARs add an intracellular signaling domain from various costimulatory protein receptors (e.g., CD28, 41BB, DAP10, OX40 or ICOS) to the cytoplasmic tail of the endodomain to provide additional signals to the T cell. “Third-generation” CARs have an endodomain that combine multiple signaling domains, such as CD3-zeta-CD28-41BB or CD3-zeta-CD28-OX40, to further augment potency.

The non-signaling CARs of the present invention do not include a T-cell activation domain, such as a CD3-zeta T-cell activation domain, in the endodomain. The non-signaling CAR can include one or more co-stimulatory domains (e.g., CD28, 41BB, DAP10, OX40 or ICOS). In certain aspects, the non-signaling CAR includes only one co-stimulatory domain. Non-limiting examples of co-stimulatory domains include CD28, CD27, 4-IBB, DAP-10, OX40, and combinations thereof, as well as other similar molecules and fragments as well as mutations to the foregoing, such as modifying the immunoreceptor tyrosine-based activation motif(s) (ITAMs). In certain embodiments, the costimulatory domain is a functional signaling domain from 41BB, OX40 and/or CD28. In certain aspects, the intracellular signaling domain or endodomain can comprise a sequence encoding a costimulatory signaling domain, and does not comprise a sequence encoding a T-cell activation domain. In certain embodiments, the endodomain of the non-signaling CAR comprises a CD28 co-stimulatory domain and/or a 41BB co-stimulatory domain and/or OX40 co-stimulatory domain. For example, the endodomain can comprise only one co-stimulatory domain and the co-stimulatory domain is a CD28 co-stimulatory domain. In another example, the endodomain can comprise only one co-stimulatory domain and the co-stimulatory domain is a 41BB co-stimulatory domain. In yet another example, the endodomain can comprise only one co-stimulatory domain and the co-stimulatory domain is an OX40 co-stimulatory domain.

A costimulatory domain from OX40 can, for example, have the sequence: ALYLLRRDQRLPPDAHKPPGGGSFRTPIQEEQADAHSTLAKI (SEQ ID NO: 5). A costimulatory domain from CD28 can, for example, have the sequence. RSKRSRLLHSDYMNMTPRRPGPTRKHYQPYAPPRDFAAYRS (SEQ ID NO: 6). A costimulatory domain from 41BB can, for example, have the sequence. KRGRKKLLYIFKQPFMRPVQTTQEEDGCSCRFPEEEEGGCEL (SEQ ID NO: 7). Preferably, the encoded costimulatory signaling domain comprises a functional signaling domain of a protein chosen from one or more of CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8ct, CD8fi, IL2Rp, IL2Ry, IL7Ra, ITGA4, VLA1, CD49a, ITGA4, IA4, CD49D, ITGA6, VLA-6, CD49f, ITGAD, CDIId, ITGAE, CD103, ITGAL, CD18, LFA-1, ITGAM, CD11b, ITGAX, CD1 lc, 1TGB I, CD29, ITGB2, CD18, LFA-1, ITGB7, TNFR2, TRANCE/RA KL, DNAM1 (CD226), SLAMF4 (CD 244. 2B4), CD84, CD96 (Tactile), CEACAMI, CRTAM, Ly9 (CD229), CD 160 (BY55), PSGL1, CD 100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, or NKG2D. In additional aspects, the co-stimulatory domain is a co-stimulatory domain of CD28, CD28T, OX40, 4-1BB/CD137, CD2, CD3 (alpha, beta, delta, epsilon, gamma, zeta), CD4, CDS, CD7, CD9, CD16, CD22, CD27, CD30, CD 33, CD37, CD40, CD 45, CD64, CD80, CD86, CD134, (1) 137, CD154, PD-1, ICOS, lymphocyte function-associated antigen-1 (LFA-1 (CD1 1a/CD 18), CD247, CD276 (B7-H3), LIGHT (tumor necrosis factor superfamily member 14; TNFSF14), NKG2C, Ig alpha (CD79a), DAP-10, Fc gamma receptor, MHC class I molecule, TNF, TNFr, integrin, signaling lymphocytic activation molecule, BTLA, Toll ligand receptor, ICAM-1, B7-H3, CDS, ICAM-1, GITR, BAFFR, LIGHT, HVEM (LIGHTR), KIRDS2, SLAMF7, p80 (KLRFl), NKp44, NKp30, NKp46, CD 19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CDlld, ITGAE, CD 103, ITGAL, CDlla, LFA-1, ITGAM, CDllb, ITGAX, CDlic, ITGB1, CD29, ITGB2, CD18, LFA-1, ITGB7, NKG2D, TNFR2, TRANCE RANKL, DNAM1 (CD226), SLAMF4 D244. 2B4), CD84, CD96 (Tactile), CEACAMI, CRT AM, Ly9 (CD229), CD160 (BY55), PSGL1, CDIOO (SEMA4D), CD69, SLAMF6 (NTB-A, Ly108), SLAM (SLAMF1, CD 150, IPO-3), BLAME (SLAMF8), SELPLG (CD 162), LTBR, LAT, GADS, SLP-76, PAG/Cbp, CD 19a, CD83 ligand. One of skill in the art will be able to determine the corresponding transmembrane regions from these polypeptides. Furthermore, any of the costimulatory domain sequences may contain from 1 to 5 amino acid modifications, which may be selected as discussed herein. In certain embodiments, the signaling domain comprises CD28, OX40, 4IBB, or a combination thereof.

The extracellular domain comprising the antigen recognition domain can be linked to the intracellular signaling domain via an extracellular spacer (also referred to herein as an extracellular hinge domain) and/or a transmembrane domain. The extracellular antigen binding domain and the transmembrane domain can be linked by an extracellular hinge domain or an extracellular spacer sequence. Preferably, the extracellular spacer or extracellular hinge domain sequence comprises one or more of a hinge region and/or a portion of an immunoglobulin heavy chain constant region (which may comprise CH1, a linker region, CH2 and/or CH3 domains) or any combination thereof, of a human immunoglobulin, i.e., IgA, IgD, IgE, IgG, and IgM. In certain embodiments, extracellular spacer or hinge domain comprises all or a portion of the hinge region of human IgD. In certain embodiments, extracellular spacer or hinge comprises all or a portion of the hinge region of human IgG1. In certain embodiments, the extracellular spacer or hinge comprises all or a portion of the hinge region of human IgD and all or a portion of the hinge region of human IgGl. In certain embodiments, the extracellular spacer or hinge comprises all or a portion of the hinge region of human IgD and all or a portion of the CH2 and CH3 domains of the heavy-chain constant region of human IgG1. In certain embodiments, the extracellular spacer or hinge comprises all or a portion of the hinge region of human IgD, all or a portion of the hinge region of human IgG1 and all or a portion of the CH2 and CH3 domains of the heavy chain constant region of human IgG1. In certain embodiments, the extracellular spacer or hinge comprises all or a portion of the hinge region of human IgG1 and all or a portion of the CH2 and CH3 domains of the heavy chain constant region of human IgG1. In certain embodiments, extracellular spacer or hinge comprises all of the hinge region of human IgD, all or a portion of the hinge region of human IgG1 and the heavy chain constant region comprises all or a portion of the CH2 and CH3 domains of human IgG1. Preferably the hinge region amino acid sequence comprises the hinge region amino acid sequence from an immunoglobulin, such from IgD or IgG1, wherein the amino acid sequence comprises from 1 to 5 amino acid modifications, which may be selected as discussed herein. Preferably, the CH2 and CH3 domains of the heavy chain constant region comprises the CH2 and CH3 domain immunoglobulin heavy chain constant region amino add sequence from an immunoglobulin, such from IgGl, wherein the amino acid sequence comprises from 1 to 5 amino acid modifications, which may be selected as discussed herein. In other aspects, the extracellular spacer or the extracellular hinge domain comprises the hinge region of a protein selected from the group consisting of CD8a, CD28, CD137, or a combination thereof. In certain aspects, the extracellular spacer or the extracellular hinge domain comprises the hinge region of CD8a. In any of the foregoing, the extracellular spacer may further comprise a linker, such as a linker having the sequence of Ser-Gly-Gly-Gly (SEQ ID NO: 8) or Ser-Gly-Gly-Gly-Gly (SEQ ID NO: 9), which may be present having from 1 to 10 copies, linking the extracellular spacer to the extracellular antigen binding domain.

In certain embodiments, the antigen recognition domain is linked to the transmembrane domain via a flexible linker. The flexible linker can be present in addition to the extracellular spacer or instead of the extracellular spacer described herein. In certain embodiments, the extracellular domain/antigen recognition domain is linked to the extracellular spacer via a flexible linker. The flexible linker can comprise, for example, glycine and serine. In certain aspects, the flexible linker is comprised of a polypeptide having the sequence of SEQ ID NO: 10 (Ser-Gly-Gly-Gly)ₙ or SEQ ID NO: 8 (Ser-Gly-Gly-Gly-Gly) wherein n is an integer from 1 to 10. The flexible linker can be a polypeptide comprising from about 1-25 amino acids, preferably about 1-15 amino acids, preferably about 1-10 amino acids, preferably about 4-24 amino acids, preferably about 5-20 amino acids, preferably about 5-15 amino acids and preferably about 5-12 amino acids. In certain aspects, the linker is (Ser-Gly-Gly-Gly)ₙ wherein n is 3.

The CAR of the invention can comprise a transmembrane domain that corresponds to, or is derived or obtained from, the transmembrane domain of any molecule known in the art. For example, the transmembrane domain can correspond to that of a CD8 molecule or a CD28 molecule. CD8 is a transmembrane glycoprotein that serves as a co-receptor for the T-cell receptor (TCR) and is expressed primarily on the surface of cytotoxic T-cells. The most common form of CD8 exists as a dimer composed of a CD8 and CD8β chain. CD28 is expressed on T-cells and provides co-stimulatory signals required for T-cell activation. A transmembrane domain from a CD8 polypeptide may have the sequence IYIWAPLAGTCGVLLLSLVITLYC (SEQ ID NO: 11), particularly amino acids 1-21, 1-23 or 1-24 of SEQ ID NO: 13). CD28 is the receptor for CD80 (B7.1) and CD86 (B7.2). A transmembrane domain from a CD28 polypeptide may have the sequence FWVLVVVG GVLACYSLLVTVAFIIFWV (SEQ ID NO: 12). Preferably, the CD8 and CD28 are human. Exemplary transmembrane domains of the CARs of the invention include, but are not limited to, all or a portion of a transmembrane domain from a polypeptide selected from: an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CDIIa, CD18), ICOS (CD278), 4-IBB (CD137), GITR, CD40, BAFFR, HVEM (LIGHTR), SLAMF7, NKp80 (KLRFl), CD160, CDI9, IL2R□, 1L2Ry, IL7R□, ITGA1, VLA1, CD49a, ITGA4, IA4, CD49D, ITGA6, VLA-6, CD49f, ITGAD, CDIId, ITGAE, CD 103, ITGAL, CD1 la, LFA-1, ITGAM, CDllb, ITGAX, CD1 lc, ITGB1, CD29, ITGB2, CD18, LFA-1, ITGB7, TNFR2, DNAM1 (CD226), SLAMF4 (CD244, 2B4), CD84, CD96 (Tactile), CEACAMI, CRTAM, Ly9 (CD229), CD 160 (BY55), PSGLI, CD100 (SEMA4D), SLAMF6 (NTB-A, Ly108), SLAM (SLAMF1, CD 150, IPO-3), BLAME (SLAMF8), SELPLG (CD 162), LTBR, PAG/Cbp, NKp44, NKp30, NKp46, NKG2D, and/or NKG2C. One of skill in the art will be able to determine the corresponding transmembrane regions from these polypeptides.

The CAR can comprise any one of the aforementioned transmembrane domains and any of the aforementioned hinge domain and any one or more of the aforementioned co-stimulatory domains. For example, the non-signaling CAR can comprise a CD28 transmembrane domain and a CD28 co-stimulatory domain. Furthermore, any of the transmembrane domain sequences may contain from 1 to 5 amino acid modifications, which may be selected as discussed herein.

The CAR can further comprise one or more of the following: an optional linker linking the antigen recognition domain to the hinge domain; a hinge domain comprising all or a portion of a hinge region of CD8a, CD28, or CD137; preferably, the hinge region of CD8a or CD28; a transmembrane region from CD28; and an optional costimulatory signaling domain as described herein; preferably, the CD28, OX40 and/or the 4-1BB co-stimulatory domain. In yet further aspects, the CAR can comprise an extracellular signal peptide. For example, the signal peptide can be the signal peptide of a protein selected from the group consisting of CD8a, CD28, GM-CSF, CD4, CD137, or a combination thereof.

As discussed above, the non-signaling CAR does not comprise or include a CD3 zeta (also referred to herein as CD3z or CD3 γδ signaling domain. Without wishing to bound by theory, the absence of a signaling domain in the non-signaling CAR γδ T cells may mitigate activation-induced cell death (AICD) which increases the survival or persistence of the CAR γδ T cells and thus prolong their effects. In addition, the absence of the CD3z signaling domain decreases the cytotoxicity of the non-signaling CAR γδ T cells to normal (non-cancerous) cells that express the target antigen to which the extracellular antigen binding domain can bind. The presence of the non-signaling CAR, however, enhances the affinity and/or avidity of the non-signaling CAR γδ T cells to malignant cells that express the target antigen, thus enhancing their cytotoxicity against target cells, for example, as compared with γδ T-cell that lack a CAR but are otherwise identical to the non-signaling CAR γδ T cells. For non-signaling CAR γδ T cells, no activation signal is transduced from the binding of the non-signaling CAR to the tumor antigen and this may mitigate on-target, off-tumor cytotoxicity. As discussed above, cytotoxicity is mediated by NKG2D signaling pathway of γδ T cells when they encounter specific tumor/stress antigens. In some embodiments, the cytotoxicity of the non-signaling CAR γδ T to normal cells is about 25% or less, about 20% or less, about 15% or less or about 10% or less (referring to the percentage of cells killed). In some embodiments, the cytotoxicity of the non-signaling CAR γδ T to normal cells is at least about 25%, at least about 20%, about 15%, or about at least about 10% less than the cytotoxicity of a comparable signaling CAR gd T cell. The cytotoxicity can be measured, for example, by flow cytometry.

The non-signaling CAR γδ T cells can display less activation-induced cell death (AICD) and tonic signaling issues as compared with comparable signaling CAR γδ T cells; this in turn, may promote the survival or persistence of non-signaling CAR γδ T cells. The invention thus encompasses methods of enhancing the persistence of CAR γδ T-cells in a subject undergoing treatment with a chemotherapeutic agent, the method comprising engineering the γδ T-cells to express the non-signaling CAR as described herein, wherein the non-signaling CAR γδ T-cells (or composition thereof) have enhanced persistence as compared to comparable signaling CAR γδ T-cells (or composition thereof), and further comprising administering the engineered γδ T-cells to the subject. In certain aspects, the invention is a method of enhancing the persistence of CAR γδ T-cells in a subject in need thereof, the method comprising engineering the γδ T-cells to express the non-signaling CAR as described herein, wherein the γδ T-cells co-express a cytokine selected from IL-15, IL-7 and/or IL-2, wherein the non-signaling CAR γδ T-cells (or composition thereof) have enhanced persistence as compared to comparable signaling CAR γδ T-cells (or composition thereof), and further comprising administering the engineered γδ T-cells to the subject non-signaling CAR γδ T-cells that co-express IL-15, IL-7 and/or IL-2. In additional aspects, because the non-signaling CAR γδ T-cells described herein are cytotoxic based on their interaction with stress antigens (and not the tumor antigen), the non-signaling CAR γδ T-cells show decreased killing of other CAR γδ T cells that present the tumor antigen on the T-cell cell surface via trogocytosis; for example, this decreased killing is as compared to that with comparable signaling CAR γδ-T cells, thus enhancing persistence.

The non-signaling CAR does not comprise or include an intracellular activation domain, and may or may not include a co-stimulatory domain. Without wishing to be bound by theory, the co-stimulatory domain (e.g., the CD28 co-stimulatory domain) can act as an intracellular anchor and/or to stabilize the construct within the cellular membrane and/or to enhance or prolong cell surface expression and/or to provide a co-stimulatory signal (signal 2).

In yet additional embodiments, the co-stimulatory domain does not provide a co-stimulatory signal or, in other words, signal 2. In a signaling CAR, the CD3z activation domain provides signal 1 and the co-stimulatory domain(s) provide signal 2; and both signals are generally believed to be required to activate the T-cell. The present invention encompasses embodiments wherein the co-stimulatory domain (such as only one co-stimulatory domain) is present but does not provide a co-stimulatory signal.

The invention additionally encompasses a method of enhancing the cytotoxicity of a γδ T-cells to tumor or cancer cells, for example, in a chemotherapeutic agent environment, the method comprising engineering the γδ T-cells to express a non-signaling CAR and optionally to express a survival factor and administering the engineered γδ T-cells to a patient in need thereof (for example a patient diagnosed with, suffering from, and/or being treated for cancer or tumor). The survival factor can for example, be a polypeptide that confers resistance to a chemotherapeutic agent as described herein. The non-signaling CAR γδ T-cell or a composition thereof has enhanced cytotoxicity to tumor cells, for example, in the chemotherapeutic agent environment (to which the survival polypeptide confers resistance), as compared to a γδ T-cell that lacks the non-signaling CAR (but is otherwise identical to the non-signaling CAR γδ T cell) or composition thereof. The invention additionally encompasses a method of enhancing the cytotoxicity of γδ T-cells to tumor or cancer cells (for example, leukemia or lymphoma cells), for example, in a chemotherapeutic agent environment, the method comprising engineering the γδ T-cells to express a non-signaling CD19 CAR and optionally to express a survival factor, (for example, a polypeptide that confers resistance to a chemotherapeutic agent as described herein) and administering the engineered γδ T-cells to a patient in need thereof. For example, the non-signaling CD19 CAR γδ T-cell or a composition thereof has enhanced cytotoxicity to cancer or tumor cells in the chemotherapeutic agent environment (to which the survival polypeptide confers resistance) than a γδ T-cell that lacks the non-signaling CAR (but is otherwise identical to the non-signaling CAR γδ T cell) or composition thereof. The invention further encompasses a method of enhancing the cytotoxicity of γδ T-cells to tumor or cancer cells (for example, leukemia or lymphoma cells), for example, in a chemotherapeutic agent environment, the method comprising engineering the γδ T-cells to express a non-signaling CD33 CAR and optionally to express a survival factor (for example, a polypeptide that confers resistance to a chemotherapeutic agent as described herein) and administering the engineered γδ T-cells to a patient in need thereof. For example, the non-signaling CD33 CAR γδ T-cell or a composition thereof has enhanced cytotoxicity to cancer or tumor cells in the chemotherapeutic agent environment (to which the survival polypeptide confers resistance) than a γδ T-cell that lacks the non-signaling CAR (but is otherwise identical to the non-signaling CAR γδ T cell) or composition thereof.

The invention additionally includes a method of treating a cancer or tumor in a patient in need thereof comprising administering an effective amount of the non-signaling CAR γδ T-cells as described herein, wherein the non-signaling CAR γδ T-cells have reduced cytotoxicity to non-cancerous cells as compared to comparable signaling CAR γδ T-cells. In certain aspects, the method comprises administration of an effective amount of the non-signaling CD19 CAR γδ T-cells as described herein (e.g., CD19 monoCAR γδ T cells and CD19 dualCAR γδ T cells, including tandem and separate dual CAR gd T cells), wherein the non-signaling CAR γδ T-cells have reduced cytotoxicity to non-cancerous B-cells as compared to comparable signaling CAR CD19 γδ T-cells. In another example, the method comprises administration of an effective amount of the non-signaling CD33 CAR γδ T-cells as described herein e.g., CD33 monoCAR gd T cells and CD33 dualCAR gd T cells, including tandem and separate dual CAR gd T cells), wherein the non-signaling CAR γδ T-cells have reduced cytotoxicity to non-cancerous myeloid cells as compared to comparable signaling CAR CD33 γδ T-cells.

The invention additionally includes a method of treating a cancer or tumor in a patient in need thereof comprising administering an effective amount of the non-signaling CAR γδ T-cells as described herein, wherein the non-signaling CAR γδ T-cells have reduced cytotoxicity to non-cancerous cells as compared to comparable signaling CAR γδ T-cells. In certain aspects, the method comprises administration of an effective amount of the non-signaling CD19 CAR γδ T-cells as described herein e.g., CD19 monoCAR gd T cells and CD19 dualCAR gd T cells, including tandem and separate dual CAR gd T cells), wherein the non-signaling CAR γδ T-cells have reduced cytotoxicity to non-cancerous B-cells as compared to comparable signaling CAR CD19 γδ T-cells. In another example, the method comprises administration of an effective amount of the non-signaling CD33 CAR γδ T-cells as described herein e.g., CD33 monoCAR gd T cells and CD33 dualCAR gd T cells, including tandem and separate dual CAR gd T cells), wherein the non-signaling CAR γδ T-cells have reduced cytotoxicity to non-cancerous myeloid cells as compared to comparable signaling CAR CD33 γδ T-cells.

The invention further includes a nucleic acid encoding the non-signaling CAR described herein or a vector comprising the nucleic acid. The nucleic acid encodes a non-signaling CAR comprising:
i. an extracellular antigen-binding domain that binds a tumor antigen;
ii. a transmembrane domain;
iii. an extracellular hinge domain; and
iv. optionally, a co-stimulatory domain;
   wherein the CAR does not comprise an intracellular T-cell activation domain. In certain aspects, the nucleic acid or vector further encodes a survival factor (such as a survival polypeptide) as described herein. In yet additional aspects, nucleic acid or vector encodes a polypeptide that confers resistance to a chemotherapeutic agent. In yet further aspects, the nucleic acid or vector further encodes a cytokine selected from IL-15, IL-2 and IL-7. In further aspects, the nucleic acid or vector encodes a self-cleaving peptide between the CAR and the survival peptide and/or the cytokine. Example of self-cleaving peptides include, for example, porcine teschovirus-1 2A (P2A) sequence, thosea asigna virus 2A (T2A), equine rhinitis A virus 2A (E2A), cytoplasmic polyhedrosis virus (BmCPV 2A), and flacherie virus (BmIFV 2A) of B. mori. In certain aspect, the nucleic acid or vector encodes a P2A sequences between the CAR and the survival polypeptide, such as MGMT or MDR1.

In yet additional aspects, the invention is directed to a vector (such as a lentiviral vector) comprising a nucleic acid that encodes a non-signaling CAR comprising:
i. an extracellular antigen-binding domain that binds a tumor antigen;
ii. a transmembrane domain;
iii. an extracellular hinge domain; and
iv. optionally, a co-stimulatory domain;
   wherein the CAR does not comprise an intracellular T-cell activation domain. In certain aspects, the vector further comprises a nucleic acid that encodes a survival factor (such as a survival polypeptide) as described herein. In yet additional aspects, the vector encodes a polypeptide that confers resistance to a chemotherapeutic agent. In yet further aspects, the vector encodes a cytokine selected from IL-15, IL-2 and IL-7. In further aspects, the vector encodes a self-cleaving peptide between the CAR and the survival peptide and/or the cytokine. Examples of self-cleaving peptides include, for example, porcine teschovirus-1 2A (P2A) sequence, thosea asigna virus 2A (T2A), equine rhinitis A virus 2A (E2A), cytoplasmic polyhedrosis virus (BmCPV 2A), and flacherie virus (BmIFV 2A) of B. mori. In certain aspects, the vector encodes a P2A sequences between the CAR and the survival polypeptide, such as MGMT or MDR1.

Specific non-signaling CARs include, for example, an antigen binding domain that binds a tumor antigen (for example, CD19 or CD33), a CD8a hinge domain, a CD28 transmembrane domain, a CD28 co-stimulatory domain, and MGMT. Another example of a non-signaling CAR can include, for example, an antigen binding domain that binds a tumor antigen (for example, CD19 or CD33), a CD8a hinge domain, a CD28 transmembrane domain, a CD28 co-stimulatory domain, and MDR1. A further example of a non-signaling CAR can include, for example, an antigen binding domain that binds a tumor antigen (for example, CD19 or CD33), a CD8a hinge domain, a CD28 transmembrane domain, a 4-1BB co-stimulatory domain, and MGMT. An additional example of a non-signaling CAR can include, for example, an antigen binding domain that binds a tumor antigen (for example, CD19 or CD33), a CD8a hinge domain, a CD28 transmembrane domain, a 4-1BB co-stimulatory domain, and MDR1. An extracellular c-myc or Flag peptide or other protein tag can be included for CAR-T detection and/or enrichment.

A non-signaling CAR according to the present invention can be produced by any means known in the art, though preferably it is produced using recombinant DNA techniques. A nucleic acid sequence encoding the several regions of the CAR can prepared and assembled into a complete coding sequence by standard techniques of molecular cloning (genomic library screening, PCR, primer-assisted ligation, site-directed mutagenesis, etc.). The resulting coding region is preferably inserted into an expression vector and used to transform a suitable expression host-cell line or primary cell, such as an immune effector cells, preferably a T lymphocyte cell line, and most preferably gamma delta T-cells (TS-T cells) and stem cells that differentiate into these cells, can also be used. In certain aspect, the primary cells are TS-T cells. As used herein, a “nucleic acid construct” or “nucleic acid sequence” is intended to mean a nucleic acid molecule, such as a DNA molecule, that can be transformed or introduced into an expression host-cell line, such as, but not limited to, a T-cell, and be expressed to produce a product (e.g., a chimeric receptor). Therefore, the invention further provides an isolated or purified nucleic acid sequence encoding the CARs of the invention. “Nucleic acid sequence” is intended to encompass a polymer of DNA or RNA, i.e., a polynucleotide, which can be single-stranded or double-stranded and which can contain non-natural or altered nucleotides. The terms “nucleic acid” and “polynucleotide” as used herein refer to a polymeric form of nucleotides of any length, either ribonucleotides (RNA) or deoxyribonucleotides (DNA). These terms refer to the primary structure of the molecule, and thus include double- and single-stranded DNA, and double- and single-stranded RNA. The terms include, as equivalents, analogs of either RNA or DNA made from nucleotide analogs and modified polynucleotides such as, though not limited to methylated and/or capped polynucleotides. In the nucleic acid construct employed in the present invention, the promoter is operably linked to the nucleic acid sequence encoding a CAR of the present invention, i.e., they are positioned so as to promote transcription of the messenger RNA from the DNA encoding the chimeric receptor. The promoter can be of genomic origin or synthetically generated. A variety of promoters for use in T-cells are well-known in the art. The promoter can be constitutive or inducible, where induction is associated with the specific cell type or a specific level of maturation, for example. Alternatively, a number of well-known viral promoters are also suitable. Promoters of interest include the 3-actin promoter, SV40 early and late promoters, immunoglobulin promoter, human cytomegalovirus promoter, retrovirus promoter, and the Friend spleen focus-forming virus promoter. The promoters may or may not be associated with enhancers, wherein the enhancers may be naturally associated with the particular promoter associated with a different promoter.

The various manipulations for preparing the non-signaling CARs of the invention can be carried out in vitro and the CAR chimeric construct can be introduced into vectors for cloning and expression in an appropriate cell using standard transformation or transfection methods. Thus, after each manipulation, the resulting construct from joining of the DNA sequences is cloned, the vector isolated, and the sequence screened to ensure that the sequence encodes the desired chimeric receptor. The sequence can be screened by restriction analysis, sequencing, or the like. Therefore, the invention comprises vectors encoding the CARs described herein or functional equivalents thereof.

As is well-known to one of skill in the art, various methods are readily available for isolating and expanding these cells from a subject. For example, using cell surface marker expression or using commercially available kits. It is contemplated that the chimeric construct can be introduced into the subject's own T-cells as naked DNA or in a suitable vector. Methods of stably transfecting T-cells by electroporation using naked DNA are known in the art. Naked DNA generally refers to the DNA encoding a chimeric receptor of the present invention contained in a plasmid expression vector in proper orientation for expression. Advantageously, the use of naked DNA reduces the time required to produce T-cells expressing the chimeric receptor of the present invention.

Therefore, the invention comprises cells (including primary cells) containing (i.e., transformed or transduced with) vectors encoding CAR(s) of the invention, as well as functional variants thereof. Preferably the cells are immune effector cells, preferably T-cells, a T lymphocyte cell line, and most preferably an autologous T lymphocyte cell line, a third party-derived T-cell line/clone, a transformed humoral or xenogenic immunologic effector cell line, for expression of the non-signaling CAR. Natural killer (NK) cells, macrophages, neutrophils, tumor-infiltrating-lymphocytes (TILs), lymphokine-activated killer (LAK) cells, memory T-cells, regulator}-T-cells, cytotoxic T lymphocytes (CTLs), gamma delta T-cells (γδ-T cells) and stem cells that differentiate into these cells, can also be used. Preferably γδ-T cells are used as the primary cells that are transformed or transduced as described herein. Once it is established that the transfected or transduced T-cell is capable of expressing the chimeric receptor as a surface membrane protein with the desired regulation and at a desired level, it can be determined whether the chimeric receptor is functional in the cell to provide for the desired signal induction. Subsequently, the transduced T-cells are reintroduced or administered to the subject to activate anti-tumor responses in the subject.

In one aspect, the invention encompasses cells (e.g., isolated cells), for example, γδ-T-cells, comprising (i.e., transformed or transduced with) a vector that encodes (i.e., directing the expression of) a non-signaling CAR of the present disclosure.

In an addition embodiment, the invention comprises cells (e.g., isolated cells), for example, γδ-T-cells, comprising (i.e., transformed or transduced with) a vector that encodes (i.e., directing the expression of) a non-signaling CAR of the present disclosure and a survival factor, such as a polypeptide that confers resistance to a chemotherapeutic agent as disclosed herein. Any CAR of the present disclosure may be used.

In yet another embodiment, the invention encompasses cells (e.g., isolated cells), for example, γδ-T-cells, comprising (i.e., transformed or transduced with) a vector that encodes (i.e., directing the expression of) a non-signaling CAR of the present disclosure and a cytokine selected from IL-15, IL-2 and/or IL-7. Any CAR of the present disclosure may be used.

In certain aspects, the transduction of the nucleic acid or the vector (e.g., the lentiviral vector) encoding a non-signaling CAR as described herein results in a transduction efficiency of at least about 10%, 20%, 30%, 40%, 45%, 50%, 55%, 60%, 65%, 70%, 75%, 80%, 85%, 90%, and 95%. In yet additional aspects, the vector copy number is between about 0.1 to about 10.

The γδ T-cells can naturally express a receptor for a stress-induced antigen (such as but not limited to, NKG2D); preferably, expression of the stress-induced antigen (to which the stress-induced antigen receptor binds) is increased by administration of the chemotherapeutic agent. For example, the γδ T-cells can naturally express a NKG2D receptor and as such can be utilized in a method of treatment comprising administration of a chemotherapeutic agent, wherein the administration of the chemotherapeutic agent increases the expression NKG2DL on tumor or cancer cells. In yet other aspects, the γδ-T-cells can comprise a vector (the same vector that encodes the CAR or a different vector) that encodes (i.e., directing the expression of) a stress-induced antigen receptor (such as but not limited to, NKG2D receptor).

As described above, the γδ-T-cells express a non-signaling CAR and can further express a survival factor and/or have been treated with a survival factor, wherein the survival factor is a DNA, RNA or polypeptide that confers resistance to a chemotherapeutic agent. In certain aspects, the cell expresses the survival factor and the survival factor is a polypeptide that confers resistance (to the γδ-T-cell) to the chemotherapeutic agent allows the γδ-T-cells to survive in a treatment environment created by the chemotherapeutic agent and/or allows the γδ-T-cells to survive in the tumor environment comprising the chemotherapeutic agent. In certain aspects, a single vector encodes the CAR and the polypeptide that confers resistance to a chemotherapeutic agent. In certain specific embodiments, a single vector encodes the CAR and a survival polypeptide selected from the group consisting of alkyl guanine transferase (AGT), P140K MGMT, O⁶ methylguanine DNA methyltransferase (MGMT), L22Y-DHFR, thymidylate synthase, dihydrofolate reductase, multidrug resistance protein 1 (MDR1), 5′ nucleotidase II, dihydrofolate reductase, and thymidylate synthase. In yet additional aspects, the single vector encodes the CAR and MGMT, or the CAR and MDR1. In certain embodiments, the cell comprising the vector that directs the expression of the CAR and the survival factor (and optionally a stress-induced antigen receptor) is an isolated or purified γδ T-cell. As discussed herein, the cells can be engineered to express a survival polypeptide that allows the cell, for example, the γδ-T-cell to survive in a treatment environment created a chemotherapeutic agent. Such cells which express a survival polypeptide are referred to herein as drug resistant (DR) cells and their use in therapy is referred to herein as “drug resistant immunotherapy” (DRI). DR cells and DRI is described in WO 2011/053750, the teachings of which are hereby incorporated by reference into the present application. As described herein, the survival polypeptide can be any polypeptide known in the art that provides resistance to a treatment regimen comprising a chemotherapeutic agent, and/or allows the cells comprising the survival polypeptide and the non-signaling CAR described herein to survive in a treatment environment created by the chemotherapeutic agent.

Exemplary chemotherapeutic agents are nucleoside-analog chemotherapy drug, alkylating agent, antimetabolite, antibiotic, topoisomerase inhibitor, mitotic inhibitor, differentiating agent, or hormone therapy agent and the survival factor provides resistance to the chemotherapeutic agent. In additional aspects, the chemotherapeutic agent is an alkylating agent. In certain embodiments, the survival polypeptide is MGMT, multidrug resistance protein 1 (MDRI), or 5′ nucleotidase II (NT5C2). In certain embodiments, the survival factor is an alkylguanine transferase (AGT; or alkylguanine-DNA-alkyltransferase) and the chemotherapeutic agent is an alkylating agent. In yet further aspects, the survival polypeptide is MGMT (including the P140K mutant of human O(6)-methylguanine-DNA-methyltransferase), and the chemotherapeutic agent is an alkylating agent such as carmustine (BCNU), lomustine (CCNU), and temozolomide. In certain aspects, the chemotherapeutic agent is temozolomide (TMZ). In certain aspects, the survival polypeptide is MDR1, MDR2, MDR3, MDR4, MDR5, MDR6, MDR7, MDR8, or MDR9. In additional aspects, the survival polypeptide is MDR1 and the chemotherapeutic agent is an anthracycline (e.g., daunorubicin), vinca alkaloids, epipodophyllotoxins, camptothecin, methotrexate (MTX), saquinavir, and mitoxantrone (MX) (Sodani et al. (2011). Multidrug resistance associated proteins in multidrug resistance. Chin J Cancer 31(2): 58-72). NT5C2 is a polypeptide known in the art to provide resistance to thiopurine chemotherapy (Tzoneva et al. (2013), Activating mutations in the NT5C2 nucleotidase gene drive chemotherapy resistance in relapsed ALL, Nat Med. 19(3): 368-371). Other survival polypeptide include, for example, a drug resistant variant of dihydrofolate reductase (L22Y-DHFR) and thymidylate synthase. In addition, numerous genes associated with resistance to platinum drugs (e.g., cisplatin, carboplatin and oxaliplatin) were described in Huang et al. (2021), A highly annotated database of genes associated with platinum resistance in cancer. Oncogene 40, 6395-6405; the contents of which are expressly incorporated by reference herein. In certain aspects, the survival polypeptide is MGMT or MDR1 However, other survival factors may be used depending on the chemotherapeutic agent being co-administered, the nature of the treatment environment (i.e., what other treatment regimens are being given to the patient in combination with the cells compositions of the present disclosure).

In additional aspects, the chemotherapeutic agent is an alkylating agent; a metabolic antagonist; a DNA demethylating agent; a substituted nucleotide; a substituted nucleoside; an antitumor antibiotic; an anthracycline; a plant-derived antitumor agent or a nitrosourea. Preferably the chemotherapeutic agent is selected from cisplatin; carboplatin; cyclophosphamide; cytarabine; etoposide; daunorubicin; fludarabine; idarubicin; methotrexate (MTX); trimethotrexate (TMTX); temozolomide; dacarbazine (DTIC), raltitrexed; S-(4-Nitrobenzyl)-6-thioinosine (NBMPR); 6-benzyguanidine (6-BG); a nitrosourea (rabinopyranosyl-N-methyl-N-nitrosourea (Aranose), Carmustine (BCNU, BiCNU), Chlorozotocin, Ethylnitrosourea (ENU), Fotemustine, Lomustine (CCNU), Nimustine, N-Nitroso-N-methylurea (NMU), Ranimustine (MCNU), Semustine, Streptozocin (Streptozotocin)); cytarabine; camptothecin; and a therapeutic derivative of any thereof. Preferably, the γδ T-cells have been genetically modified to encode alkyl guanine transferase (AGT), P140KMGMT, O⁶ methylguanine DNA methyltransferase (MGMT), L22Y-DHFR, thymidylate synthase, dihydrofolate reductase, or a multidrug resistance protein (such as MDR1).

In certain aspects, the γδ T-cells have been genetically modified to be resistant to at least two chemotherapeutic agents selected from: an alkylating agent; a metabolic antagonist; a DNA demethylating agent; a substituted nucleotide; a substituted nucleoside; an antitumor antibiotic; an anthracycline; a plant-derived antitumor agent and a nitrosourea. Preferably, the γδ T-cells have been genetically modified to be resistant to at least two chemotherapeutic agents selected from cisplatin; carboplatin; cyclophosphamide; cytarabine; etoposide; daunorubicin; fludarabine; idarubicin; methotrexate (MTX); trimethotrexate (TMTX); temozolomide; dacarbazine (DTIC), raltitrexed; S-(4-Nitrobenzyl)-6-thioinosine (NBMPR); 6-benzyguanidine (6-BG); a nitrosourea (rabinopyranosyl-N-methyl-N-nitrosourea (Aranose), Carmustine (BCNU, BiCNU), Chlorozotocin, Ethylnitrosourea (ENU), Fotemustine, Lomustine (CCNU), Nimustine, N-Nitroso-N-methylurea (NMU), Ranimustine (MCNU), Semustine, Streptozocin (Streptozotocin)); cytarabine; camptothecin; and a therapeutic derivative of any thereof. In certain aspects, at least one of the chemotherapeutic agents is TMZ, methotrexate, DTIC, BCNU, CCNU, MCNU, NMU or ENU, or a combination thereof. In yet additional aspects, at least one of the chemotherapeutic agents is cytarabine, an anthracycline (such as daunorubicin or idarubicin), cyclophosphamide, fludarabine, and rituximab.

A survival factor, including for example, the polypeptide that confers resistance to a chemotherapeutic agent (e.g., the chemotherapeutic agent being administered to the subject), can promote survival of the host cell expressing it in a treatment environment created by a chemotherapeutic agent, or survival in the presence of the chemotherapeutic agent when the cell survives in the presence of toxicity in the environment or the tumor microenvironment resulting from administration of the chemotherapeutic agent as part of the treatment. Chemotherapeutic agents for use with DRI (and γδ T-cells expressing the polypeptide that confers resistance to the chemotherapeutic agent) include, but are not limited to: alkylating agents (e.g., cyclophosphamide, ifosfamide, melphalan); metabolic antagonists (e.g., methotrexate (MTX), 5-fluorouracil or derivatives thereof); DNA demethylating agents (also known as antimetabolites; e.g., azacitidine): a substituted nucleotide; a substituted nucleoside; antitumor antibiotics (e.g., mitomycin, adriamycin); anthracyclines, plant-derived antitumor agents (e.g., vincristine, vindesine, TAXOL®, paclitaxel, abraxane); cisplatin; carboplatin; etoposide; and the like. Such agents may further include, but are not limited to, the anti-cancer agents trimethotrexate (TMTX); temozolomide (TMZ); raltitrexed; S-(4-Nitrobenzyl)-6-thioinosine (NBMPR); 6-benzyguanidine (6-BG); nitrosoureas (for example, bis-chloroethylnitrosourea, also known as BCNU and carmustine, lomustine, also known as CCNU, +/− procarbazine and vincristine (PCV regimen) and fotemustine); doxorubicin; daunorubicin, idarubicin, cyclophosphamide, cytarabine; camptothecin; and a therapeutic derivative of any thereof.

The engineered T6-T cell as described herein can further express a suicide gene. A “suicide gene” as used herein refers to a mechanism by which the non-signaling CAR-expressing cells described herein may be eradicated from a subject administered with the cells or a composition thereof, for example, in order to protect against a cascading inflammatory response or off-target cytotoxicity. The suicide gene system can, for example, be a Herpes Simplex Virus Thymidine Kinase (HSVTK)/Ganciclovir (GCV) suicide gene system, an inducible Caspase suicide gene system (Budde et al., PLoS One 2013 8(12):82742), codon-optimized CD20 (Marin et al., Hum. Gene Ther. Meth. 2012 23(6)376-86), CD34, a truncated EGFR (Wang X, Chang W-C, Wong C W, et al. A transgene-encoded cell surface polypeptide for selection, in vivo tracking, and ablation of engineered cells. Blood. 2011; 118(5):1255-1263. doi:10.1182/blood-2011-02-337360), a truncated CD19, or polypeptide RQR8 (Philip et al, and WO2013153391A, which is hereby incorporated herein by reference). An additional example of a suicide gene is the r-retrovirus SFG.iCaspase9.2A.DeltaCD19 which consists of iC9 linked, via a 2A-like sequence, to truncated human CD19 that serves as selectable marker. AP1903-inducible activation of the Caspase 9 suicide gene is achieved by expressing a chimeric protein (iC9), fused to a drug-binding domain derived from human FK506-binding protein (FKBP). The iC9 is quiescent inside cells until exposure to API 903, which cross-links the FKBP domains, initiates iCasp9 signaling, and induces apoptosis of the gene-modified cells. The gene and API 903 is available from Bellicum Pharmaceuticals (Houston, TX).

The γδ-T-cell expressing the non-signaling CAR can further express a reporter gene.

The γδ-T-cell expressing the non-signaling CAR can further expresses a receptor for a stress-induced antigen. In certain aspects, the γδ T-cell naturally expresses the receptor for a stress-induced antigen, for example, NKGD2 receptor. In other aspects, the γδ T-cell is engineered to express the stress-induced antigen receptor. In certain embodiments, the cell expressing a non-signaling CAR of the present disclosure further comprises a gene encoding for the stress-induced antigen receptor, such as NKGD2 receptor. In certain embodiments, the stress-induced antigen receptor, including, but not limited to, the NKGD2 receptor is induced to an increased level on the γδ T-cell.

DRI TS-T cells (γδ T cells that express a survival factor) that express the non-signaling CAR can be produced by incorporating a nucleic acid construct coding for and capable of expressing the non-signaling CAR described herein and optionally, can further express a DNA, RNA or polypeptide that confers resistance to a polypeptide, and optionally other elements (for example, a suicide gene and/or a receptor for a stress-induced antigen and/or a cytokine). In certain embodiments, a single nucleic acid construct codes for the CAR and the survival factor, such as a polypeptide that confers resistance to the chemotherapeutic agent, as well as the additional optional elements (for example, a suicide gene and/or a receptor for a stress-induced antigen and/or the cytokine). In certain embodiments, separate nucleic acid constructs code for each the non-signaling CAR and the survival factor, such as a polypeptide that confers resistance to a chemotherapeutic agent, and the optional other elements (for example, a suicide gene and/or a receptor for a stress-induced antigen and/or the cytokine). In certain embodiments, a single nucleic acid construct codes for the CAR and the survival factor, such as a polypeptide that confers resistance to a chemotherapeutic agent and one or more nucleic acid constructs codes for the additional optional elements (for example, a suicide gene and/or a receptor for a stress-induced antigen and or cytokine).

The cell, such as the γδ-T cells that is engineered to express the non-signaling CAR and the DNA, RNA or polypeptide that confers resistance to the chemotherapeutic agent and/or the cytokine, can be administered as part of a composition. The composition can comprise the engineered TS-T cells and additional immune system cells. For example, the composition may comprise γδ T-cells expressing the CAR as described herein, and can further comprise NK cells and/or αβ T-cells. In certain aspects, the composition comprises the engineered γδ T-cells expressing a CAR described herein and an additional immune system cell, wherein the γδ T-cells are present at greater than or equal to about 50%, 55%, 60%, 65%, 70%, 75%, 80%, 85%, 90%, or 95% of the total cell population, for example, as determined by flow cytometry. In yet further aspects, the γδ T-cells are present at greater than or equal to about 50%, 55%, 60%, 65%, 70%, 75%, 80%, 85%, 90%, or 95% of the total viable cell population, for example, as determined by flow cytometry. In certain embodiments, the composition comprises the engineered γδ T-cells and NK cells, wherein the γδ T-cells are present at greater than or equal to about 50%, 55%, 60%, 65%, 70%, 75%, 80%, 85%, 90%, or 95% of the total cell population or the total viable cell population and the NK cells are present at less than or equal to about 35%, 30%, 25%, or 20% (for example, as determined by flow cytometry). In certain embodiments, the composition comprises the engineered γδ T-cells and αβ T-cells, wherein the γδ T-cells are present at greater than or equal to 50%, 55%, 60%, 65%, 70%, 75%, 80%, 85%, 90%, or 95% of the total cell population or the total viable cell population, for example, as determined by flow cytometry. In additional aspects, the composition comprises the αβ T-cells at less than or equal to 5% of the total cell population or the total viable cell population, for example, as determined by flow cytometry. In certain embodiments, the composition comprises the engineered γδ T-cells, αβ T-cells and NK cells, wherein the γδ T-cells are present at greater than or equal to 50%, 55%, 60%, 65%, 70%, 75%, 80%, 85%, 90%, or 95% of the total cell population or the total viable cell population, for example as determined by flow cytometry. In certain embodiments, the αβ T-cells are present at less than or equal to 5% of the total cell population or the total viable cell population, and the NK cells are present at less than or equal to about 35%, 30%, 25%, or 20% of the total cell population or the total viable cell population, as determined by flow cytometry.

In certain aspects, therapeutic compositions for administration to a patient comprising optionally enriched and/or optionally expanded population of γδ T-cells comprise about 5×10⁸ 76 T-cells/kg or less of a patient's weight. In certain additional aspects, therapeutic compositions for administration to a patient comprising optionally enriched and/or optionally expanded population of δδ T-cells comprise about 5×10⁷ γδ T-cells/kg or less of a patient's weight. In further aspects, therapeutic compositions for administration to a patient comprising optionally enriched and/or optionally expanded population of γδ T-cells comprise about 5×10⁶ γδ T-cells/kg or less of a patient's weight.

Methods for isolating γδ T-cells either from a patient to be treated or from another source, as described, for example, by Lamb L. S. in U.S. Pat. No. 7,078,034, incorporated herein by reference in its entirety.

As described above, the use of the survival factor, including, for example, the polypeptide that confers resistance to a chemotherapeutic agent (such as the MGMT polypeptide), enables the compositions comprising the engineered γδ T-cells of the present disclosure (including a DRI γδ T-cells) to survive in a treatment environment created by the chemotherapeutic agent at a time when the tumor is stressed. The stress effect on the tumor (e.g., by the chemotherapeutic agent) in certain embodiments increases the expression of stress antigens, which are recognized by receptors, such as the NKG2D receptor, on the γδ T-cells. The dual effect of inducing stress antigens and decreasing regulatory T-cells with chemotherapy significantly improve tumor reduction over either individual regimen. Gene modification (expressing the survival polypeptide) and/or treatment with a survival factor as described herein protects the compositions of the present disclosure from the lymphodepleting effects of a chemotherapy regimen and allows the cell compositions of the present disclosure specific access to the tumor via TAA combined with unimpaired T-cell cytotoxic function at the time that malignant-cells are maximally stressed by chemotherapy. The use of DRI in combination with a non-signaling CAR in accordance with the invention is referred to herein as “DRI non-signaling CAR” therapy, is believed to significantly prolong survival and reduce tumor burden and time to recurrence when compared with either chemotherapy (for example, TMZ) treatment alone or TS T-cell infusion, for example, alone and do so without significant adverse systemic or neurologic consequences. In addition, as discussed above, the non-signaling CAR γδ T cells which lack the T-cell activating domain are not activated upon binding of the CAR to the antigen on the tumor or cancer cells. Instead, the γδ T cells bind stress ligands on the surface of tumor or cancer cells and the expression of these stress ligand can be increased by chemotherapy.

The compositions described herein can be delivered as a pharmaceutical composition, or made into an implant appropriate for administration in vivo, with appropriate carriers or diluents, which further can be pharmaceutically acceptable. The means of making such a composition or an implant have been described in the art. Where appropriate, the engineered γδ T-cells described herein can be formulated into a preparation in semisolid or liquid form, such as a capsule, solution, injection, inhalant, or aerosol, in the usual ways for their respective route of administration. Means known in the art can be utilized to prevent or minimize release and absorption of the composition until it reaches the target tissue or organ, or to ensure timed-release of the composition. Desirably, however, a pharmaceutically acceptable form is employed which does not ineffectuate the cells expressing the CAR. Thus, desirably the cells expressing the CAR as described herein can be made into a pharmaceutical composition containing a balanced salt solution, for example, Hanks' balanced salt solution, or normal saline. Therefore, the invention includes pharmaceutical compositions comprising γδ T-cells expressing a non-signaling CAR of the present disclosure, and specifically includes γδ T-cells expressing a non-signaling CAR and expressing the polypeptide that confers resistance to a polypeptide, and/or γδ T-cells expressing a non-signaling CAR and expressing the cytokine selected from the group consisting of IL15, IL2 and IL7, and/or γδ T-cells expressing a non-signaling CAR that comprises only one co-stimulatory domain, or a combination of any of thereof.

The pharmaceutical composition can be used alone or in combination with other well-established agents useful for treating cancer, for example, a chemotherapeutic agent as described herein. Whether delivered alone or in combination with other agents, the pharmaceutical composition of the present invention can be delivered via various routes and to various sites in a mammalian, particularly human, body to achieve a particular effect. One skilled in the art will recognize that, although more than one route can be used for administration, a particular route can provide a more immediate and more effective reaction than another route. For example, intradermal delivery may be advantageously used over inhalation for the treatment of melanoma. Local or systemic delivery can be accomplished by administration comprising application or instillation of the formulation into body cavities, inhalation or insufflation of an aerosol, or by parenteral introduction, comprising intramuscular, intravenous, intraportal, intrahepatic, peritoneal, subcutaneous, or intradermal administration.

The composition can be provided in unit dosage form wherein each dosage unit, e.g., an injection, contains a predetermined amount of the composition, alone or in appropriate combination with oilier active agents. The term unit dosage form as used herein refers to physically discrete units suitable as unitary dosages for human and animal subjects, each unit containing a predetermined quantity of the composition of the present invention, alone or in combination with other active agents, calculated in an amount sufficient to produce the desired effect, in association with a pharmaceutically acceptable diluent, carrier, or vehicle, where appropriate. The specifications for the novel unit dosage forms of the present invention depend on the particular pharmacodynamics associated with the pharmaceutical composition in the particular subject. Preferably, a therapeutically effective amount or sufficient number of the engineered γδ T-cells, administered alone or in combination with a therapeutic agent, is introduced into the subject such that a long-term, specific, response is established. In one embodiment, the response includes inhibition of cancer. In one embodiment, the response is the reduction in size of a tumor or elimination of tumor growth or regrowth or a reduction in metastasis to a greater degree than would otherwise result in the absence of the treatment with the engineered γδ T-cells or composition thereof. In certain aspects, the therapeutically effective amount results in at least about 5%, 10%, 20%, 30%, 40%, 50%, 60%, 70%, 80%, 90%, 95%, 98%, or 100% decrease in tumor size when compared that in the absence of the engineered CAR or in the absence of the γδ T-cells. Accordingly, the therapeutically effective amount takes into account the route of administration and the number of engineered cells should be such that a sufficient number of so as to achieve the desired therapeutic response. Furthermore, the amounts of the γδ T-cells of the present disclosure and/or additional cells included in the compositions described herein (e.g., the amount per each cell to be contacted or the amount per certain body weight) can vary in different applications. In certain non-limiting examples, the concentration of the cells can be sufficient to provide in the subject being treated at least from about 1×10⁵ to about 1×10¹⁰ cells, even more desirably, from about 1×10⁷ to about 5×10⁸ cells, although any suitable amount can be utilized either above, e.g., greater than 5×10⁸ cells, or below, e.g., less than 1×10⁷ cells. The dosing schedule can be based on well-established cell-based therapies or an alternate continuous infusion strategy can be employed.

These amounts provide general guidance to be utilized by the practitioner upon optimizing the method of the present invention for practice of the invention. The recitation herein of such ranges by no means precludes the use of a higher or lower amount of a component, as might be warranted in a particular application. For example, the actual dose and schedule can vary depending on whether the compositions are administered in combination with other pharmaceutical compositions, or depending on inter-individual differences in pharmacokinetics, drug disposition, and metabolism. One skilled in the art can readily make any necessary adjustments in accordance with the exigencies of the particular situation. Suitable doses for a therapeutic effect, for example, would be between about 10¹ and about 10¹⁰ cells per dose, preferably in a series of dosing cycles. In certain examples, the dosing regimen consists of four one-week dosing cycles of escalating doses, starting at about 1 (10⁵ cells on Day 0, increasing incrementally up to a target dose of about 10¹⁰ cells by Day 5. Suitable modes of administration include intravenous, subcutaneous, intracavitary (for example by reservoir-access device), intraperitoneal, and direct injection into a tumor mass.

The infused cells are able to kill cancer or tumor cells in the recipient. Unlike antibody therapies, cells expressing a CAR are able to replicate in vivo resulting in long-term persistence that can lead to sustained tumor control. The invention also includes a cellular therapy where 76-T cells are modified to transiently express a non-signaling CAR of the invention and the survival polypeptide, wherein the cells are infused to a recipient in need thereof. The infused cells are able to kill tumor cells or cancer cells in the recipient. Thus, in various aspects, the γδ-T cells administered to the patient, is present for less than one month, e.g., three weeks, two weeks, one week, after administration to the patient. In certain aspects, the cells administered to the patient, or their progeny, persist in the patient for at least four months, five months, six months, seven months, eight months, nine months, ten months, eleven months, twelve months, thirteen months, fourteen months, fifteen months, sixteen months, seventeen months, eighteen months, nineteen months, twenty months, twenty-one months, twenty-two months, twenty-three months, two years, three years, four years, or five years after administration of the cells to the patient. As described herein, the cancer to be treated can be a solid tumor or a hematological cancer. In some examples, the cancer to be treated can be of neuroectodermal origin. In certain aspects, the cancer is a malignant glioma, melanoma, neuroblastoma, medulloblastoma or small cell lung carcinoma. In yet additional aspects, the cancer is a hematologic or hematopoietic cancer.

In further aspects, γδ-T-cells can be a type of vaccine for ex vivo immunization and/or in vivo therapy in a mammal. In one aspect, the mammal is a human. With respect to ex vivo immunization, at least one of the following occurs in vitro prior to administering the cell or composition, including a pharmaceutical composition, comprising the cell into a mammal: i) expansion of the cells, ii) introducing one or more nucleic acids encoding the non-signaling CAR and optionally, the survival polypeptide to the cells and/or the cytokine selected from IL-15, IL-2 and IL-7 and/or iii) cryopreservation of the cells expressing or capable of expressing the CAR. Ex vivo procedures are well known in the art. Briefly, cells are isolated from a patient (e.g., a human) and genetically modified so as to express a CAR of the present disclosure (i.e., transduced or transfected in vitro with a vector expressing a CAR disclosed herein). The non-signaling CAR-modified cell can be administered to a patient to provide a therapeutic benefit. The patient is preferably a human and the non-signaling CAR-modified cell can be autologous with respect to the patient. Alternatively, the cells can be allogeneic, syngeneic or xenogeneic with respect to the patient.

The engineered γδ T-cells and one or more chemotherapeutic agent (e.g., the chemotherapeutic agent to which the survival factor confers resistance) can be co-administered. Such co-administration can encompass “simultaneous” or “concurrent delivery,” e.g., in the same or in separate compositions. In other aspects, co-administration encompasses separate administration but as part of the same treatment regimen. In certain aspect, the chemotherapeutic agent is administered before or concurrently with the engineered γδ T-cells. In additional aspects, the engineered γδ T-cells are co-administered with the one or more chemotherapeutic agent, wherein the chemotherapeutic agent causes increased expression of a stress ligand (e.g., NKG2DL) on the tumor or cancer cells; for example, the one or more chemotherapeutic agent is administered in an amount and in a manner/regiment resulting in increased express of the stress ligands. In certain aspects, the co-administration can be more effective than that of either treatment alone. The effect of the two treatments can be partially additive, wholly additive, or greater than additive. For example, co-administration can encompass administration of the γδ T-cells about 8 hours to about 72 hours after administration of one or more chemotherapeutic agent. In certain aspects, the engineered γδ T-cells are administered about 12 hours to about 36 hours after administration of the one or more chemotherapeutic agent; for example, the engineered γδ T-cells are administered about 24 hours after administration of the chemotherapeutic agent. In yet further aspects, co-administration encompasses administering the engineered γδ T-cells at the same time or at substantially the same time as the one or more chemotherapeutic agent. As used herein “substantially the same time” can encompass administration within the same treatment session.

The engineered γδ T-cells and the one or more chemotherapeutic agent can be administered during periods of active disorder, or during a period of remission or less active disease.

In further aspects, an additional therapeutic agent is administered in addition to the γδ T-cells and the chemotherapeutic agent. When administered in combination, the γδ T-cells and the chemotherapeutic agent and optionally, the additional therapeutic agent, the amount or dosage of one or all of the foregoing, can be administered in an amount or dose that is higher, lower or the same than the amount or dosage of each agent used individually, e.g., as a monotherapy. In certain embodiments, the amount or dosage of one or all of the foregoing, is lower (e.g., at least 20%, at least 30%, at least 40%, or at least 50%) than the amount or dosage of each agent used individually, e.g., as a monotherapy. In other embodiments, the amount or dosage of one or all of the foregoing, that results in a desired effect (e.g., inhibition of cancer) is lower (e.g., at least 20%, at least 30%, at least 40%, or at least 50% lower) than the amount or dosage of each agent used individually, e.g., as a monotherapy, required to achieve the same therapeutic effect.

The engineered γδ T-cells and the one or more chemotherapeutic agent can be administered in combination with an additional therapeutic treatment, such as, but not limited to, surgery, chemotherapy (e.g., an additional chemotherapeutic agent different from the chemotherapeutic agent to which the DR cells are resistant), checkpoint inhibitors, PARP inhibitors, radiation, immunosuppressive agents, such as cyclosporin, azathioprine, methotrexate, mycophenolate, and FK506, antibodies, or other immunoablative agents such as CAMPATH, anti-CD3 antibodies or other antibody therapies, Cytoxan (cyclophosphamide), fludarabine, FK506, rapamycin, mycophenolic acid, steroids, and cytokines. In yet additional aspects, the additional therapeutic agent is a checkpoint inhibitor, as described, for example, in WO2018/035413, the contents of which are expressly incorporated by reference herein. In further aspects, the additional therapeutic agent is a DDR inhibitor, including but not limited to PARP inhibitors as described, for example, in WO 2020/097306, the contents of which are expressly incorporated by reference herein.

The combination therapies disclosed herein can be administered to patient by various routes including, for example, orally or parenterally and can include but not be limited to, intravenously, intramuscularly, subcutaneously, intraorbitally, intracapsularly, intraperitoneally, intrarectally, intracisternally, intratumorally, intravasally, intradermally, intravaginally (e.g., vaginal suppositories), or topically (e.g., powders, ointments transdermal patch) or by passive or facilitated absorption through the skin using, for example, a skin patch or transdermal iontophoresis, respectively.

In some aspects, the total amount of an agent to be administered in practicing a method of the invention can be administered to a subject as a single dose, either as a bolus or by infusion over a relatively short period of time, or can be administered using a fractionated treatment protocol, in which multiple doses are administered over a prolonged period of time. One skilled in the art would know that the amount of the composition to treat a pathologic condition in a subject depends on many factors including the age and general health of the subject as well as the route of administration and the number of treatments to be administered. In view of these factors, the skilled artisan would adjust the particular dose as necessary.

The pharmaceutical compositions of the invention can be formulated to be compatible with the intended method or route of administration; exemplary routes of administration are set forth herein. Furthermore, the pharmaceutical compositions can be used in combination with other therapeutically active agents or compounds as described herein in order to treat or prevent the diseases, disorders and conditions as contemplated by the present disclosure.

The pharmaceutical compositions typically comprise a therapeutically effective amount of one or more agents and one or more pharmaceutically and physiologically acceptable formulation agents. Suitable pharmaceutically acceptable or physiologically acceptable diluents, carriers or excipients include, but are not limited to, antioxidants (e.g., ascorbic acid and sodium bisulfate), preservatives (e.g., benzyl alcohol, methyl parabens, ethyl or n-propyl, p-hydroxybenzoate), emulsifying agents, suspending agents, dispersing agents, solvents, fillers, bulking agents, detergents, buffers, vehicles, diluents, and/or adjuvants. For example, a suitable vehicle can be physiological saline solution or citrate buffered saline, possibly supplemented with other materials common in pharmaceutical compositions for parenteral administration. Neutral buffered saline or saline mixed with serum albumin are further exemplary vehicles. Those skilled in the art will readily recognize a variety of buffers that can be used in the pharmaceutical compositions and dosage forms contemplated herein. Typical buffers include, but are not limited to, pharmaceutically acceptable weak acids, weak bases, or mixtures thereof. As an example, the buffer components can be water soluble materials such as phosphoric acid, tartaric acids, lactic acid, succinic acid, citric acid, acetic acid, ascorbic acid, aspartic acid, glutamic acid, and salts thereof. Acceptable buffering agents include, for example, a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), and N-tris[Hydroxymethyl]methyl-3-aminopropanesulfonic acid (TAPS).

After a pharmaceutical composition has been formulated, it can be stored in sterile vials as a solution, suspension, gel, emulsion, solid, or dehydrated or lyophilized powder. Such formulations can be stored either in a ready-to-use form, a lyophilized form requiring reconstitution prior to use, a liquid form requiring dilution prior to use, or other acceptable form.

Preferably, the pharmaceutical composition is provided in a single-use container (e.g., a single-use vial, ampoule, syringe, or autoinjector (similar to, e.g., an EPIPEN®), whereas a multi-use container (e.g., a multi-use vial) is provided in other embodiments. Any drug delivery apparatus can be used to deliver IL-10, including implants (e.g., implantable pumps) and catheter systems, slow injection pumps and devices, all of which are well known to the skilled artisan. Depot injections, which are generally administered subcutaneously or intramuscularly, can also be utilized to release the polypeptides disclosed herein over a defined period of time. Depot injections are usually either solid- or oil-based and generally comprise at least one of the formulation components set forth herein. One of ordinary skill in the art is familiar with possible formulations and uses of depot injections.

The pharmaceutical compositions can be in the form of a sterile injectable aqueous or oleaginous suspension. This suspension can be formulated according to the known art using those suitable dispersing or wetting agents and suspending agents mentioned herein. The sterile injectable preparation can also be a sterile injectable solution or suspension in a non-toxic parenterally-acceptable diluent or solvent, for example, as a solution in 1,3-butane diol. Acceptable diluents, solvents and dispersion media that can be employed include water, Ringer's solution, isotonic sodium chloride solution, CREMOPHOR EL™ (BASF, Parsippany, N.J.) or phosphate buffered saline (PBS), ethanol, polyol (e.g., glycerol, propylene glycol, and liquid polyethylene glycol), and suitable mixtures thereof. In addition, sterile, fixed oils are conventionally employed as a solvent or suspending medium. For this purpose, any bland fixed oil can be employed, including synthetic mono- or diglycerides. Moreover, fatty acids such as oleic acid, find use in the preparation of injectables. Prolonged absorption of particular injectable formulations can be achieved by including an agent that delays absorption (e.g., aluminum monostearate or gelatin).

The pharmaceutical compositions can be in a form suitable for oral use, for example, as tablets, capsules, troches, lozenges, aqueous or oily suspensions, dispersible powders or granules, emulsions, hard or soft capsules, or syrups, solutions, microbeads or elixirs. Pharmaceutical compositions intended for oral use can be prepared according to any method known to the art for the manufacture of pharmaceutical compositions, and such compositions can contain one or more agents such as, for example, sweetening agents, flavoring agents, coloring agents and preserving agents in order to provide pharmaceutically elegant and palatable preparations. Tablets, capsules and the like contain the active ingredient in admixture with non-toxic pharmaceutically acceptable excipients which are suitable for the manufacture of tablets. These excipients can be, for example, diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc.

The tablets, capsules and the like suitable for oral administration can be uncoated or coated by known techniques to delay disintegration and absorption in the gastrointestinal tract and thereby provide a sustained action. For example, a time-delay material such as glyceryl monostearate or glyceryl distearate can be employed. They can also be coated by techniques known in the art to form osmotic therapeutic tablets for controlled release. Additional agents include biodegradable or biocompatible particles or a polymeric substance such as polyesters, polyamine acids, hydrogel, polyvinyl pyrrolidone, polyanhydrides, polyglycolic acid, ethylene-vinylacetate, methylcellulose, carboxymethylcellulose, protamine sulfate, or lactide/glycolide copolymers, polylactide/glycolide copolymers, or ethylenevinylacetate copolymers in order to control delivery of an administered composition. For example, the oral agent can be entrapped in microcapsules prepared by coacervation techniques or by interfacial polymerization, by the use of hydroxymethylcellulose or gelatin-microcapsules or poly (methylmethacrolate) microcapsules, respectively, or in a colloid drug delivery system. Colloidal dispersion systems include macromolecule complexes, nano-capsules, microspheres, microbeads, and lipid-based systems, including oil-in-water emulsions, micelles, mixed micelles, and liposomes. Methods for the preparation of the above-mentioned formulations will be apparent to those skilled in the art.

Formulations for oral use can also be presented as hard gelatin capsules wherein the active ingredient is mixed with an inert solid diluent, for example, calcium carbonate, calcium phosphate, kaolin or microcrystalline cellulose, or as soft gelatin capsules wherein the active ingredient is mixed with water or an oil medium, for example peanut oil, liquid paraffin, or olive oil.

Aqueous suspensions contain the active materials in admixture with excipients suitable for the manufacture thereof. Such excipients can be suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents, for example a naturally-occurring phosphatide (e.g., lecithin), or condensation products of an alkylene oxide with fatty acids (e.g., polyoxy-ethylene stearate), or condensation products of ethylene oxide with long chain aliphatic alcohols (e.g., for heptadecaethyleneoxycetanol), or condensation products of ethylene oxide with partial esters derived from fatty acids and a hexitol (e.g., polyoxyethylene sorbitol monooleate), or condensation products of ethylene oxide with partial esters derived from fatty acids and hexitol anhydrides (e.g., polyethylene sorbitan monooleate). The aqueous suspensions can also contain one or more preservatives.

Oily suspensions can be formulated by suspending the active ingredient in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents such as those set forth above, and flavoring agents can be added to provide a palatable oral preparation.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified herein.

The pharmaceutical compositions can also be in the form of oil-in-water emulsions. The oily phase can be a vegetable oil, for example olive oil or arachis oil, or a mineral oil, for example, liquid paraffin, or mixtures of these. Suitable emulsifying agents can be naturally occurring gums, for example, gum acacia or gum tragacanth; naturally occurring phosphatides, for example, soy bean, lecithin, and esters or partial esters derived from fatty acids; hexitol anhydrides, for example, sorbitan monooleate; and condensation products of partial esters with ethylene oxide, for example, polyoxyethylene sorbitan monooleate.

Formulations can also include carriers to protect the composition against rapid degradation or elimination from the body, such as a controlled release formulation, including implants, liposomes, hydrogels, prodrugs and microencapsulated delivery systems. For example, a time delay material such as glyceryl monostearate or glyceryl stearate alone, or in combination with a wax, can be employed.

Suppositories can be prepared by mixing the drug with a suitable non-irritating excipient which is solid at ordinary temperatures but liquid at the rectal temperature and will therefore melt in the rectum to release the drug. Such materials include, but are not limited to, cocoa butter and polyethylene glycols.

The pharmaceutical compositions suitable for use in accordance with the invention may be in any format (e.g., sprays for nasal or inhalation use) currently known or developed in the future.

The treatment methods described herein are particularly suitable for the treatment of cancer. Cancer cells can invade nearby tissues and can spread through the bloodstream and lymphatic system to other parts of the body. There are several main types of cancer, for example, carcinoma is cancer that begins in the skin or in tissues that line or cover internal organs. Sarcoma is cancer that begins in bone, cartilage, fat, muscle, blood vessels, or other connective or supportive tissue. Leukemia is cancer that starts in blood-forming tissue such as the bone marrow and causes large numbers of abnormal blood cells to be produced and enter the bloodstream. Lymphoma is cancer that begins in the cells of the immune system.

In certain aspects, the invention is directed to the treatment of a hematologic or hematopoietic cancer. For example, the non-signaling CD19 CAR γδ T cells described herein can be administered to a patient suffering from a hematologic or hematopoietic cancer. Such cancers include hematopoietic cancers (myelodysplastic cancer), myelodysplastic syndromes, pancreatic cancer, head and neck cancer, skin tumors, Minimal Residual Disease (MRD) among: acute Lymphocytic Leukemia (ALL), Acute Myelogenous Leukemia (AML), adult B-cell malignancies, including CLL (chronic lymphocytic leukemia), CML (chronic myelogenous leukemia), non-Hodgkin lymphoma (NHL), pediatric B-cell malignancies, including B lineage ALL (acute lymphocytic leukemia), multiple myeloma, lung cancer, breast cancer, ovarian cancer, prostate cancer, colon cancer, melanoma or other hematological and solid tumors, or any combination thereof.

In addition, the cancer can be a CD33-associated cancer and the non-signaling CD33 CAR γδ T cells described herein can be administered to a patient suffering therefrom. CD33-associated cancers include, but are not limited to, hematopoietic cancers, myelodysplastic syndrome, pancreatic cancer, head and neck cancer, skin tumors, Minimal Residual Disease (MRD) among: acute Lymphocytic Leukemia (ALL), Acute Myeloid Leukemia (AML), adult B-cell malignancies, including CLL (chronic lymphocytic leukemia), CML (chronic myeloid leukemia), non-Hodgkin's lymphoma (NHL), pediatric B-cell malignancies, including B lineage ALL (acute lymphocytic leukemia), multiple myeloma, lung, breast, ovarian, prostate, colon, melanoma, or other hematological and solid tumors, or any combination thereof.

The cancer can also be a CD19-associated cancer and the non-signaling CD19 CAR γδ T cells described herein can be administered to a patient suffering therefrom. Non-limiting examples of CD19-associated cancer include acute myeloid leukemia, myelodysplastic syndrome, chronic Myeloid Leukemia, Chronic Lymphocytic Leukemia, Non-Hodgkin Lymphoma, multiple myeloma, Plasmacytoma, Monoclonal gammopathy of undetermined significance, Waldenstrom's macroglobulinemia (lymphoplasmacytic lymphoma), Heavy chain disease, primary amyloidosis, Post-transplant lymphoproliferative disorder, Hodgkin lymphoma, MALT lymphoma, B cell Lymphoma, mantle cell lymphoma, (germinal center-like) diffuse large cell lymphoma, Burkitt's lymphoma, Bilineage leukemia, biphenotypic leukemia, Hairy cell leukemia, Precursor B acute lymphoblastic leukemia/lymphoma, Primary cutaneous follicle center lymphoma, follicular lymphoma, or Marginal Zone B-cell Non-Hodgkin's Lymphoma.

In yet additional aspects, the cancer is a B-cell lymphoma. Most non-Hodgkin lymphoma are B-cell lymphomas. Non-limiting examples of B-cell lymphoma include diffuse large B-cell lymphoma (DLBCL), follicular lymphoma, chronic lymphocytic leukemia (CLL), small lymphocytic leukemia (SLL), mantle cell lymphoma, marginal zone lymphoma, Burkitt lymphoma, Burkitt-like lymphoma, lymphoplasmacytic lymphoma (Waldenstrom macroglobulinemia), Hairy cell leukemia, primary central nervous system lymphoma, primary intraocular lymphoma.

In certain aspects, the cancer to tumor being treated can be an intracranial tumor. Intracranial tumors include, but are not limited to, gliomas, meningiomas, acoustic neuromas, pituitary adenomas, medulloblastomas, germ cell tumors and craniopharyngiomas. In some aspects, the cancer being treated in accordance with the invention is a CNS tumor including, but not limited to, intracranial and spinal ependymoma (excluding subependymoma); low grade infiltrative supratentorial astrocytoma/oligodendroglioma, medulloblastoma, anaplastic gliomas, glioblastoma, metastatic lesion of the CNS and primary CNS lymphoma.

In some aspects, the cancer being treated is a melanoma. In certain aspects, the cancer being treated is uveal melanoma.

In some aspects, the cancer being treated is a neuroendocrine or adrenal tumor. Examples include but are not limited to bronchopulmonary disease, GI tract, lung or thymus, pancreas, paraganglioma or pheochromocytoma.

In some aspects, the cancer being treated is non-Hodgkin's lymphoma including but not limited to mycosis fungoides and Sezary syndrome.

In some aspects, the cancer being treated is a soft tissue sarcoma. Examples include angiosarcoma, unresectable or progressive retroperitoneal/intra-abdominal soft tissue sarcoma, rhabdomyosarcoma, extremity/superficial trunk and/or head and neck cancer, or solitary fibrous tumor/hemangiopericytoma.

In some aspects, the cancer being treated is bone cancer. Examples include Ewing's sarcoma and mesenchymal chondrosarcoma.

In some aspects, the cancer being treated is uterine sarcoma, small cell lung cancer (SCLC) or Zollinger-Ellison syndrome.

In some aspects, the cancer being treated in accordance with the invention is a gynecologic cancer (e.g., cancers of the female reproductive system) including, but not limited to ovarian cancer, cancer of the fallopian tube(s), peritoneal cancer and breast cancer. In some aspects, the cancer being treated in accordance with the invention is ovarian cancer.

In some aspects, a cancer being treated in accordance with the invention is glioblastoma.

Brain tumors spread extensively within the brain but do not usually metastasize outside the brain. Gliomas are very invasive inside the brain, even crossing hemispheres. They do divide in an uncontrolled manner, though. Depending on their location, they can be just as life threatening as malignant lesions. An example of this would be a benign tumor in the brain, which can grow and occupy space within the skull, leading to increased pressure on the brain.

Also provided are kits comprising the pharmaceutical compositions typically comprise a therapeutically effective amount of one or more agents used in the combination therapies of the invention described herein. Kits typically include a label indicated as the intended use of the contents of the kits and instructions for use.

Any of the compositions or a combination of the compositions described herein can be comprised in a kit. In a non-limiting example, a chimeric receptor expression construct, one or more reagents to generate a chimeric receptor expression construct, cells for transfection of the expression construct, and/or one or more instruments to obtain autologous cells for transfection of the expression construct (such an instrument may be a syringe, pipette, forceps, and/or any such medically approved apparatus). The kits may comprise one or more suitably aliquoted compositions of the present invention or reagents to generate compositions of the invention. The components of the kits may be packaged either in aqueous media or in lyophilized form. The container means of the kits may include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the chimeric receptor construct and any other reagent containers in close confinement for commercial sale. Such containers may include injection or blow molded plastic containers into which the desired vials are retained, for example.

The kits are generally in the form of a physical structure housing various components, as described below, and can be utilized, for example, in practicing the methods described above. A kit can include a composition comprising one or more of the therapeutic agents used in the combination therapy of the invention (e.g. an engineered TS cells) provided in, e.g., one or more sterile containers, which can be in the form of a pharmaceutical composition suitable for administration to a subject. The pharmaceutical composition can be provided in a form that is ready for use or in a form requiring, for example, reconstitution or dilution prior to administration. When the compositions are in a form that needs to be reconstituted by a user, the kit can also include buffers, pharmaceutically acceptable excipients, and the like, packaged with or separately the therapeutic agent. When combination therapy is contemplated, the kit can contain the several agents separately or they can already be combined in the kit.

A kit of the invention can be designed for conditions necessary to properly maintain the components housed therein (e.g., refrigeration or freezing). A kit can contain a label or packaging insert including identifying information for the components therein and instructions for their use (e.g., dosing parameters, clinical pharmacology of the active ingredient(s), including mechanism(s) of action, pharmacokinetics and pharmacodynamics, adverse effects, contraindications, etc.).

Each component of the kit can be enclosed within an individual container, and all of the various containers can be within a single package. Labels or inserts can include manufacturer information such as lot numbers and expiration dates. The label or packaging insert can be, e.g., integrated into the physical structure housing the components, contained separately within the physical structure, or affixed to a component of the kit (e.g., an ampule, syringe or vial).

Labels or inserts can additionally include, or be incorporated into, a computer readable medium, such as a disk (e.g., hard disk, card, memory disk), optical disk such as CD- or DVD-ROM/RAM, DVD, MP3, magnetic tape, or an electrical storage media such as RAM and ROM or hybrids of these such as magnetic/optical storage media, FLASH media or memory-type cards. In some embodiments, the actual instructions are not present in the kit, but means for obtaining the instructions from a remote source, e.g., via an internet site, are provided.

### EXAMPLES

The following examples are offered by way of illustration and are not to be construed as limiting the invention in any way.

### Example 1: a Non-Signaling CD19 Gamma-Delta (γδ) T Cell-CAR to Preserve Healthy B Cells

γδ T cells are a unique subset of T lymphocytes that can directly kill malignant cells through the recognition of tumor and/or stress antigens that are not generally expressed on normal healthy tissue including hematopoietic cells. We hypothesized that we could harness the efficacy of CD19 CAR-T therapy and avoid on-target off-tumor cytotoxicity by incorporating a non-signaling CD19 CAR (ns19CAR) construct onto expanded and activated γδ T cells. We developed ns19CARs by removing the CD3z domain from the constructs. In addition, rIL-15 was co-expressed to enhance γδ T fitness and persistence.

The expression of the CAR was first validated by transduction of Jurkat T cells with either signaling CD19CAR or ns19CAR lentiviral vectors. Jurkat T-cells expressed the signaling CAR constructs (FIG. 3) and the non-signaling constructs (FIGS. 3 and 5). CAR-T activation was also measured by CD69 expression after co-culture of signaling CD19CAR transduced Jurkat T cells as well as after co-culture with non-signaling CD19CAR transduced Jurkat T cells. Jurkat cells transduced with a signaling CD19CAR demonstrated strong CD69 expression (FIG. 4: “CD19-Z”), while Jurkat cells transduced with ns19CAR showed no elevated expression when co-cultured with CD19+ Raji cells (FIG. 4: “CD19-noZ”). Next, the transduction of the ns19CAR in PBMC expanded V62+γδ T cells with zoledronate and IL-2 was tested. Non-signaling CD19 CAR lentiviral vector can efficiently transduce γδ T cells (FIG. 6) When transduced, nsl9-CAR γδ T cells were able to effectively kill CD19+ Nalm6 ALL cells at low effector-to-target ratios (E:T=0.25-4.0) and demonstrated enhanced (>1.5×) cytotoxicity compared to non-transduced control (NTC) (FIGS. 7 and 8). In comparison, only minimal cytotoxicity (10% or less) was observed when co-cultured with B cells from healthy donor PBMC, which are known to express CD19 (FIG. 7). Furthermore, there is no significant difference in cytotoxicity against CD19-lymphoblast K562 cells from the NTC or ns19CAR γδ T cells suggests that the enhanced cytotoxicity of ns19CAR γδ T cells is specific to CD19+ target cells (FIG. 7). Our results demonstrate that ns19CAR γδ cells may be able to selectively target malignant B cells while preserving healthy B cells.

In summary, non-signaling (ns) CAR γδ T-cells incorporate the natural function of γδ T-cells to discriminate between normal and distressed tissue. Additionally, nsCAR γδ T-cells fully preserve innate recognition and killing mechanisms of γδ T-cells and do not override innate functions with a separate CAR-directed signaling pathway. It has been shown that the nsCD19 CAR lentiviral vector construct described herein effectively transduces γδ T cells. Expanded and activated nsCD19 CAR transduced γδ T cells exhibit enhanced cytotoxicity against CD19+ Nalm6 ALL cell line as compared to non-transduced expanded and activated γδ T cells. Expanded and activated nsCD19 CAR transduced γδ T cells showed no difference in cytotoxic activity over unmodified expanded and activated γδ T-cells suggesting that enhanced cytotoxicity against Nalm-6 is specific to increased tropism of the CD19 bearing nsCD19 CAR T-cells to the tumor line. Non-signaling CD19 CAR γδ T-cells did not show significant killing at any E:T ratio against normal B cells from healthy donor PBMCs, avoiding on-target, off-tumor killing effects.

Non-signaling CD19 CAR γδ T cells have the potential to have similar effectiveness to standard CAR-T therapies but reduce off-tumor killing of healthy tissue that may express the tumor antigen target. Immunoglobulin replacement therapy is a costly and limited resource and associated with side effects without benefit (Hill et al. (2019), Blood Rev 38: 100596). Non-signaling CD19 CARs could reduce or eliminate the need for immunoglobulin replacement therapy, improve vaccine response and reduce infection risk. Non-signaling CD19 CAR γδ-T cells also have the potential to overcome ALL/Lymphoma resistance mechanisms via stress-antigen targeting even in cancers for which CD19 is down-regulated or lost. In addition, non-signaling CD19 CAR γδ T-cells could target AML-associated target antigens that currently are at unacceptable risk for on-target, off-tumor toxicity thereby improving depth of response and opening up approaches for transplant-ineligible patients. With respect to extracranial solid tumors, several potential tumor-associated antigens are widely expressed on normal tissues, some of which would compromise the use of direct-signaling CAR T-cells. Since nsCAR γδ T cells target the stress response on the malignant cell and use the CAR for localization instead of killing, these TAAs could be considered as druggable targets. Combinations of specific TAA-targeted design, Chemotherapy Resistant Cell Therapy (e.g., DRI), multiple dosing strategies, and other adjuvant approaches could provide long-lasting remission.

In addition, in contrast to the ap T cells, γδ T cells are not MHC-restricted and therefore are naturally capable of allogeneic cell therapy without significant risk of initiating graft-vs-host disease (GvHD). The unique combination of non-signaling CARs and γδ T cells shows potential as a next generation CAR-T therapy. The findings also provide insights to the development of future CAR therapies against other types of hematological and solid tumors.

To build the nsCD19-CAR constructs (FIGS. 2 and 5), gblocks double stranded DNAs encoding FMC63 scFv, CAR domains, and P2A-EGFP, Flag, IL-15, mCherry were synthesized by IDT DNA and cloned into transfer plasmid pDL171 by Gibson assembly cloning kit (New England Biolabs).

To package the 2nd generation lentiviral vector expression the signaling or non-signaling CD19CAR, the transfer, packaging and envelop plasmid DNAs were transfected into HEK-293T cells. Post-transfection cell supernatant was harvested and concentrated by PEG-8000 and NaCl solution. The titer of concentrated lentivirus was then quantitated by Lenti-X GoStix Plus (Takara Bio). The lentivirus was then aliquoted and stored at −80° C.

Jurkat T cells were transduced with CD19CAR or nsCD19CAR lentivirus and the transduction efficiencies were analyzed by flow cytometry gated on GFP+, Flag+ stained by mAB anti-flag (Abeam) or CD19CAR+ stained by mAB against FMC63-scfv (ACROBiosystems).

γδ T cells with higher than 50% γδ T were expanded from healthy donor apheresis product (Hemacare) and cultured in RPMI media (Cytiva HyClone) supplemented with 10% FBS (Cytiva HyClone), 25 mM HEPES (Thermo Scientific), 1×MEM NEAA (Cytiva HyClone), lx sodium pyruvate (Gibco) and 100 IU/mL human rIL-2. Expanded γδ T cells were transduced with the CAR construct expressing lentiviral vectors and maintained for at least 2 days before transduction efficiency analysis and cytotoxicity assays. Transduction efficiency of the non-signaling CAR γδ T cells were measured by flow cytometry.

Flow Cytometry Assays were conducted as follows. Activation: Lentivirus-transduced Jurkat T cells were co-cultured with Raji (CD19+) cells for 24 hours and stained with anti-CD69 antibody (BD). Cytotoxicity assay: Transduced γδ T cells were co-cultured with CFSE labeled Nalm6, PBMC-B or K562 cells at the indicated ratios for 16 or 48 hours then stained with 7-AAD(BD).

### Example 2: Validation of the s33CAR/Ns33CAR Constructs in Jurkat T Cells

As discussed above, it was hypothesized that a non-signaling γδ CAR platform (nsCAR) would target tumor associated antigens (TAA) also expressed by healthy cells but would discriminate between malignant and healthy cells using the innate recognition mechanisms characteristic of γδ T cells (see FIG. 10). This was tested in a proof-of-concept design by removing the CD33C domain from CAR-T constructs targeting the TAAs of CD19 (ns19CAR; see Example 1) or CD33 (ns33CAR). IL-15 was co-expressed to enhance γδ T-cell fitness and persistence. Both antigens are also broadly expressed on healthy lymphoid and myeloid cells. Discrimination between healthy and malignant target-expressing cells provides confidence that hematopoietic toxicities currently associated with traditional CAR-T therapy can be avoided, particularly with respect to CD33-expressing hematopoietic stem cells (HSC) and myeloid progenitors.

### Methods

i. CD19-CAR/CD33-CAR Lentiviral Vector Construction and Packaging

Synthesized gBlocks encoding a CD19 or CD33 signaling (CD3z+) CAR-T (s19CAR/s33CAR) and non-signaling (CD3z−) CAR-T (ns19CAR/ns33CAR) constructs were cloned, sequence verified and packaged into lentivirus (LV) in HEK293T cells (FIGS. 11A and 11B).

ii. Validation of CAR Constructs by Jurkat T Activation Co-Culture Assay

Jurkat T cells were transduced with CAR lentivirus and cultured with CD19+/CD33+ target cells for 24 hours and the activation of CD69 was analyzed by flow cytometry (FIGS. 12, 13A, 13B).

### iii. γδ T Mediated Cytotoxicity Assay

Activated and expanded Vδ2+γδ T cells from healthy donors were transduced with 15 ns19CAR/ns33CAR lentivirus and co-cultured with CFSE labeled target cells. For ns33CAR, LV transduced γδ T cells were cocultured with the CFSE labeled target cells HL-60 AML line (CD33+), K562 CML line (CD33+) and monocytes (CD33+) obtained from healthy donor for 24h (FIG. 15). γδ T cell mediated killing of target cell was assessed by flow cytometry (killed target cell %=CSFE+7AAD+/total CSFE+ cells) following co-culture. The cytotoxicity at E/T=X is normalized by the formula:

% cytotoxicity=(Killed target cell % at ET=X−Killed target cell % at E/T=0)/(1−Killed target cell % at E/T=0)

### RESULTS AND CONCLUSIONS

Expression of the CAR by transduction of Jurkat T cells with either signaling CD33 CAR (s33CAR) or non-signaling CD33 CAR (ns33CAR) lentiviral vectors was measured. Jurkat T-cells expressed the signaling CAR constructs and the non-signaling constructs (FIG. 12). CD69 activation was also measured after co-culture with KG-1 AML cells for negative control cells, s33CAR Jurkat T cells as well as ns33CAR Jurkat T cells (FIG. 13). Jurkat cells transduced with the signaling CAR demonstrated CD69 activation (“ssCAR”) while Jurkat cells transduced with ns33CAR showed no activation (“ns33 CAR”). The population of ssCAR+ and nsCAR+ populations after co-culture with KG-1 cells for 7 days was measured (e.g., ns33CAR transduced Jurkat cells were cocultured with KG-1 cells for 7 days). As shown in FIGS. 14A and 14B, the percent (%) ssCAR+ decreased with the length of co-culture. In contrast, there was no change in nsCAR+ population after extended co-culture with KG-1. In addition, as shown in FIG. 15, ns33CAR γδ T cells showed enhanced cytotoxicity (about 1.6×) against K562 cells at low E:T ratios. ns33CAR cells showed enhanced cytotoxicity (about 1.3×) against the highly resistant HL-60 AML cells line at higher E:T ratios. The ns33CAR toxicity against purified healthy monocytes is minimal and equivalent to non-transduced cells (UTD).

In conclusion, nsCAR-TS T cells show enhanced cytotoxicity against target tumor cells with minimal toxicity against healthy donor cells, potentially allowing CAR-T therapy against “undruggable” targets. In addition, the nsCAR platform may mitigate AICD in γδ T cells resulting from tonic signaling. Thus, the nsCAR platform for γδ T cells is a promising candidate for next generation CAR-T therapies and provides insights to CAR-T therapies that minimize on-target off-tumor toxicities. Promising strategies are currently under evaluation that will define and optimize for maximum cytotoxicity against highly resistant AML.

### Example 3: Gamma-Delta (γδ) CAR-T Cells Lacking the CD3K Signaling Domain Enhance Targeted Killing of AML Cells and Preserve Healthy Tissues

Chimeric antigen receptor T cell (CAR-T) therapy has shown remarkable efficacy against B cell malignancies, offering hope to patients with limited treatment options. However, extending this therapy to myeloid malignancies and solid tumors has proven challenging due to co-expression of targetable antigens on hematopoietic progenitors and healthy tissues. In this context, γδ T cells show promise, as they can directly identify and eliminate malignant cells via recognition of multiple tumor-associated stress antigens rarely expressed on normal tissues. We leveraged the tumor-sensing capabilities of γδ T cells with enhanced tumor localization by employing a non-signaling CAR (nsCAR) that excludes the CD3(domain, facilitating targeted tumor cell killing while sparing healthy tissues.

Second-generation lentiviral constructs for anti-CD33-scfv were designed for in vitro evaluation against acute myeloid leukemia (AML) lines HL-60, KG-1a, and MOLM-13 and the chronic myeloid leukemia (CML) line K-562. See FIG. 18 which shows the design of the construct. Additionally, CD33/CD123 dual-targeting nsCAR constructs (ns-dCAR) were tested to determine if the addition of CD123 targeting enhanced the therapeutic index. See FIG. 25 which shows the design of the construct. Whether a secreted IL-15 (sIL-15) would enhance CAR-T cell fitness was also tested.

Significant upregulation of CD69 was observed in Jurkat cells transduced with signaling CARs in co-culture with KG-la cells but not in those with nsCAR constructs (FIGS. 20A and 20B). A time-dependent reduction (43%) in the CD3(+CD33CAR+ population was also observed over a 7-day extended coculture while the nsCAR+ Jurkat population remained stable, suggesting potential mitigation of activation-induced cell death (AICD) (FIGS. 20A and 20B and FIGS. 22A to 22C). Expanded and activated γδ T cells from healthy donors were then transduced with nsCAR lentiviral vectors. The ns33CAR+γδ T cells exhibited enhanced killing capability against HL-60 (up to 1.3×) and K-562 (up to 1.6×) compared to unmodified γδ T cells (UTD) in a 24-hour cytotoxicity assay. Incorporation of sIL-15 into the nsCD33 CAR construct also increased killing across all 4 cell lines (up to 1.8× for HL-60, up to 2.6× for KG-1a, up to 2.0× for MOLM-13 and up to 2.0× for K-562) (FIGS. 24A-24D). Minimal cytotoxicity (<10%) was observed for nsCARs or UTDs against normal CD33+/CD123+ cells from healthy donor PBMC or CD34+ HPSCs (FIG. 24A). The ns-dCAR constructs did not improve in vitro AML killing compared to ns33CAR alone, although ns-dCARs generally exhibited lower transduction efficiency (FIGS. 33A to 33C).

In summary, these findings suggest that combining nsCAR constructs with 76 T cells may widen the therapeutic window to expand the reach of CAR-T therapy to cancers with limited target antigen expression on critical healthy tissues. Further optimization to improve integration and incorporation of membrane-bound IL-15 co-expression holds the potential to enhance both the efficacy and safety profiles of next-generation adoptive cell therapies against a broader spectrum of cancers.

The lentiviral constructs were constructed as described above. Jurkat T cells and gd T cells were transduced as described above and flow cytometric analysis and cytotoxicity was determined as described above. The anti-CD33 CAR construct included MY96 (Pfizer) which is a humanized mouse monoclonal anti-CD33. The 246 amino acid sequence is described in WO2016014576, the contents of which are expressly incorporated by reference herein. An exemplary CD33 CAR T cells is described in Kenderian et al. (2015), Leukemia 29: 1637-1647; the contents of which are expressly incorporated by reference herein.

While this invention has been particularly shown and described with references to preferred embodiments thereof, it will be understood by those skilled in the art that various changes in form and details may be made therein without departing from the scope of the invention encompassed by the appended claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference. The relevant teachings of all patents, published applications and references cited herein are incorporated by reference in their entirety.

1. An engineered γδ T-cell that expresses a chimeric antigen receptor (CAR), wherein the γδ T-cell further expresses a survival factor, wherein the survival factor is a DNA, RNA, or polypeptide that confers resistance to a chemotherapeutic agent, and further wherein:
the CAR comprises:
i. an extracellular antigen-binding domain that binds to a tumor antigen selected from CD19 and CD33;
ii. a transmembrane domain;
iii. an extracellular hinge domain; and
iv. an optional co-stimulatory domain;
wherein the CAR does not comprise an intracellular T-cell activation domain.

2. The γδ T-cell of claim 1, wherein the extracellular antigen-binding domain binds to CD19.

3. The γδ T-cell of claim 1, wherein the extracellular antigen-binding domain binds to CD33.

4. The γδ T-cell of claim 2, wherein the CAR is a monoCAR.

5. The γδ T-cell of claim 3, wherein the CAR is a monoCAR.

6. The γδ T-cell of claim 2, wherein the CAR is a tandem dualCAR.

7. The γδ T-cell of claim 3, wherein the CAR is a tandem dualCAR.

8. The γδ T-cell of claim 7, wherein the dual CAR binds the CD33 and CD123.

9. The γδ T-cell of claim 1, wherein the CAR comprises a co-stimulatory domain.

10. The γδ T-cell of claim 9 wherein the CAR comprises only one co-stimulatory domain.

11. The γδ T-cell of claim 1, wherein the γδ T-cell is engineered to express a cytokine selected from the group consisting of interleukin-15 (IL15), interleukin-2 (IL2), interleukin-7 (IL7), or a combination thereof.

12. The γδ T-cell of claim 10, wherein the co-stimulatory domain is selected from a CD28 co-stimulatory domain, an OX40 co-stimulatory domain, and a 4-1BB co-stimulatory domain, or a combination thereof.

13. (canceled)

14. (canceled)

15. The γδ T-cell of claim 1, wherein the extracellular antigen binding domain comprises:
i. an anti-CD19 antibody or an antigen binding fragment thereof, or
ii. an anti-CD33 antibody or antigen binding fragment thereof.

16. The γδ T-cell of claim 8, wherein the extracellular antigen-binding domain comprises:
i. an anti-CD33 antibody or antigen-binding fragment thereof, and
ii. an anti-CD123 antibody or an antigen binding fragment thereof, or an IL3 molecule.

17. The γδ T-cell of claim 15, wherein the extracellular antigen binding domain comprises an anti-CD19 scFv.

18. The γδ T-cell of claim 15, wherein the extracellular antigen binding domain comprises an anti-CD33 scFv.

19. The γδ T-cell of claim 16, wherein the extracellular antigen-binding domain comprises an anti-CD33 scFv and an IL3 molecule; optionally wherein the IL3 molecule is human IL3.

20. The γδ T-cell of claim 17, wherein the γδ T-cell further expresses IL15, and the CAR comprises only one costimulatory domain, and the only one costimulatory domain is a CD28 co-stimulatory domain.

21. The γδ T-cell of claim 18, wherein the γδ T-cell further expresses IL15, and the CAR comprises only one costimulatory domain, and the only one costimulatory domain is a CD28 co-stimulatory domain.

22. The γδ T-cell of claim 19, wherein the γδ T-cell further expresses IL15, and the CAR comprises only one costimulatory domain, and the only one costimulatory domain is a CD28 co-stimulatory domain.

23. The γδ T-cell of claim 1, wherein the survival factor is a polypeptide that confers resistance to a chemotherapeutic agent.

24. A pharmaceutical composition comprising the γδ T-cell of claim 1 and a pharmaceutically acceptable carrier.

25. A method of treating cancer or tumor in a subject in need thereof, the method comprising administering to said subject a composition comprising an effective amount of the engineered γδ T-cells of claim 1, the method further comprising co-administering to said subject the chemotherapeutic agent in an amount sufficient to increase stress antigen expression on the cancer or tumor cells.

26. (canceled)

27. (canceled)

28. (canceled)

29. (canceled)

30. An engineered γδ T-cell that expresses a chimeric antigen receptor (CAR), wherein the CAR comprises:
i. an extracellular antigen-binding domain that binds to CD19 or CD33;
ii. a transmembrane domain;
iii. an extracellular hinge domain; and
iv. an optional co-stimulatory domain;
wherein the CAR does not comprise an intracellular T-cell activation domain and wherein the γδ T cell is further engineered to express a cytokine selected from the group consisting of interleukin-15 (IL15), interleukin-2 (IL2), interleukin-7 (IL7), or a combination thereof.

31-57. (canceled)

58. An engineered γδ T-cell that expresses a chimeric antigen receptor (CAR), wherein the CAR comprises:
i. an extracellular antigen-binding domain that binds to CD19 or CD33;
ii. a transmembrane domain;
iii. an extracellular hinge domain; and
iv. only one co-stimulatory domain;
wherein the CAR does not comprise an intracellular T-cell activation domain.

59-88. (canceled)
